(19) United States
(12) Patent Application Publication
(10) Pub. No.: US 2023/0015009 A1
(43) Pub. Date: Jan. 19, 2023

(54) Methods of Decellularization and Recellularization of Organs and Portions of Organs

(71) Applicant: Miromatrix Medical Inc., Eden Prairie, MN (US)

(72) Inventors: Jeffrey ROSS, Chaska, MN (US); Aron STUMBRAS, Minneapolis, MN (US); Brett ANDERSON, Minneapolis, MN (US); Aleks KATANE, Chaska, MN (US); Emily BECK, Hopkins, MN (US); Joseph UZARSKI, Eden Prairie, MN (US)

(21) Appl. No.: 17/781,843

(22) PCT Filed: Dec. 4, 2019
(86) PCT No.: PCT/US2020/063460

§ 371 Date: Jun. 2, 2022

Related U.S. Application Data
(60) Provisional application No. 62/943,758, filed on Dec. 4, 2019.

(51) Int. Cl. C12M 3/00; A01N 1/02; C12M 1/00; C12M 1/34; C12N 5/071; A61L 27/36

(52) U.S. Cl. CPC C12M 21/08; A01N 1/0247; C12M 29/10; C12M 41/32; C12N 5/0697; A61L 27/3604; C12N 2533/90; C12N 2501/165; C12N 2501/155; C12N 2501/105; C12N 2501/12; C12N 2501/2302; A61L 2430/26

(57) ABSTRACT

Disclosed herein are compositions and methods to decellularize an isolated organ or portion thereof. Also disclosed herein are compositions and methods for treatment of disease utilizing a decellularized or recellularized organ. Also disclosed herein are methods of improving decellularization and/or recellularization of an isolated organ or portion thereof.

### CROSS-REFERENCE

This application claims the benefit of U.S. Provisional Application No. 62/943,758, filed Dec. 4, 2019, which application is incorporated herein by reference in its entirety.

### SUMMARY

Disclosed herein are compositions and methods for the decellularization and recellularization of organs and portions of organs. In an aspect, provided herein are compositions comprising an at least partially recellularized isolated organ or portion thereof comprising at least two different exogenous populations of cells engrafted thereon, wherein an at least partially recellularized isolated organ or portion thereof can clear ammonia at a rate of at least 0.1 mmol per hour from a fluid perfused through a vasculature as measured by a flow meter. In some cases, a fluid can comprise blood. In some cases, an at least partially recellularized isolated organ or portion thereof can be connected to a pump. In some cases, at least two exogenous populations of cells engrafted on an at least partially recellularized isolated organ or portion thereof can be allogeneic to an extracellular matrix of an at least partially recellularized isolated organ or portion thereof. In some cases, at least two exogenous populations of cells engrafted on an at least partially recellularized isolated organ or portion thereof can be autologous to an extracellular matrix of an at least partially recellularized isolated organ or portion thereof. In some cases, at least two exogenous populations of cells engrafted on an at least partially recellularized isolated organ or portion thereof can be xenogeneic to an extracellular matrix of an at least partially recellularized isolated organ or portion thereof. In some cases, an isolated organ or portion thereof can further comprise a perfusion solution. In some cases, a perfusion solution can comprise at least 120 pO₂ mmHg as measured by a Jenway® Model 970 dissolved oxygen meter and electrode. In some cases, a perfusion solution can comprise a growth factor, an immune modulating agent, a coagulation modulating agent, an antibiotic, a preservative, or any combination thereof. In some cases, a perfusion solution can comprise a growth factor, an immune modulating agent, a coagulation modulating agent, an antibiotic, a preservative, or any combination thereof. In some cases a growth factor can be selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 2 (BMP-2), Bone Morphogenic Protein 3 (BMP-3), Bone Morphogenic Protein 4 (BMP-4), Stromal Cell-Derived Factor 1 (SDF-1), Insulin like Growth Factor (IGF), Hepatocyte Growth Factor (HGF), and any combination thereof. In some cases, an immune modulating agent can comprise a cytokine, a glucocorticoid, an interleukin-2 receptor (IL2R) antagonist, a leukotriene antagonist, or any combination thereof. In some cases, a first population of exogenous cells can comprise embryonic stem cells, induced pluripotent stem cells (iPSCs), umbilical cord blood cells, hepatocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some cases, an at least partially recellularized isolated organ or portion thereof can comprise an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, or an at least partially recellularized pancreas or portion thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. In some cases, an at least partially recellularized isolated organ or portion thereof can be implanted in a subject.

Provided herein are compositions comprising an at least partially recellularized isolated organ or portion thereof comprising at least two exogenous populations of cells engrafted thereon, wherein the at least partially recellularized isolated organ or portion thereof comprises an at least partially intact vasculature that has a blood flow patency of at least 120 mL/min at about 15 mmHg as measured by a flow meter. In some cases, an at least partially recellularized isolated organ or portion thereof can be connected to a pump. In some cases, at least two exogenous populations of cells engrafted on an at least partially recellularized isolated organ or portion thereof can be allogeneic to an extracellular matrix of an at least partially recellularized isolated organ or portion thereof. In some cases, at least two exogenous populations of cells engrafted on an at least partially recellularized isolated organ or portion thereof can be autologous to an extracellular matrix of an at least partially recellularized isolated organ or portion thereof. In some cases, at least two exogenous populations of cells engrafted on an at least partially recellularized isolated organ or portion thereof can be xenogeneic to an extracellular matrix of an at least partially recellularized isolated organ or portion thereof. In some cases, an isolated organ or portion thereof can further comprise a perfusion solution. In some cases, a perfusion solution can comprise at least 120 pO₂ mmHg as measured by a Jenway® Model 970 dissolved oxygen meter and electrode. In some cases, a perfusion solution can comprise a growth factor, an immune modulating agent, a coagulation modulating agent, an antibiotic, a preservative, or any combination thereof. In some cases, a perfusion solution can comprise a growth factor, an immune modulating agent, a coagulation modulating agent, an antibiotic, a preservative, or any combination thereof. In some cases a growth factor can be selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 2 (BMP-2), Bone Morphogenic Protein 3 (BMP-3), Bone Morphogenic Protein 4 (BMP-4), Stromal Cell-Derived Factor 1 (SDF-1), Insulin like Growth Factor (IGF), Hepatocyte Growth Factor (HGF), and any combination thereof. In some cases, an immune modulating agent can comprise a cytokine, a glucocorticoid, an interleukin-2 receptor (IL2R) antagonist, a leukotriene antagonist, or any combination thereof. In some cases, a first population of exogenous cells can comprise embryonic stem cells, induced pluripotent stem cells (iPSCs), umbilical cord blood cells, hepatocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some cases, an at least partially recellularized isolated organ or portion thereof can comprise an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, or an at least partially recellularized pancreas or portion thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. In some cases, an at least partially recellularized isolated organ or portion thereof can be implanted in a subject.

Provided herein are compositions comprising an at least partially recellularized isolated organ or portion thereof comprising at least two exogenous populations of cells engrafted thereon, wherein the at least partially recellularized isolated organ or portion thereof comprises an at least partially intact vasculature comprising a circulating fluid, and wherein the at least partially recellularized isolated organ or portion thereof maintains an ammonia concentration of the circulating fluid at a level of less than about 0.4 mM in a time period of about 24 hours as measured by an ammonia analyzer. In some cases, a fluid can comprise blood. In some cases, an at least partially recellularized isolated organ or portion thereof can be connected to a pump. In some cases, at least two exogenous populations of cells engrafted on an at least partially recellularized isolated organ or portion thereof can be allogeneic to an extracellular matrix of an at least partially recellularized isolated organ or portion thereof. In some cases, at least two exogenous populations of cells engrafted on an at least partially recellularized isolated organ or portion thereof can be autologous to an extracellular matrix of an at least partially recellularized isolated organ or portion thereof. In some cases, at least two exogenous populations of cells engrafted on an at least partially recellularized isolated organ or portion thereof can be xenogeneic to an extracellular matrix of an at least partially recellularized isolated organ or portion thereof. In some cases, an isolated organ or portion thereof can further comprise a perfusion solution. In some cases, a perfusion solution can comprise at least 120 pO₂ mmHg as measured by a Jenway® Model 970 dissolved oxygen meter and electrode. In some cases, a perfusion solution can comprise a growth factor, an immune modulating agent, a coagulation modulating agent, an antibiotic, a preservative, or any combination thereof. In some cases, a perfusion solution can comprise a growth factor, an immune modulating agent, a coagulation modulating agent, an antibiotic, a preservative, or any combination thereof. In some cases a growth factor can be selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 2 (BMP-2), Bone Morphogenic Protein 3 (BMP-3), Bone Morphogenic Protein 4 (BMP-4), Stromal Cell-Derived Factor 1 (SDF-1), Insulin like Growth Factor (IGF), Hepatocyte Growth Factor (HGF), and any combination thereof. In some cases, an immune modulating agent can comprise a cytokine, a glucocorticoid, an interleukin-2 receptor (IL2R) antagonist, a leukotriene antagonist, or any combination thereof. In some cases, a first population of exogenous cells can comprise embryonic stem cells, induced pluripotent stem cells (iPSCs), umbilical cord blood cells, hepatocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some cases, an at least partially recellularized isolated organ or portion thereof can comprise an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, or an at least partially recellularized pancreas or portion thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. In some cases, an at least partially recellularized isolated organ or portion thereof can be implanted in a subject.

Provided herein is an at least partially recellularized isolated organ or portion thereof comprising a population of engrafted exogenous cells. In an aspect, a density of the population of the exogenous cells in a distal portion of the at least partially recellularized isolated organ or portion thereof comprises at most a 100% difference as compared to a density of the population of the exogenous cells in a proximal portion of the at least partially recellularized isolated organ or portion thereof, as measured by hematoxylin and eosin (H&E) staining of the population of the exogenous cells in the distal portion and the proximal portion of the at least partially recellularized isolated organ or portion thereof. In some cases, an isolated organ or portion thereof can further comprise a perfusion solution. In some cases, a perfusion solution can comprise at least 120 pO₂ mmHg as measured by a Jenway® Model 970 dissolved oxygen meter and electrode. In some cases, a perfusion solution can comprise a growth factor, an immune modulating agent, a coagulation modulating agent, an antibiotic, a preservative, or any combination thereof. In some cases a growth factor can be selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 2 (BMP-2), Bone Morphogenic Protein 3 (BMP-3), Bone Morphogenic Protein 4 (BMP-4), Stromal Cell-Derived Factor 1 (SDF-1), Insulin like Growth Factor (IGF), Hepatocyte Growth Factor (HGF), and any combination thereof. In some cases, an immune modulating agent can comprise a cytokine, a glucocorticoid, an interleukin-2 receptor (IL2R) antagonist, a leukotriene antagonist, or any combination thereof. In some cases, a first population of exogenous cells can comprise embryonic stem cells, induced pluripotent stem cells (iPSCs), umbilical cord blood cells, hepatocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some cases, an at least partially recellularized isolated organ or portion thereof can comprise an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, or an at least partially recellularized pancreas or portion thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. Also disclosed herein is a system comprising an at least partially recellularized isolated organ or portion thereof as disclosed herein operatively coupled to a pump. In some cases, a pump can comprise a peristaltic pump or a vacuum pump. In some cases, a system can further comprise a cannula, a perfusion apparatus, a holding container, a tubing, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof. In some cases, an at least partially recellularized isolated organ or portion thereof can be implanted in a subject.

Also disclosed herein is a method comprising introducing a second exogenous population of cells into an at least partially recellularized isolated organ or portion thereof comprising a first exogenous population of engrafted cells, wherein, prior to the introduction of the second population of exogenous cells, at least a portion of the first exogenous population of engrafted cells is functional as determined by: glucose consumption at a rate of at least about 10 mg/h; lactate production at a rate of at least about 30 mg/h; ammonia production at a rate of at least about 0.01 mmol/h; von Willebrand Factor production at a rate of at least about 0.1 ug/h; and any combination thereof. In some cases, an at least partially recellularized isolated organ or portion thereof can comprise an at least partially intact vasculature comprising a circulating fluid. In some cases, circulating fluid can comprise blood or a fraction thereof. In some cases, a circulating fluid can comprise: (i) a concentration of glucose of from about 0.5 g/L to about 4 g/L, (ii) a concentration of oxygen of from about 120 mmHg to about 400 mmHg, (iii) or any combination thereof. In some cases, at least 100% more blood perfusion rate can be observed as measured by an external blood loop, compared to an otherwise comparable isolated organ or portion thereof generated by engrafting the second exogenous population of cells onto a decellularized organ or portion thereof that lacks a functional subset of a first exogenous population of cells. In some cases, a first exogenous population of engrafted cells can comprise endothelial cells. In some cases, endothelial cells can comprise human vein endothelial cells (HUVECs). In some cases, a second exogenous population of cells comprises embryonic stem cells, induced pluripotent stem cells (iPSCs), umbilical cord blood cells, hepatocytes, cholangiocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), iPSC derived endothelial cells, differentiated stem cells or any combination thereof. In some cases, at least a portion of a second exogenous population of cells can comprise liver specific cells or kidney specific cells, wherein an exogenous population of cells can comprise liver specific cells and can comprise hepatocytes or cholangiocytes, and wherein an exogenous population of cells can comprise kidney specific cells and can comprise podocytes. In some cases, introducing cells can comprise using a cannula. In some cases, at least 25% more of any one of glucose consumption, lactate consumption, oxygen consumption, ribose consumption, and glycogen production can be observed in an at least partially recellularized isolated organ or portion thereof as compared to a comparable isolated organ or portion thereof generated by a comparable method absent a functional subset of a first exogenous population of engrafted cells before an introduction of a second exogenous population of cells. In some cases, a portion of a first exogenous population of engrafted cells can comprise at least 5% of a first exogenous population of engrafted cells. In some cases, an at least partially recellularized isolated organ or portion thereof can comprise an at least partially recellularized liver or portion thereof, and a second exogenous population of cells can be perfused into a liver or portion thereof via a hepatic vein. In some cases, an at least partially recellularized isolated organ or portion thereof can comprise an at least partially recellularized liver or portion thereof, and a second exogenous population of cells can be perfused into a liver or portion thereof via a bile duct. In some cases, a second population of cells can comprise hepatocytes. In some cases, at least one of a population of exogenous cells can be introduced by perfusing a recellularization solution into an at least partially recellularized isolated organ or portion thereof while an at least partially recellularized isolated organ or portion thereof can be at least partially submerged in a liquid that comprises a recellularization solution. In some cases, perfusing can be via a cannula. In some cases, perfusing can be antegrade. In some cases, perfusing can be retrograde.

Also disclosed herein is a method comprising: determining a concentration of a factor circulating in an at least partially recellularized isolated organ or portion thereof comprising a first population of cells engrafted thereon; and introducing into the at least partially recellularized isolated organ or portion thereof a second population of cells, wherein the first population of cells and the second population of cells are different, and wherein at least one of the first population of cells or the second population of cells are exogenous to the at least partially recellularized isolated organ or portion thereof. In some cases, a factor can comprise glucose, lactate, ammonia, oxygen, ribose, or glycogen. In some cases, a first population of cells can comprise endothelial cells. In some cases, a second population of cells can comprise embryonic stem cells, induced pluripotent stem cells (iPSCs), umbilical cord blood cells, hepatocytes, cholangiocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), iPSC derived endothelial cells, differentiated stem cells, or any combination thereof. In some cases, an at least partially recellularized isolated organ or portion thereof can comprise a liver, a kidney, a heart, a lung, a bowel, a skeletal muscle, a bone, a uterus, a bladder, a spleen, a brain, and a pancreas. In some cases, an at least partially recellularized isolated organ or portion thereof can be cultured in hyperoxic conditions following the introduction of the second population of cells. In some cases, hyperoxic conditions can comprise an oxygen concentration over 21%, 22%, 23%, or 24% pO₂ 140 mmHg as measured by a Jenway® Model 970 Dissolved Oxygen Meter and Electrode.

Also disclosed herein is a method comprising implanting into a subject an at least partially recellularized isolated organ or portion thereof as described herein. In some cases, a subject can have liver disease, hypertension, diabetes, heart failure, lung disease, or kidney disease. In some cases, liver disease can comprise cirrhosis, nonalcoholic steatohepatitis, hepatocellular carcinoma, metabolic disease, or any combination thereof. In some cases, a method can further comprise administering an immunosuppressive condition to a subject.

Provided herein is a method of at least partially treating kidney failure in a subject in need thereof, comprising grafting an at least partially recellularized kidney onto a circulatory system of the subject, wherein the at least partially recellularized kidney comprises at least a portion of an at least partially intact porcine kidney extracellular matrix comprising xenogeneic or allogeneic glomerular cells engrafted thereon prior to the grafting, wherein the grafting: (a) reduces a level of hematocrit in the blood of the subject, relative to a level of hematocrit in the blood prior to the grafting, (b) reduces an effluent protein concentration of the blood of the subject, relative to a protein concentration in the blood prior to the grafting; (c) is sufficient to produce an effluent flow rate that is comparable to a native porcine kidney; or (d) any combination thereof, thereby at least partially treating the kidney failure in the subject. In an embodiment, the glomerular cells comprise podocytes. In an embodiment, the glomerular cells comprise mesangial cells. In an embodiment, the glomerular cells comprise human cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference. U.S. Pat. Nos. 10,213,525 and 10,220,056 are incorporated by reference herein in their entireties.

### BRIEF DESCRIPTION OF THE DRAWINGS

The novel features of exemplary embodiments are set forth with particularity in the appended claims. A better understanding of the features and advantages will be obtained by reference to the following detailed description that sets forth illustrative embodiments, in which exemplary embodiments are utilized, and the accompanying drawings of which:

FIG. 1 depicts a schematic showing an initial preparation for a decellularization of a heart. An aorta, pulmonary artery, and superior vena cava are cannulated (A, B, C, respectively), and an inferior vena cava, brachiocephalic artery, left common carotid artery, and left subclavian artery are ligated. Arrows indicate the direction of perfusion in antegrade and retrograde.

FIG. 2 shows decellularization of an adult porcine heart over an about 48 hour time period. A 6-month-old porcine heart undergoing perfusion decellularization over a period of 48 hours. At least some native structure and at least some vasculature are preserved after decellularization. After 48 hours, a heart is at least partially decellularized.

FIG. 3 shows decellularization of an adult porcine liver over 24 hours. A 6-month-old porcine liver undergoing perfusion decellularization over a period of 24 hours. A native structure and vasculature remain preserved after decellularization.

FIG. 4 shows an increase in the flow rate after a radical generating compound (e.g., peracetic acid) is introduced into a perfusion decellularization process. An increase in flow rate occurs after a radical generating compound is added during perfusion decellularization of a porcine liver at a set pressure of 12 mmHg. A decellularization apparatus allows for a defined pressure to be specified and a flow rate may be adjusted in order to maintain a defined pressure.

FIG. 5 depicts improvements in flow rates of perfusion at constant pressure with the addition of a radical generating compound PAA during a decellularization process workflow.

FIG. 6A shows a porcine liver after decellularization having a particulate percent from 0-5%. FIG. 6B shows a porcine liver after decellularization having a particulate percent of 20%. FIG. 6C shows a porcine liver after decellularization having a particulate percent from 40-50%. FIG. 6D shows a porcine liver after decellularization having a particulate percent from 50-60%. FIG. 6E shows a porcine liver after decellularization having a particulate percent of 60%. FIG. 6F shows a porcine liver after decellularization having a particulate percent of 70%. FIG. 6G shows a porcine liver after decellularization having a particulate percent of 80%. FIG. 6H shows a porcine liver after decellularization having a particulate percent from 95-100%.

FIG. 7 shows an elution of particulate into a tubing connected to a portal vein cannula of a liver with visual particulate undergoing perfusion decellularization when perfusion is stopped for 10 minutes after about 24 hours of perfusion decellularization.

FIG. 8A shows decellularization of an adult porcine lung over 48 hours. A 6-month-old porcine lung undergoing perfusion decellularization over a period of 48 hours. A native structure and vasculature are preserved after decellularization. FIG. 8B depicts decellularization after 48 hours, a lung is completely decellularized.

FIG. 9A shows decellularization of an adult porcine kidney over 48 hours. A 6-month-old porcine kidney undergoing perfusion decellularization over a period of 48 hours. A native structure and vasculature are preserved after decellularization. FIG. 9B depicts decellularization of a kidney after 48 hours, a kidney is completely decellularized.

FIG. 10A shows an absorbance profile of a solution surrounding a liver with visible particulate (L281) after a 30 minute re-circulating water rinse. FIG. 10B shows an absorbance profile of effluent in a solution surrounding a liver with visible particulate L274 after 15 minutes of a re-circulating water wash.

FIG. 11 depicts processing of native and decellularized kidney scaffolds using DNA-binding florescence staining. Examination was conducted under immunofluorescent microscopy.

FIG. 12 shows a plot of the normalized peak load force values for samples that are stretched in increments of 4% strain and allowed to relax.

FIG. 13A depicts a representation of endothelial seeding used in the histological analysis. FIG. 13B depicts representative images of the bioreactor, histology after 21 days, and an exemplary vessel surface.

FIG. 14A depicts immunofluorescent staining, which demonstrates the distribution and engraftment of native kidney cells throughout the kidney in the proper location. FIG. 14B shows a bioreactor system enabling site-specific recellularization of the vasculature, and glomerular matrix, as shown in 3 distinct BEKs. FIG. 14C shows functional testing of BEKs during an in vivo blood loop (as described herein) shows a dramatic increase in ureter effluent flow rate in rBEKs compared to native or allograft pig kidneys due to decreased resistance. Introduction of glomerular cells (rBEK+Glom.) increases this resistance to limit fluid loss, while still enabling effluent generation. FIG. 14D shows the effluent generation as shown in FIG. 14C.

FIG. 15 depicts hepatocyte glucose consumption over time in normoxic vs hyperoxic conditions. Increased oxygen levels in the media results in a decreased glucose consumption and maintenance of native metabolic activity with lower levels of glucogenesis.

FIG. 16 depicts hepatocyte lactate production over time in normoxic vs hyperoxic conditions. Increased oxygen levels in the media results in decreased lactate production.

FIG. 17 depicts hepatocyte ammonia production over time in normoxic vs hypoxic conditions. Increased oxygen levels in the media results in decreased ammonia concentration.

FIG. 18 depicts hepatocyte ammonia clearance over time in normoxic vs. hyperoxic conditions. Increased oxygen levels results in the ability for hepatocytes to retain their ammonia clearing function.

FIG. 19 depicts media PO₂ levels in hepatocyte seeded systems incubated at various oxygen concentrations. Oxygen levels in the media sampled from the outflow of a hepatocyte seeded graft cultured in baseline normoxic conditions (21% O₂) were not hypoxic. The figure demonstrates that the enhancing effects of hyperoxic levels are in contrast to normoxic levels and not hypoxic levels.

FIG. 20 depicts glucose consumption of endothelial-only seeded grafts over time in normoxic vs hyperoxic conditions. Increased oxygen levels in the media of endothelial only seeded liver grafts results in no significant changes in glucose consumption.

FIG. 21 depicts representative flow rates following serial hepatocyte infusions in cell-free (hepatocytes only) and previously endothelialized porcine liver grafts (hepatocytes+HUVECs). Cell-free grafts experienced declining flow rates at 12 mmHg after serial hepatocyte infusions through the hepatic vein (500 million cells per infusion) when compared to reendothelialized grafts.

FIG. 22 depicts representative flow rates and corresponding pressures during perfusion bioreactor culture. Grafts seeded exclusively with hepatocytes require higher perfusion pressures to maintain flow rates of 250 mL/min than grafts previously seeded with HUVECs.

FIG. 23A and FIG. 23B depict blood perfusion patency and hepatocyte function in co-culture liver grafts. Grafts seeded with hepatocytes following reendothelialization with HUVECs remain competent for heparinized blood perfusion (FIG. 23A) and albumin production (FIG. 23B). 02EEH and 04EEH grafts were subjected to ex vivo heparinized (low ACT levels) blood perfusion on days 2 and 3 post hepatocyte seeding, respectively.

FIG. 24 depicts the characterization of bioengineered liver (BEL) constructs seeded with primary endothelial cells and hepatocytes. FIG. 24A depicts a schematic of the liver decellularization process. FIG. 24B shows a liver before decellularization. FIG. 24C shows a liver after decellularization. FIG. 24D and FIG. 24E show histological staining showing complete removal of cellular material from the decellularized scaffold. FIG. 24F, FIG. 24G, FIG. 24H, and FIG. 24I show retention of the extracellular matrix (ECM) proteins collagen I and collagen IV. FIG. 24J depicts decellularized livers mounted in custom bioreactors. FIG. 24K depicts the livers being perfused with antibiotic-free endothelial culture media for 72 h to confirm the sterility of the scaffold. FIG. 24L provides another exemplary bioreactor depicting organ liver perfusion.

FIG. 25 depicts representative flow cytometry analysis demonstrating enrichment of primary porcine hepatocytes.

FIG. 26 depicts Histological and functional characterization of bioengineered liver (BEL) constructs. FIG. 26A depicts H&E staining of representative Co-culture bioengineered liver (BEL) tissue sections fixed 48 h after seeding hepatocytes. FIG. 26B depicts CD31 & albumin immunofluorescent staining of cell lineage markers 48 h after seeding hepatocytes. FIG. 26C depicts CD31 & FAH immunofluorescent staining of cell lineage markers 48 h after seeding hepatocytes. FIG. 26D depicts CD31 & LYVE1 immunofluorescent staining of cell lineage markers 48 h after seeding hepatocytes. FIG. 26E depicts Albumin and LYVE1 immunofluorescent staining of cell lineage markers 48 h after seeding hepatocytes. FIG. 26F depicts vWF production in co-culture grafts before and after hepatocyte seeding. FIG. 26G depicts 24-hour average albumin production in co-culture grafts 24 and 48 h following hepatocyte seeding. FIG. 26H depicts ammonia clearance and FIG. 26I depicts urea production kinetics over 24 h following the addition of 1.6 mmol ammonium chloride to the bioreactor media. Error bars indicate standard deviation between independent replicates. FIG. 26J shows the presence of ammonia as a function of time in the BEL. FIG. 26K shows the presence of urea as a function of time in the BEL.

FIG. 27 depicts acute blood perfusion studies to assess vascular patency in BELs. FIG. 27A depicts a schematic of an in vitro blood perfusion circuit. 37° C. porcine blood is perfused at 12 mmHg through the PV with a peristaltic pump and returned to a reservoir through the IVC. FIG. 27B depicts summary plots of pressures and flow rates measured over 60 minutes during in vitro blood perfusion studies using HUVEC-only, Hepatocyte-only, or Co-culture BELs. Freshly explanted (Native) porcine livers and decellularized scaffolds (Decell) were included as benchmarks for idealized perfusion and rapid thrombosis, respectively. FIG. 27C depicts violin plots summarizing bioengineered liver (BEL) flow rates at 30 minutes from FIG. 27B. FIG. 27D depicts a schematic of an ex vivo blood perfusion model. A synthetic perfusion circuit was created by cannulation of the PV and IVC within a sedated pig. Blood flow was directed from the animal's cannulated PV to the bioengineered liver (BEL) PV and returned from the bioengineered liver (BEL) IVC into the animal's cannulated IVC. FIG. 27E depicts real-time angiography time lapse imaging following contrast infusion. Imaging was performed after 30 minutes of continuous blood perfusion.

FIG. 28 depicts a heterotopic implantation of co-culture BELs in a large animal model. FIG. 28A depicts a schematic of heterotopic bioengineered liver (BEL) implant surgical model. FIG. 28B depicts post-operative 3D-reconstruction from CT imaging demonstrating bioengineered liver (BEL) perfusion and devascularization of native liver. bioengineered liver (BEL) is outlined in yellow. FIG. 28C depicts representative transverse CT imaging of recipient animal at post-op, 24 h, and 48 h time points. bioengineered liver (BEL) is outlined in yellow. FIG. 28D depicts post-operative blood ammonia levels measured in bioengineered liver (BEL) implant recipient (n=3) and portal-caval shunt (n=2) over the duration of the experiment. FIG. 28E depicts Kaplan-Meier curves showing animal survival times within portal-caval shunt and bioengineered liver (BEL) implant groups. Symbols are matched to ammonia values in FIG. 28D. FIG. 28F depicts representative immunostaining bioengineered liver (BEL) tissue explanted 48 h post-implant demonstrating maintenance of CD31 and albumin expression. FIG. 28G depicts representative immunostaining bioengineered liver (BEL) tissue explanted 48 h post-implant demonstrating maintenance of CD31 and FAH expression.

FIG. 29 depicts H&E staining of a decellularized kidney seeded with HUVECs via perfusion while in solution. The arrow points to the glomerulus.

FIG. 30 depicts H&E staining of a decellularized kidney seeded with HUVECs via perfusion while suspended in air. The arrow points to the glomerulus.

FIG. 31 depicts ammonia clearance rates of revascularized livers seeded with hepatocytes via the hepatic vein, bile duct, dual seeding or portal vein. The actual number of seeded hepatocytes is defined in parentheses for each liver.

FIG. 32 depicts ammonia clearance rates of revascularized livers seeded with hepatocytes via the hepatic vein, bile duct, dual seeding (hepatic and bile duct) or portal vein. The actual number of seeded hepatocytes is defined in parentheses for each liver.

FIG. 33 depicts the patency of revascularized livers seeded with hepatocytes via (from left to right); the bile duct, the bile duct in a multistage seeding, the hepatic vein, the hepatic vein+bile duct, and the portal vein (PV). Patency was assessed utilizing a blood loop.

FIG. 34 depicts immunofluorescent staining of tri-culture grafts of bio-engineered liver with hepatic vein seeded hepatocytes.

FIG. 35 depicts immunofluorescent staining of tri-culture grafts of bio-engineered liver with hepatic vein seeded hepatocytes.

FIG. 36 depicts immunofluorescent staining of tri-culture grafts of bio-engineered liver with bile duct-seeded hepatocytes.

FIG. 37 depicts immunofluorescent staining of tri-culture grafts of bio-engineered liver with bile duct-seeded hepatocytes.

FIG. 38 depicts an exemplary schematic description of porcine kidney decellularization and representative gross, immunofluorescence, and histological images comparing native and decellularized kidneys. FIG. 38A depicts kidneys weighing from 250 to 300 grams recovered from adult pigs and decellularized according to a custom perfusion decellularization protocol. FIG. 38B shows decellularization caused a gross loss of color as the native porcine cells were solubilized and extracted, while the original size, shape, and structure of the kidney were well-retained in the decellularized scaffold (scale bars, 5 cm). FIG. 38C depicts immunofluorescence staining showing retention of collagen I, laminin, and collagen IV (green) and complete removal of nuclei (blue DAPI stain) (scale bars, 100 μm). FIG. 38D depicts high-magnification H&E images revealing the ECM structures left behind in decellularized kidneys, including blood vessels, glomeruli, nephron tubules, collecting ducts, and papillae (scale bars, 50 μm).

FIG. 39 depicts in vitro human endothelialization of decellularized porcine kidneys using a perfusion recellularization bioreactor system and correlation of glucose consumption kinetics with vascular endothelial cell coverage. FIG. 39A depicts a perfusion system used for kidney recellularization consisted of a reservoir holding a kidney, an air vent (AV), a pressure transducer (PT) to monitor perfusion pressure, a gas exchange coil (GEC) to allow for diffusion of gas into perfusate, a bubble trap (BT), and several three-way (3W) stopcocks to direct a flow of media to a kidney. Perfusion was driven by a peristaltic pump under control by custom perfusion software. FIG. 39B depicts total glucose consumption rate (GCR) by endothelial cells. This was calculated by monitoring the changes in glucose concentration in bioreactor media samples over time. Four distinct zones (1-4) were defined to distinguish endothelial cell coverage based on GCR and culture time. FIG. 39C depicts representative H&E images and immunofluorescence images revealed endothelialization of the vasculature and glomerular capillaries at each zone (Green: CD31 staining HUVECs, red: collagen I staining the porcine matrix; blue: DAPI staining cell nuclei).

FIG. 40 depicts an evaluation of endothelialized kidney performance in ex vivo blood loops. FIG. 40A depicts an ex vivo blood loop created by placing catheters in a carotid artery and jugular vein of an anesthetized adult pig. These catheter lines were respectively connected via Luer adapters to a renal artery and vein of an endothelialized kidney graft. FIG. 40B depicts a Transonic flow meter used to monitor volumetric flow rate of blood leading into a renal artery of an organ. Decellularized kidneys (no cells) or endothelialized grafts with Low GCR did not maintain blood flow beyond several minutes. Endothelialized grafts near Peak GCR maintained consistent perfusion throughout the entire study period (30 to 80 minutes). Quantified angiographic images showed a high percentage of graft perfusion. Data for perfusion was reported as mean+standard deviation and with individually plotted data points. FIG. 40C depicts representative Masson's Trichrome stain or CD31/Collagen I immunofluorescence images (asterisks denote the lumens of patent blood vessels; open arrows point to thrombosed blood vessels).

FIG. 41 depicts orthotopic transplantation of human endothelialized kidney grafts in a pig model. FIG. 41A depicts adult pigs that underwent unilateral nephrectomy followed by orthotopic transplantation of endothelialized kidney grafts. Anastomosed kidney graft depicted before and after reperfusion. FIG. 41B depicts a compilation of follow-up angiographies performed on a kidney graft through post-operative day 14. FIG. 41C depicts a percent perfusion quantified from angiographies performed on a day of surgery and through post-operative days 3, 7, 10, and 14 comparing high percent perfusion in patent grafts vs. loss of perfusion in thrombosed kidney grafts. At most follow-ups, percent patency was either consistent with the previous time point or had declined to near-zero due to thrombosis at the arterial inflow (n=1), venous outflow (n=1), or both vessels (n=1). Additionally, contralateral native kidneys (n=3) were analyzed for comparison and presented as the dashed line below. FIG. 41D depicts individual graft endpoints. FIG. 41E shows percent perfusion quantified from angiographies performed on the day of surgery and through POD 7. At most follow-ups, percent patency was either consistent with the previous time point or had declined to near-zero due to thrombosis at the arterial inflow (n=1) or venous outflow (n=1) likely from torsion. Additionally, contralateral native kidneys (n=3) were analyzed for comparison. FIG. 41F shows a Trichrome stain showing small (<100 um) blood vessels that remained patent at explant at regular follow-up time intervals following chronic orthotopic transplantation. CD31 stain shows endothelialized blood vessels with open arrows pointing to positive expression. Asterisks denote the lumens of patent blood vessels.

FIG. 42 depicts histological characterization of vascular patency in chronically transplanted endothelialized kidney grafts. Patent endothelialized kidney grafts were explanted at regular follow-up time intervals following chronic orthotopic transplantation in a pig model. Explanted grafts were flushed with saline, fixed, and processed for histological staining. Representative trichrome images show large (>250 μm) and small (<100 μm) blood vessels that remained patent and cleared of residual blood after post-explant flushing. Asterisks denote the lumens of patent blood vessels. Open arrows indicate occluded glomerular capillaries.

FIG. 43 depicts donor human endothelial cell turnover with recipient porcine endothelial cells in chronically transplanted endothelialized kidney grafts. Patent endothelialized kidney grafts were explanted at regular follow-up time intervals following chronic orthotopic transplantation in a pig model. Explanted grafts were flushed with saline, fixed, and processed for immunofluorescence staining. Endothelial cells were identified as donor (human) or recipient (porcine) in explanted kidney grafts using immunofluorescence staining. FIG. 43A depicts images showing the luminal surfaces of major blood vessels exceeding 500 μm in diameter. FIG. 43B depicts minor blood vessels less than 250 μm in diameter. Asterisks denote the lumens of patent blood vessels. White arrow outline indicates positive reactivity by rabbit CD31 antibody (rCD31; red stain), which reacts to both human and pig endothelial cells. Grey arrow fill indicates positive expression by mouse CD31 antibody (mCD31; green stain), which reacts positively only in human endothelial cells. Top row: rCD31/DAPI overlay. Middle row: mCD31/DAPI overlay. Bottom row: rCD31/mCD31/DAPI merged images. Orange shows colocalization of both antibodies, indicating human endothelial cells. The presence of residual blood causes faint background staining in some images. Donor human endothelial cells were completely absent from the graft by day 7, while new pig endothelialization started to occur at day 7 and continued through day 14. Scale bars, 100 μm.

FIG. 44 shows ammonia concentration as a function of time for 4 bioengineered livers dosed with excess ammonia to evaluate clearance as compared to control porcine livers. The BEL are able to clear the ammonia and support the use of the BEL for the treatment of acute liver failure as either a bridge to transplant or as a therapeutic.

FIG. 45 shows an exemplary approach to kidney-based bioengineering. Decellularization is used to remove cells from pig kidneys, producing a non-immunogenic matrix that serves as a biological scaffold for recellularization. Human donor kidneys discarded from the transplant pool are digested enzymatically to isolate human cells that are seeded into the decellularized pig matrix and cultured in vitro using perfusion bioreactors. Matured bioengineered kidneys (BEKs) are then ready for implantation to provide renal replacement therapy (RRT).

FIG. 46 shows preliminary glomerular co-culture and induction. FIG. 46A shows HUVEC-recellularized BEK at t=7 days after seeding. FIG. 46B shows Co-culture recellularized BEK (i.e., HUVECs and glomerular cells) at t=7 days after seeding. Asterisks denote stable capillary lumens, where more lumens were observed in the co-culture grafts. Scale bars: 50 μm. FIG. 46C and FIG. 46D are examples of human glomerular outgrowth cells in normal media with an epithelial morphology. Faint podocin expression is shown in FIG. 46C, with strong expression of a-catenin in FIG. 46D. FIG. 46E and FIG. 46F show human outgrowth cells after 7 days in induction media have arborized phenotype with stronger expression of podocin (red) and new expression of synaptopodin (green). Scale bars: 100 um.

FIG. 47A shows Kidney functional testing setup where a plasma analogue (KFT) solution is perfused through the kidney artery in physiological conditions in vitro. The system recirculates the KFT once the kidney is submerged from venous outflow. The ureter outflow is separately collected. FIG. 47B shows BSA retention in native versus decellularized kidneys (n=4) after kidney functional testing demonstrating protein retention only in native kidneys or recellularized BEKs.

FIG. 48A shows an in vivo blood loop schematic and gross images of a rBEK before and after reperfusion and imaging. FIG. 48B shows transonic flow measurements and angiography analysis. Decellularized kidneys (no cells) or rBEKs with low GCRs did not maintain blood flow beyond 5 min. rBEKs near Peak GCR maintained consistent perfusion throughout the entire 30 min. study period. Quantified angiographic images showed a high percentage of graft perfusion. FIG. 48C shows perfusion data reported as mean±standard deviation and with individually plotted data points. FIG. 48D shows Representative Masson's Trichrome stains (asterisks denote patent blood vessels; arrows point to thrombosed blood vessels). Scale bars: 50 um.

FIG. 49A shows levels of hematocrit (percent packed cell volume) for blood, HUVEC, and BEK samples of recellularized porcine kidneys tested using an ex vivo blood loop system. FIG. 49B shows levels of protein or creatine, normalized to blood, for native porcine kidney, native allograft (4 hrs. HTK), HUVEC only, or BEK samples of recellularized porcine kidneys tested using an ex vivo blood loop system. FIG. 49C shows urine, effluent flow rate for porcine native kidney, native allograft (4 hrs. HTK), HUVEC only, and BEK of recellularized porcine kidneys tested using an ex vivo blood loop system.

### DETAILED DESCRIPTION

Provided herein are methods to substantially decellularize isolated organs or portions thereof and methods of recellularizing the same. Also disclosed herein are methods providing improvements of recellularization and the resulting recellularized compositions. In an aspect, methods of improved recellularization disclosed herein may result in higher efficiency of recellularization and functionality of the compositions provided herein.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

The term “about” or “approximately” means within an acceptable error range for the particular value as determined by one of ordinary skill in the art, which will depend in part on how the value is measured or determined, i.e, the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell may not originate from a natural organism (e.g. a cell can be synthetically made, sometimes termed an artificial cell). Of particular interest are mammalian cells, from e.g., mammals including test animals and humans.

The term “substantially” as used herein may refer to a value approaching 100% of a given value. In some embodiments, the term may refer to an amount that may be at least about 90%, 91%, 92%, 93%, 94%, 95%, 96%, 97%, 98%, 99%, 99.9%, or 99.99% of a total amount. In some embodiments, the term may refer to an amount that may be about 100% of a total amount.

The term “decellularized” or “decellularization” as used herein may refer to a biostructure (e.g., an isolated organ or portion thereof, or tissue), from which the cellular and tissue content has been removed leaving behind an intact or substantially intact acellular infra-structure. Organs such as the kidney can be composed of various specialized tissues. Specialized tissue structures of an organ, or parenchyma, can provide specific function associated with the organ. Supporting fibrous network of an isolated organ can be a stroma. Most organs have a stromal framework composed of unspecialized connecting tissue which supports the specialized tissue. The process of decellularization may at least partially remove the cellular portion of the tissue, leaving behind a complex three-dimensional network of extracellular matrix (ECM). An ECM infrastructure may primarily be composed of collagen but can include cytokines, proteoglycans, laminin, fibrillin, endosomes, extracellular bound vesicles, and other proteins secreted by cells. An at least partially decellularized structure may provide a biocompatible substrate onto which different cell populations may be infused or used to be implanted as acellular medical devices such as but not limited to, wound care matrix, fistula matrix, void filler, dermal fillers, soft tissue reinforcement, or other substrates that enable cellular infiltration and remodeling following implantation or application. Decellularized biostructures may be rigid, or semi-rigid, having an ability to alter their shapes. Examples of decellularized isolated organs may include, but are not limited to solid organs such as, a heart, kidney, liver, lung, pancreas, brain, bone, spleen, and bladder, uterus, ureter, and urethra.

The term “effective amount” or “therapeutically effective amount” may refer to a quantity of a composition, for example a composition comprising cells such as regenerative cells, that can be sufficient to result in a desired activity upon introduction into an isolated organ or portion thereof disclosed herein.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose.

The term “recipient” and their grammatical equivalents as used herein may refer to a subject. A subject may be a human or non-human animal. A recipient may also be in need thereof, such as needing treatment for a disease such as cancer. In some embodiments, a recipient may be in need thereof of a preventative therapy. A recipient may not be in need thereof in other cases.

The term “subject” and its grammatical equivalents as used herein may refer to a human or a non-human. A subject may be a mammal. A subject may be a human mammal of a male or female biological gender. A subject may be of any age. A subject may be an embryo. A subject may be a newborn or up to about 100 years of age. A subject may be in need thereof. A subject may have a disease such as cancer. A subject may be premenopausal, menopausal, or have induced menopause.

The terms “treatment” or “treating” and their grammatical equivalents may refer to the medical management of a subject with an intent to cure, ameliorate, stabilize, or prevent a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include preventative treatment, that is, treatment directed to minimizing or partially or completely inhibiting the development of a disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure, ameliorate, stabilize or prevent a disease, condition, or disorder.

Disclosed herein are compositions and methods of whole organ decellularization as well as decellularization of whole organ portions. Also disclosed herein are improvements of decellularization utilizing methods and compositions. Also disclosed herein are compositions and methods of improved decellularization which may result in higher efficiency decellularization and improved recellularization of compositions described herein.

### Overview

Disclosed herein are methods, compositions, and systems for reconstituting an isolated at least partially decellularized organ with an endothelium. In some embodiments, compositions as disclosed herein can comprise part of a bioengineered organ. In some embodiments, a bioengineered organ can comprise a bioengineered kidney (BEK). In some embodiments, a bioengineered kidney can comprise a bioengineered organ comprise a bioengineered liver (BEL). In some embodiments, methods, compositions and systems herein can be used in a treatment for end-stage renal disease.

In some embodiments, a development of a bioengineered kidney can overcome challenges associated with end-stage renal disease by eliminating a need for kidney donation and eliminating a need for dialysis. In some embodiments, if a bioengineered kidney was developed using autologous cells, it would eliminate a need for life-long immunosuppression.

In some embodiments, a design criteria for development of a bioengineered kidney can include essential multifaceted filtration and regulatory systems that a kidney provides. In some embodiments, key functions of a kidney are to eliminate nitrogenous waste and to maintain blood pressure, volume, and composition. In some embodiments, a main functional unit of a kidney is a nephron and it consists of a glomerulus, which is an intricate globular network of blood vessels that provide efficient ultrafiltration of blood to excrete wastes, and tubular networks extending from a glomerulus to maintain fluid and electrolyte homeostasis. In some embodiments, a difficulty of designing a bioengineered kidney can be increased by a complexity of structural features of a kidney extracellular matrix (ECM) as well as its extracellular signaling cues. In some embodiments, extracellular signaling clue can be crucial for maintaining kidney function. In some embodiments, recapitulating a complex architecture and function of a kidney can be an extraordinarily challenging task.

In some embodiments, directed differentiation of pluripotent stem cells can be used to generate kidney organoids. In some embodiments, kidney organoids can contain nephrons and collecting duct structures. In some embodiments, organoid engineering can have tremendous potential for disease modeling, diagnostic drug toxicity screening, gene editing, and other applications. In some embodiments, organoids developed using these protocols lack a perfusable vasculature and a urinary drainage system, which limits their diameter to 1-2 cm and prevents them from recapitulating renal filtrative and excretory functions, and therefore precludes their translation to clinical therapies.

In some embodiments, perfusion decellularization of intact organs can address these technological hurdles, allowing for a generation of a three-dimensional (3D), acellular, and whole-organ scaffold at human-scale that maintains a complex architecture, vasculature, composition, and bioactivity of a native organ. In some embodiments, methods disclosed herein can be used to create a variety of whole-organ scaffolds. In some embodiments, whole-organ scaffolds can comprise heart, lung, liver, pancreas, or kidney. In some embodiments, decellularized kidney grafts can be recellularized. In some embodiments, decellularized kidney grafts can promote differentiation of pluripotent stem cells along a renal lineage. In some embodiments, decellularized kidney grafts can be used for transplantation in preclinical models.

In some embodiments, implanted acellular porcine kidney grafts implanted into a pig model for two weeks can be reperfused and sustain blood pressure without blood extravasation during surgery. In some embodiments, methods disclosed herein can at least partially prevent a graft exhibiting vascular thrombosis upon histological examination. In some embodiments, methods disclosed herein can at least partially prevent a decellularized kidney graft from being thrombosed 7 days after implantation into an animal model. In some embodiments, acute thrombogenic response in a notable study in which whole rat kidneys were decellularized, recellularized with both rat epithelial and human endothelial cells, and then perfused in a bioreactor where they produced rudimentary filtrate in vitro. In some embodiments, re-endothelializing an acellular graft can result in a graft showing no sign of thrombus formation in vivo, when implanted acutely in vivo in rats. In some embodiments, a length of time these grafts were studied in vivo was not reported and was achieved in a small animal model at a significantly smaller scale compared to human kidney transplantation.

In some embodiments, a recellularized kidney graft can remain patent with sustained perfusion of human whole blood for 25 minutes.

### Suitable Organs and Portions Thereof

Disclosed herein are compositions that can comprise isolated organs or portions thereof. Also disclosed herein are modified isolated organs or portions thereof. In some embodiments, an isolated organ or portion thereof may be part of an isolated organ system of a body. In some embodiments, isolated organ systems can include without limitation an integumentary, muscular, skeletal, nervous, circulatory, lymphatic, respiratory, endocrine, urinary/excretory, reproductive and digestive systems. In some embodiments, an isolated organ or portion thereof can be from a musculoskeletal system. In some embodiments, isolated organs or portions thereof from a musculoskeletal system can comprise skeleton, joints, ligaments, tendons, muscle. In some embodiments, an isolated organ or portion thereof can be from a digestive system. In some embodiments, a digestive system may comprise a tongue, tooth, salivary gland (parotid, submandibular, sublingual), pharynx, esophagus, stomach, small intestine (duodenum, jejunum, and ileum), large intestine, liver, gall bladder, or pancreas. In some embodiments, an isolated organ or portion thereof may comprise a respiratory system. In some embodiments, a respiratory system may comprise a lung, diaphragm, bronchi, trachea, larynx, pharynx, or nasal cavity. In some embodiments, an isolated organ or portion thereof may comprise at least a portion of a urinary system. In some embodiments, a urinary system may include a kidney, ureter, bladder, or urethra. In some embodiments, an isolated organ or portion thereof may comprise at least a portion of a reproductive system. In some embodiments, a reproductive system may comprise an ovary, fallopian tube, uterus, vagina, vulva, clitoris, placenta, testes, epididymis, vas deferens, seminal vesicle, prostate, bulbourethral gland, penis, or scrotum. In some embodiments, an isolated organ or portion thereof may include an endocrine gland. In some embodiments, an endocrine gland may comprise a pituitary gland, pineal gland, thyroid gland, parathyroid gland, adrenal gland, or pancreas. In some embodiments, an isolated organ or portion thereof may comprise at least a portion of a circulatory system. In some cases, an at least partially recellularized isolated organ or portion thereof is connected to a circulatory system via a cannula. In some embodiments, a circulatory system may comprise a cardiovascular system or a lymphatic system. In some embodiments, a cardiovascular system may comprise a heart, artery, vein, or capillary. In some embodiments, a lymphatic system may comprise a lymphatic vessel, a lymph node, bone marrow, thymus, or spleen. In some embodiments, an isolated organ or portion thereof may be from a nervous system. In some embodiments, a nervous system may comprise a central nervous system, peripheral nervous system, or sensory organ. In some embodiments, a central nervous system may comprise a brain, cerebrum, diencephalon, midbrain, pons, medulla oblongata, cerebellum, spinal cord, ventricular system (choroid plexus). In some embodiments, a peripheral nervous system may comprise nerves such as cranial nerves, spinal nerves, ganglia, enteric nervous system. In some embodiments, a sensory organ may comprise an eye (cornea, iris, ciliary body, lens, or retina), ear (earlobe, eardrum, middle ear, cochlea, semicircular canals, and vestibule of the ear), olfactory epithelium, or tongue. In some embodiments, an isolated organ or portion thereof may comprise at least a portion of an integumentary system. In some embodiments, an integumentary system may comprise mammary glands, skin, or subcutaneous tissue. In some embodiments, an isolated organ may be a solid organ. In some embodiments, a solid organ may have a firm tissue consistency, and may neither be hollow, such as organs of the gastrointestinal tract, nor liquid, such as blood. In some embodiments, a solid organ may include a heart, kidney, liver, lung, and pancreas. In some embodiments, an at least partially recellularized isolated organ or portion thereof can be selected from the group consisting of: an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, and an at least partially recellularized pancreas or portion thereof.

In an aspect, a solid organ or portion thereof may have three components: an extracellular matrix (ECM), cells embedded therein, and a vasculature bed.

In an aspect, an ECM can comprise a mixture of structural and practical proteins, lipids, proteoglycans, endosomes, and crystals. In an aspect, an ECM can promote cell aggregation, adhesion, migration, proliferation, maturation, functional maintenance, and differentiation in a way that reflects the functional necessities and biological uniqueness of tissues. Such guiding elements may be retained, at least in part, even in the lack of living cellular components, for example after decellularization of an organ or portion thereof. In an aspect, a decellularized ECM can be used as an off-the-shelf and immune-compatible alternative to living grafts for tissue and organ restoration. Decellularized ECM can stimulate regenerative processes, not only through specific organomorphic structures but also by its physiological introduction. The ECM can be involved in cell communication, as well as in defining the shape and stability of tissues. In some embodiments, the instructive scaffold materials derived from decellularized ECM can be activated by living cells prior to implantation, with the expectation that the ECM is capable of directing and or enhancing the differentiation fate of the seeded cells. During the process of tissue decellularization, preservation of the native ultrastructure and composition of the ECM is highly desirable.

In some embodiments, decellularization of a solid organ as described herein may remove most or all cellular components while substantially preserving an extracellular matrix (ECM) and a vasculature bed. In some embodiments, an at least partially decellularized isolated organ or portion thereof may comprise an extracellular matrix. In some embodiments, an extracellular matrix may comprise fibronectin, fibrillin, laminin, elastin, a collagen family protein, a glycosaminoglycan, a ground substance, a reticular fiber or thrombospondin. In some embodiments, a collagen family protein may comprise collagen I, collagen II, collagen III, or collagen IV. In some embodiments, an at least partially decellularized isolated organ or portion thereof may comprise a vasculature bed.

In some cases, a perfusion decellularized ECM can comprise a substantially intact exterior surface or an at least partially intact exterior surface. A perfusion decellularized ECM can comprise a decellularized ECM vascular tree that can include an intact vasculature bed. In some cases, a substantially intact vasculature can comprise a circulating fluid. In some cases, a circulating fluid can comprise blood or a fraction thereof. In some cases, a perfusion decellularized ECM can retain at least a portion of, or a majority of, a fluid introduced to a decellularized ECM vasculature tree.

In some embodiments, an isolated organ or portion thereof may be allogenic, autologous, or xenogeneic to a subject or to an origin of a cell. In some embodiments, an isolated organ or portion thereof may be from an adult or pediatric source. In some embodiments, an isolated organ or portion thereof may comprise synthetic components. In some embodiments, non-human isolated organ sources may be from non-human mammals. In some embodiments, non-human isolated organs may be from a non-human primate. In some embodiments, a non-human primate may be a baboon or chimpanzee. In some embodiments, a non-human isolated organ or portion thereof may be from a mammal including but not limited to a rodent, pig, rabbit, cattle, sheep, dog, cat, cow, or monkey. In some embodiments, an isolated organ or portion thereof may be human. An isolated organ or portion thereof from a human can be from a cadaver or from a living donor.

In some embodiments, a gnotobiotic mammal can be utilized to source an isolated organ or portion thereof. In some embodiments, a gnotobiotic mammal may be absent a microbial flora. In some embodiments, a gnotobiotic mammal may be surgically delivered under aseptic conditions. In some embodiments, a gnotobiotic mammal may be raised in an isolation type barrier. In some embodiments, a gnotobiotic mammal may be free of microbial flora from about 5%, 10%, 20%, 30%, 40%, 50%, 60%, 70%, 80%, 90%, 95%, 98%, 99%, 100% as compared to a comparable mammal that is not gnotobiotic. Exemplary zoonotic agents that a gnotobiotic mammal may be substantially devoid of may be rabies, monkey pox virus, Brucella suis, mycobacterium spp, Trypanosoma cruzi, Ascaris spp, larvae, Toxoplasma gondii, EMCV, measles, rubella, hepatocystiis kochi, influenza, African swine fever, swine vesicular disease virus, to name a few.

In some cases, an organ or portion thereof may be synthetic. Provided herein can be chimeric organs comprising partially synthetic and partially non-synthetic portions.

### Decellularization

Provided herein are methods of decellularizing organs and portions of organs. Also disclosed herein are compositions that can comprise decellularized isolated organs and methods of decellularization. In some embodiments, an isolated organ may undergo decellularization. In some embodiments, decellularization may refer to a process comprising isolating an extracellular matrix (ECM) of a tissue from its native cells. An at least partially decellularized isolated organ or tissue may comprise an extracellular matrix (ECM) component of all or most regions of an isolated organ or portion thereof, or tissue, including ECM components of a vascular tree. In some embodiments, an ECM component may include: fibronectin, fibrillin, laminin, elastin, members of the collagen family (e.g., collagen I, III, and IV), glycosaminoglycans, proteoglycans, cytokines, heparin sulfate, ground substance, reticular fibers, endosomes, ECM bound vesicles, or thrombospondin, or any combination thereof, which may remain organized as defined structures such as a basal lamina. In some embodiments, decellularization may refer to a reduction or absence of detectable myofilaments, endothelial cells, smooth muscle cells, and nuclei in histologic sections using standard histological staining procedures. Residual cell debris may also be removed from a decellularized isolated organ or tissue. Organ decellularization may be performed using a variety of techniques such as perfusion decellularization, immersion decellularization, physical treatments, chemical treatments, enzymatic treatments, and combinations thereof. In some cases, an isolated organ or portion thereof can be perfused with a perfusion solution.

### Perfusion Decellularization

Also disclosed herein are methods that can comprise perfusion-based decellularization of a solid organ or portion thereof. In some embodiments, perfusion decellularization can allow for decellularization through a native vasculature, ducts, cavities, and combinations thereof which may result in a rapid decellularization of large organs or portions thereof while maintaining vascular conduits critical to re-engineering of tissue intact. In some embodiments, perfusion decellularization can provide for rapid access to a whole organ through a native vasculature by cannulating a vasculature and perfusing a mild detergent solution through a native blood vessel. Because organs can be dense with vascular capillaries, most cells can be located within 50-100 micrometers (μm) of a capillary, resulting in an exponential increase in effective surface area of a detergent and decreased time to dissolve cellular material as it is expelled through a venous system, as opposed to through an isolated organ wall or capsule as is done in immersion-based decellularization. Perfusion decellularization of an ECM from an isolated organ or tissue may retain a native microstructure, such as an intact vascular and/or microvascular system, as compared to other decellularization techniques such as immersion based decellularization. For example, perfusion decellularized ECM from organs or tissues may preserve collagen content and other binding and signaling factors and vasculature structure, thus providing for a niche environment with native cues for functional differentiation or maintenance of cellular function of introduced cells. In some embodiments, perfusion decellularized ECM from organs or tissues may be perfused with cells and/or media using a vasculature of a perfusion decellularized ECM under appropriate conditions, including appropriate pressure and flow to mimic conditions normally found in an organism. A normal pressure of human sized organs may be between about 5 mm Hg to about 200 mm Hg with a resulting flow rate dependent upon an incoming perfusion vessel diameter. In some embodiments, a normal pressure of human sized organs may be from 5 mm Hg, 10 mm Hg, 15 mm Hg, 20 mmHg, 25 mmHg, 30 mmHg, 40 mmHg, 50 mmHg, 60 mmHg, 80 mmHg, 100 mmHg, 120 mmHg, 140 mmHg, 160 mmHg, 180 mmHg, or up to about 200 mmHg. For a normal human heart, a resulting perfusion flow can be about 20 mL/min/100 g to about 200 mL/min/100 g. Using such a system for seeded cells may achieve a greater seeding concentration of, for example, about 5× up to about 1000× greater than achieved under 2D cell culture conditions and, unlike a 2D culture system, an ECM environment allows for a further functional differentiation of cells, e.g., differentiation of progenitor cells into cells that demonstrate organ- or tissue-specific phenotypes having sustained function. Perfusion decellularization can comprise cannulating an isolated organ or portion thereof. In some embodiments, at least one cannulation can be introduced to an isolated organ or portion thereof. In some cases, at least two cannulations can be introduced to an isolated organ or portion thereof. In some embodiments, from about 1, 2, 3, 4, 5, 6, 7, 8, 9, or up to 10 cannulations can be introduced to an isolated organ or portion thereof. In some embodiments, a cannula may be a part of a cannulation system. In some embodiments, a cannulation system may comprise a size-appropriate hollow tubing for introducing into a vessel, duct, cavity, or any combination thereof of an isolated tissue, organ or portion thereof. In some embodiments, a cannula may be of an animal-appropriate diameter; for example, a cannula may be of a certain gauge. In some embodiments, gauges may range from 1 to 75. In some embodiments, a gauge may be of gauge 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75. In some embodiments, a cannula may have a gauge of about 16. In some embodiments, a cannula may have a gauge of about 23. In some embodiments, a cannula may have a gauge of about 27. In some embodiments, a cannula may have a gauge of about 30. In some embodiments, a cannula may have a gauge of about 34. In some embodiments, a cannula may have a gauge between 16 gauge and 34 gauge. In some embodiments, a cannula may have a gauge between 30 gauge and 50 gauge. In some embodiments, a cannula may have a gauge between 30 gauge and 50 gauge. In some embodiments, a type of “cannula” may vary. In some embodiments, a typical cannula used for blood draw or IV can be ˜14-26 gauge. In some embodiments, a cannula may comprise a barbed connector with a luer fitting or a two-way barbed tube to tube connector. In some embodiments, a barbed end may vary from about 1/32″ to about ⅝″ and greater; for example, a cannula size for a 6 month porcine liver may be ⅝″ diameter. In some embodiments, a pig may use a cannula from 1/16″ to about ¼″. In some embodiments, a cannula may have a gauge from about 1/16″ to about 1″. In some embodiments, an isolated organ or portion thereof can comprise a cannula of a gauge of at least 34. In some embodiments, disclosed herein may be an isolated organ or portion thereof comprising a cannula of a gauge of at least 50. In some embodiments, disclosed herein may be an isolated organ or portion thereof comprising a cannula of a gauge of at least 16. In some embodiments, disclosed herein may be an isolated organ or portion thereof comprising a cannula of a gauge of at least 20. In some embodiments, at least one vessel, duct, and/or cavity may be cannulated in an isolated organ. In some embodiments, a perfusion apparatus or cannulation system may include a holding container for solutions (e.g., a cellular disruption medium) and a mechanism for moving a liquid through an isolated organ (e.g., a pump, air pressure, gravity) via one or more cannulae. In some embodiments, a cellular disruption media may be pumped by a peristaltic pump. In some embodiments, a cellular disruption media may be passed through an at least partly permeable tubing allowing gaseous exchange. In some embodiments, gaseous exchange may comprise oxygen exchange, nitrogen exchange, carbon dioxide exchange, or any combination thereof. In some embodiments, an at least partly permeable tubing may comprise silicone tubing.

In some embodiments, an isolated organ or tissue during decellularization and/or recellularization may be maintained in a sterile state using a variety of techniques such as: controlling and filtering air flow and/or perfusing with, for example, antibiotics, anti-fungals or other anti-microbials to prevent growth of unwanted microorganisms. In some embodiments, a system as described herein may possess an ability to monitor certain perfusion characteristics (e.g., pressure, volume, flow pattern, temperature, gases, pH), mechanical forces (e.g., ventricular wall motion and stress), and electrical stimulation (e.g., pacing). In some embodiments, a vascular bed may change during decellularization and recellularization (e.g., vascular resistance, volume). In some embodiments, a pressure-regulated perfusion apparatus or cannulation system may be advantageous to avoid or reduce fluctuations. In some embodiments, an effectiveness of perfusion may be evaluated in an effluent and in tissue sections. In some embodiments, perfusion volume, flow pattern, temperature, partial O₂ and CO₂ pressures and pH may be monitored using standard methods. In some embodiments, sensors may be used to monitor a system (e.g., a bioreactor) and/or an isolated tissue, organ or portion thereof. In some embodiments, sonomicrometry, micromanometry, and/or conductance measurements may be used to acquire pressure-volume or preload recruitable stroke work information relative to myocardial wall motion and performance. In some embodiments, for example, sensors may be used to monitor a pressure of a liquid moving through a cannulated organ or tissue; an ambient temperature in a system and/or a temperature of an isolated organ or tissue; a pH and/or a rate of flow of a liquid moving through a cannulated organ or tissue; and/or a biological activity of a recellularizing organ or tissue. In some embodiments, in addition to having sensors for monitoring such features, a system for decellularizing and/or recellularizing an isolated organ or tissue also may include means for maintaining or adjusting such features. In some embodiments, means for maintaining or adjusting such features may include components such as a thermometer, a thermostat, electrodes, pressure sensors, overflow valves, valves for changing a rate of flow of a liquid, valves for opening and closing fluid connections to solutions used for changing a pH of a solution, a balloon, an external pacemaker, and/or a compliance chamber. In some embodiments, to help ensure stable conditions (e.g., temperature), a chamber, reservoir, or tubing may be water-jacketed. In some embodiments, a method of decellularization can comprise providing an isolated organ or portion thereof, cannulating an isolated organ or portion thereof, and perfusing a cannulated isolated organ or portion thereof with a solution or medium via a cannulation. In some embodiments, a cannulation occurs at a cavity, vessel, duct, or combination thereof. In some embodiments, from about 1 to 3, from about 1 to 5, from about 2 to 3, from about 2 to 5, from about 1 to 8 solutions may be utilized for organ perfusion. In some embodiments, a solution can be perfused at least two times. In some embodiments, a solution can be perfused at least 3, 4, 5, 6, 7, 8, 9, or 10 times through an isolated organ or portion thereof. In some embodiments, various solutions and mediums may be employed during recellularization. In some embodiments, a solution may be selected from a group comprising: cellular disruption media, washing solutions, disinfecting solutions, or combinations thereof. In some embodiments, a cellular disruption media can be a solution that may comprise at least one detergent, Table 1.

TABLE 1
Detergents that may be utilized in media, solutions, or fluids provided herein

| | | | Agg.# (No. | | | | |
|---|---|---|---|---|---|---|---|
| | | | of | MW | CMC | Cloud | |
| | | | molecules | mono | mM | point | |
| Detergent | Chemical name | Type | per micelle) | (micelle) | (% w/v) | ° C. | Dialyzable |
| Triton | Polyethylene | Nonionic | 140 | 647 | 0.24 | 64 | No |
| X-100 | glycol p-(1,1,3,3- | | | (90K) | (0.0155) | | |
| | tetramethylbutyl)- | | | | | | |
| | phenyl ether | | | | | | |
| Triton | 1,1,3,3- | Nonionic | — | 537 (-) | 0.21 | 23 | No |
| X-114 | Tetramethylbutyl) | | | | (0.0113) | | |
| | phenyl- | | | | | | |
| | polyethylene | | | | | | |
| | glycol | | | | | | |
| NP-40 | 4-Nonylphenol, | Nonionic | 149 | 617 | 0.29 | 80 | No |
| | branched, | | | (90K) | (0.0179) | | |
| | ethoxylated | | | | | | |
| Brij-35 | Polyoxyethylene | Nonionic | 40 | 1225 | 0.09 | >100 | No |
| | lauryl ether | | | (49K) | (0.0110) | | |
| Brij-58 | Polyethylene | Nonionic | 70 | 1120 | 0.08 | >100 | No |
| | glycol hexadecyl | | | (82K) | (0.0086) | | |
| | ether | | | | | | |
| Tween | Polyethylene | Nonionic | — | 1228 | 0.06 | 95 | No |
| 20 | glycol sorbitan | | | (-) | (0.0074) | | |
| | monolaurate | | | | | | |
| Tween | Polyethylene | Nonionic | 60 | 1310 | 0.01 | — | No |
| 80 | glycol sorbitan | | | (76K) | (0.0016) | | |
| | monooleate | | | | | | |
| Octyl | n-octyl-β-D- | Nonionic | 27 | 292 | 23-24 | >100 | Yes |
| glucoside | glucoside | | | (8K) | (~0.70) | | |
| Octyl | Octyl β-D-1- | Nonionic | — | 308 (-) | 9 | >100 | Yes |
| thioglucoside | thioglucopyranoside | | | | (0.2772) | | |
| Sodium | Dodecyl sodium | Anionic | 62 | 288 | 6-8 | >100 | No |
| dodecyl | sulfate | | | (18K) | (0.17-0.23) | | |
| sulfate | | | | | | | |
| CHAPS | 3-[(3- | Zwitterionic | 10 | 615 | 8-10 | >100 | Yes |
| | Cholamidopropyl) | | | (6K) | (0.5-0.6) | | |
| | dimethylammonio]- | | | | | | |
| | 1-propanesulfonate | | | | | | |
| | hydrate | | | | | | |
| CHAPSO | 3-([3- | Zwitterionic | 11 | 631 | 8-10 | 90 | Yes |
| | Cholamidopropyl] | | | (7K) | (~0.505) | | |
| | dimethylammonio)- | | | | | | |
| | 2-hydroxy-1- | | | | | | |
| | propanesulfonate | | | | | | |

In some embodiments, at least one of: Sodium dodecyl sulfate (SDS), Polyethylene glycol p-(1,1,3,3-tetramethylbutyl)-phenyl ether (Triton X), NP-40, Brij, Polyoxyethylene sorbitan monolaurate (Tween), Octyl glucoside, octyl thioglucoside, 3-[(3-cholamidopropyl)dimethylammonio]-1-propanesulfonate (CHAPS), 3-[(3-Cholamidopropyl)dimethylammonio]-2-hydroxy-1-propanesulfonate (CHAPSO), salts thereof, or modified versions thereof may be utilized during decellularization. In some embodiments, a detergent may be an amphipathic molecule that may contain both a nonpolar “tail” having aliphatic or aromatic character and a polar “head”. In some embodiments, an ionic character of a polar head group may form a basis for broad classification of detergents; they may be ionic (charged, either anionic or cationic), nonionic (uncharged), or zwitterionic (having both positively and negatively charged groups but with a net charge of zero). In some embodiments, detergents may be denaturing or non-denaturing with respect to protein structure. In some embodiments, denaturing detergents may be anionic such as sodium dodecyl sulfate (SDS) or cationic such as ethyl trimethyl ammonium bromide (ETMAB). In some embodiments, these detergents totally disrupt membranes and denature proteins by breaking protein-protein interactions. In some embodiments, non-denaturing detergents may be divided into nonionic detergents such as Triton X-100, NP40, Tween, bile salts such as cholate, and zwitterionic detergents such as CHAPS. In some embodiments, an at least partially degassed liquid solution may be utilized during decellularization. In some embodiments, a washing solution may be utilized to remove residual solutions such as cellular disruption media from an isolated organ or portion thereof as well as residual cellular components, enzymes, or combinations thereof. In some embodiments, suitable washing solutions may comprise water, filtered water, normal saline, phosphate buffered saline (PBS), or any combination thereof. In some embodiments, PBS may maintain a constant pH and constant osmolarity of cells (a pH of most biological materials falls from about 7 to about 7.6). In some embodiments, any concentration of PBS may be utilized as a washing solution. In some embodiments, a concentration of PBS may be about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, 55%, 60%, 65%, 70%, 75%, 80%, 85%, 90%, 95%, or up to about 100%. In some embodiments, a washing solution may be supplemented with agents. In some embodiments, an agent may be an antibiotic, DNase I, or a disinfectant. In some embodiments, a washing solution may comprise saline. In some embodiments, a washing solution may be 0.9% NaCl, 0.55% PBS, and 100% PBS, saline, and any combination thereof. In some embodiments, a cellular disruption media may be a degassed cellular disruption media. In some embodiments, a “degassed” cellular disruption media may refer to a disruption media as described herein, where a dissolved gas has been substantially removed. In some embodiments, a dissolved gas may include oxygen, nitrogen, air, carbon dioxide, carbon monoxide, argon, hydrogen sulfide, methane, ethylene, ethane, chlorine, hydrogen, helium, ammonia, sulfur dioxide, or any combination thereof. In some embodiments, a degassed media may contain less dissolved gases than a non-degassed media. In some embodiments, a degassed media may contain about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 99.9% of a dissolved gas content of a non-degassed media. In some embodiments, a degassed media may contain an amount of a gas at a given temperature; for example, a degassed media may contain less than about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, or 100 ppb of a gas at 25° C. In some embodiments, a degassed media may contain less than about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 ppm of a gas at 25° C. In some embodiments, a degassed media may be prepared by placing a media under reduced pressure (e.g. under a vacuum or a vacuum pump). In some embodiments, a “reduced pressure” may be a pressure less than an atmospheric pressure; for instance, a “reduced pressure” may be a pressure less than 760 torr. In some embodiments, a reduced pressure may be a vacuum, a medium vacuum, a high vacuum, or an ultra-high vacuum. In some embodiments, a reduced pressure may be a pressure less than about 760, 759, 758, 757, 756, 755, 754, 753, 752, 751, 750, 749, 748, 747, 746, 745, 744, 743, 742, 741, 740, 739, 738, 737, 736, 735, 734, 733, 732, 731, 730, 729, 728, 727, 726, 725, 724, 723, 722, 721, 720, 719, 718, 717, 716, 715, 714, 713, 712, 711, 710, 709, 708, 707, 706, 705, 704, 703, 702, 701, 700, 699, 698, 697, 696, 695, 694, 693, 692, 691, 690, 689, 688, 687, 686, 685, 684, 683, 682, 681, 680, 679, 678, 677, 676, 675, 674, 673, 672, 671, 670, 669, 668, 667, 666, 665, 664, 663, 662, 661, 660, 659, 658, 657, 656, 655, 654, 653, 652, 651, 650, 649, 648, 647, 646, 645, 644, 643, 642, 641, 640, 639, 638, 637, 636, 635, 634, 633, 632, 631, 630, 629, 628, 627, 626, 625, 624, 623, 622, 621, 620, 619, 618, 617, 616, 615, 614, 613, 612, 611, 610, 609, 608, 607, 606, 605, 604, 603, 602, 601, 600, 599, 598, 597, 596, 595, 594, 593, 592, 591, 590, 589, 588, 587, 586, 585, 584, 583, 582, 581, 580, 579, 578, 577, 576, 575, 574, 573, 572, 571, 570, 569, 568, 567, 566, 565, 564, 563, 562, 561, 560, 559, 558, 557, 556, 555, 554, 553, 552, 551, 550, 549, 548, 547, 546, 545, 544, 543, 542, 541, 540, 539, 538, 537, 536, 535, 534, 533, 532, 531, 530, 529, 528, 527, 526, 525, 524, 523, 522, 521, 520, 519, 518, 517, 516, 515, 514, 513, 512, 511, 510, 509, 508, 507, 506, 505, 504, 503, 502, 501, 500, 499, 498, 497, 496, 495, 494, 493, 492, 491, 490, 489, 488, 487, 486, 485, 484, 483, 482, 481, 480, 479, 478, 477, 476, 475, 474, 473, 472, 471, 470, 469, 468, 467, 466, 465, 464, 463, 462, 461, 460, 459, 458, 457, 456, 455, 454, 453, 452, 451, 450, 449, 448, 447, 446, 445, 444, 443, 442, 441, 440, 439, 438, 437, 436, 435, 434, 433, 432, 431, 430, 429, 428, 427, 426, 425, 424, 423, 422, 421, 420, 419, 418, 417, 416, 415, 414, 413, 412, 411, 410, 409, 408, 407, 406, 405, 404, 403, 402, 401, 400, 399, 398, 397, 396, 395, 394, 393, 392, 391, 390, 389, 388, 387, 386, 385, 384, 383, 382, 381, 380, 379, 378, 377, 376, 375, 374, 373, 372, 371, 370, 369, 368, 367, 366, 365, 364, 363, 362, 361, 360, 359, 358, 357, 356, 355, 354, 353, 352, 351, 350, 349, 348, 347, 346, 345, 344, 343, 342, 341, 340, 339, 338, 337, 336, 335, 334, 333, 332, 331, 330, 329, 328, 327, 326, 325, 324, 323, 322, 321, 320, 319, 318, 317, 316, 315, 314, 313, 312, 311, 310, 309, 308, 307, 306, 305, 304, 303, 302, 301, 300, 299, 298, 297, 296, 295, 294, 293, 292, 291, 290, 289, 288, 287, 286, 285, 284, 283, 282, 281, 280, 279, 278, 277, 276, 275, 274, 273, 272, 271, 270, 269, 268, 267, 266, 265, 264, 263, 262, 261, 260, 259, 258, 257, 256, 255, 254, 253, 252, 251, 250, 249, 248, 247, 246, 245, 244, 243, 242, 241, 240, 239, 238, 237, 236, 235, 234, 233, 232, 231, 230, 229, 228, 227, 226, 225, 224, 223, 222, 221, 220, 219, 218, 217, 216, 215, 214, 213, 212, 211, 210, 209, 208, 207, 206, 205, 204, 203, 202, 201, 200, 199, 198, 197, 196, 195, 194, 193, 192, 191, 190, 189, 188, 187, 186, 185, 184, 183, 182, 181, 180, 179, 178, 177, 176, 175, 174, 173, 172, 171, 170, 169, 168, 167, 166, 165, 164, 163, 162, 161, 160, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 torr. In some embodiments, a reduced pressure may be a pressure less than about 1×10⁰, 1×10⁻¹, 1×10⁻², 1×10⁻³, 1×10⁻⁴, 1×10⁻⁵, 1×10⁻⁶, 1×10⁻⁷, 1×10⁻⁸, 1×10⁻⁹, 1×10⁻¹⁰, 1×10⁻¹¹, or 1×10⁻¹² torr. In some embodiments, a degassed media may be prepared by heating a degassed media. In some embodiments, a degassed media may be placed at a temperature of about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. In some embodiments, a degassed media may be placed at a temperature of from about 25° C. to about 27° C., from about 25° C. to about 28° C., from about 25° C. to about 29° C., from about 25° C. to about 30° C., from about 25° C. to about 31° C., from about 25° C. to about 32° C., from about 25° C. to about 33° C., from about 25° C. to about 34° C., from about 25° C. to about 35° C., from about 25° C. to about 36° C., from about 25° C. to about 37° C., from about 25° C. to about 38° C., from about 25° C. to about 39° C., from about 25° C. to about 40° C., from about 25° C. to about 41° C., from about 25° C. to about 42° C., from about 25° C. to about 43° C., from about 25° C. to about 44° C., from about 25° C. to about 45° C., from about 25° C. to about 46° C., from about 25° C. to about 47° C., from about 25° C. to about 48° C., from about 25° C. to about 49° C., from about 25° C. to about 50° C., from about 25° C. to about 51° C., from about 25° C. to about 52° C., from about 25° C. to about 53° C., from about 25° C. to about 54° C., from about 25° C. to about 55° C., from about 25° C. to about 56° C., from about 25° C. to about 57° C., from about 25° C. to about 58° C., from about 25° C. to about 59° C., from about 25° C. to about 60° C., from about 25° C. to about 61° C., from about 25° C. to about 62° C., from about 25° C. to about 63° C., from about 25° C. to about 64° C., from about 25° C. to about 65° C., from about 25° C. to about 66° C., from about 25° C. to about 67° C., from about 25° C. to about 68° C., from about 25° C. to about 69° C., from about 25° C. to about 70° C., from about 25° C. to about 71° C., from about 25° C. to about 72° C., from about 25° C. to about 73° C., from about 25° C. to about 74° C., from about 25° C. to about 75° C., from about 25° C. to about 76° C., from about 25° C. to about 77° C., from about 25° C. to about 78° C., from about 25° C. to about 79° C., from about 25° C. to about 80° C., from about 25° C. to about 81° C., from about 25° C. to about 82° C., from about 25° C. to about 83° C., from about 25° C. to about 84° C., from about 25° C. to about 85° C., from about 25° C. to about 86° C., from about 25° C. to about 87° C., from about 25° C. to about 88° C., from about 25° C. to about 89° C., from about 25° C. to about 90° C., from about 25° C. to about 91° C., from about 25° C. to about 92° C., from about 25° C. to about 93° C., from about 25° C. to about 94° C., from about 25° C. to about 95° C., from about 25° C. to about 96° C., from about 25° C. to about 97° C., from about 25° C. to about 98° C., from about 25° C. to about 99° C., from about 25° C. to about 100° C. In some embodiments, a degassed media may be placed at a temperature of from about 50° C. to about 52° C., from about 50° C. to about 53° C., from about 50° C. to about 54° C., from about 50° C. to about 55° C., from about 50° C. to about 56° C., from about 50° C. to about 57° C., from about 50° C. to about 58° C., from about 50° C. to about 59° C., from about 50° C. to about 60° C., from about 50° C. to about 61° C., from about 50° C. to about 62° C., from about 50° C. to about 63° C., from about 50° C. to about 64° C., from about 50° C. to about 65° C., from about 50° C. to about 66° C., from about 50° C. to about 67° C., from about 50° C. to about 68° C., from about 50° C. to about 69° C., from about 50° C. to about 70° C., from about 50° C. to about 71° C., from about 50° C. to about 72° C., from about 50° C. to about 73° C., from about 50° C. to about 74° C., from about 50° C. to about 75° C., from about 50° C. to about 76° C., from about 50° C. to about 77° C., from about 50° C. to about 78° C., from about 50° C. to about 79° C., from about 50° C. to about 80° C., from about 50° C. to about 81° C., from about 50° C. to about 82° C., from about 50° C. to about 83° C., from about 50° C. to about 84° C., from about 50° C. to about 85° C., from about 50° C. to about 86° C., from about 50° C. to about 87° C., from about 50° C. to about 88° C., from about 50° C. to about 89° C., from about 50° C. to about 90° C., from about 50° C. to about 91° C., from about 50° C. to about 92° C., from about 50° C. to about 93° C., from about 50° C. to about 94° C., from about 50° C. to about 95° C., from about 50° C. to about 96° C., from about 50° C. to about 97° C., from about 50° C. to about 98° C., from about 50° C. to about 99° C., or from about 50° C. to about 100° C. In some embodiments, a media may be placed under reduced pressure, under elevated pressure, or both, for a given amount of time to reduce a concentration of dissolved gas. In some embodiments, a media may be placed under reduced pressure, under elevated pressure, or both for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes. In some embodiments, a media may be placed under reduced pressure, under elevated pressure, or both for at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 hours. In some embodiments, a degassed media (e.g. a degassed decellularization or recellularization solution) may perform more efficiently than a non-degassed media when perfused into an isolated organ; for example, decellularization using a degassed media may require less volume of a degassed media relative to a non-degassed media to achieve a comparable amount of decellularization. In some embodiments, decellularization using a degassed media may require less time to achieve a given amount of decellularization relative to an amount of time required to achieve a comparable amount of decellularization using a non-degassed media. In some embodiments, perfusion with a degassed media produces fewer microbubbles than perfusion with a non-degassed media. In some embodiments, a disinfecting solution may be utilized during decellularization. In some embodiments, a disinfecting solution may comprise any number of agents such as antibiotics, disinfectants, or combinations thereof. In some embodiments, an antibiotic that may be used in a decellularization solution may be selected from a group comprising: actinomycin, ampicillin, carbenicillin, cefotaxime, fosmidomycin, gentamicin, kanamycin, neomycin, amphotericin, penicillin, polymyxin, streptomycin, broad selection antibiotic, and combinations thereof. In some embodiments, any concentration of antibiotic may be introduced in a disinfecting solution. In some embodiments, suitable concentrations of antibiotics may be: 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, or up to about 60%. In some embodiments, suitable concentrations of antibiotics may be: 0.5 U/ml, 1 U/ml, 5 U/ml, 10 U/ml, 20 U/ml, 30 U/ml, 40 U/ml, 50 U/ml, 60 U/ml, 70 U/ml, 80 U/ml, 90 U/ml, 100 U/ml, 110 U/ml, 120 U/ml, 130 U/ml, 140 U/ml, 150 U/ml, 160 U/ml, 170 U/ml, 180 U/ml, 190 U/ml, 200 U/ml, 300 U/ml, 400 U/ml, 500 U/ml, 600 U/ml, 700 U/ml, 800 U/ml, 900 U/ml, 1000 U/ml, and up to about 1500 U/ml. In some embodiments, suitable concentrations of antibiotics may be: 0.5 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml, 5.5 μg/ml, 6 μg/ml, 6.5 μg/ml, 7 μg/ml, 7.5 μg/ml, 8 μg/ml, 8.5 μg/ml, 9 μg/ml, 9.5 μg/ml, 10 μg/ml, 15 μg/ml, 20 μg/ml, g/ml, 30 μg/ml, 35 μg/ml, 40 μg/ml, 45 μg/ml, 50 μg/ml, or up to about 60 μg/ml. In some embodiments, an antibiotic may be 1% benzalkonium chloride, 100 U/ml penicillin-G, 100 U/ml streptomycin, 50 μg/ml Gentamicin and 0.25 μg/ml Amphotericin B. In some embodiments, generally, moderate concentrations of mild (i.e, nonionic) detergents may compromise cell membrane integrity, thereby facilitating lysis of cells and extraction of soluble protein, often in native form. In some embodiments, using certain buffer conditions, various detergents effectively penetrate between a membrane bilayer at a concentration sufficient to form mixed micelles with isolated phospholipids and membrane proteins. In some embodiments, denaturing detergents such as SDS may bind to both membrane (hydrophobic) and non-membrane (water-soluble, hydrophilic) proteins at concentrations below a CMC (i.e, as monomers). In some embodiments, a reaction may be equilibrium driven until saturated, therefore, a free concentration of monomers may determine a detergent concentration. In some embodiments, SDS binding can be cooperative (i.e, a binding of one molecule of SDS increases a probability that another molecule of SDS will bind to that protein) and alters most proteins into rigid rods whose length can be proportional to molecular weight. In some embodiments, non-denaturing detergents such as Triton X-100 have rigid and bulky nonpolar heads that do not penetrate into water-soluble proteins; consequently, they generally do not disrupt native interactions and structures of water-soluble proteins and do not have cooperative binding properties. In some embodiments, a main effect of non-denaturing detergents can be to associate with hydrophobic parts of membrane proteins, thereby conferring miscibility to them.

In some embodiments, an isolated organ or portion thereof is at least partially submerged in solution during perfusion or an introducing step. In some embodiments, an isolated organ or portion thereof is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% submerged in solution during perfusion. In some embodiments, disclosed herein is an at least partially recellularized isolated organ or portion thereof in which the recellularization occurred while the organ was at least partially submerged in a liquid. In some embodiments, an organ at least partially recellularized while submerged comprises advantageous properties in comparison to a comparable at least partially recellularized isolated organ or portion thereof which was not at least partially submerged during recellularization. In some embodiments, advantageous properties may comprise: a more even distribution of cellular deposition during recellularization, a greater number of cells attaching during recellularization, improved function of the isolated organ or portion thereof, or any combination thereof. In some embodiments, an at least partially recellularized isolated organ or portion thereof can comprise a population of engrafted exogenous cells, wherein a density of a population of exogenous cells in a distal portion of an at least partially recellularized isolated organ or portion thereof can comprises at most about a 10%, about a 20%, about a 30%, about a 40%, about a 50%, about a 60%, about a 70%, about an 80%, about a 90%, about a 100%, about a 150%, or about a 200% difference as compared to a density of a population of exogenous cells in a proximal portion of an at least partially recellularized isolated organ or portion thereof. In some cases, a density of a population of exogenous cells in a distal portion of an at least partially recellularized isolated organ or portion thereof as compared to a density of a population of exogenous cells in a proximal portion of an at least partially recellularized isolated organ or portion thereof can be measured by hematoxylin and eosin (H&E) staining of a population of exogenous cells in a distal portion and a proximal portion of an at least partially recellularized isolated organ or portion thereof.

In some embodiments, perfusion decellularization of an isolated organ or portion thereof may be from about 5%, 10%, 20%, 30%, 40%, 50%, 60%, 70%, 80%, 90%, 95%, or up to about 100% more effective as compared to a non-perfusion based decellularization system. In some embodiments, decellularization of an isolated organ or portion thereof may be determined using various means. In some embodiments, decellularization may be determined by histological examination. In some embodiments, histological examination may demonstrate a lack or reduction of cellular material, nuclei, and combinations thereof within an at least partially decellularized isolated organ or portion thereof with preservation of an overall structure such as lobules and central veins. In some embodiments, decellularization may be determined by immunohistochemical staining. In some embodiments, immunohistochemical staining may demonstrate paucity of cellular factors such as galactosyl-alpha (1,3) galactose (alpha-Gal) following perfusion decellularization. In some embodiments, decellularization may be determined using DNA quantification. In some embodiments, DNA quantification may comprise assays such as PicoGreen, qPCR, and Quant-IT. In some embodiments, DNA quantification assays may determine an amount of a reduction of DNA in an isolated organ or portion thereof. In some embodiments, decellularization may be determined by any of the methods of: histology with H&E staining, immunofluorescence for cellular structures, Immunohistochemical staining for cell membrane associated proteins, nuclear staining such as DAPI, or any combination thereof. In some embodiments, a perfusion-based decellularized isolated organ or portion thereof preserves a native scaffold containing an appropriate microenvironment required for an introduction of organ-specific cells, along with an intact vascular network to reconnect to a subject's blood supply and an outer capsule capable of maintaining physiologic pressures. In some embodiments, these components can be important for a later use of perfusion recellularization, which also uses perfusion to repopulate vascular and organ-specific regenerative cells onto an at least partially decellularized isolated organ or portion thereof, where they may migrate to an appropriate microenvironment (via a relevant signaling protein markers that remain within a perfusion decellularized scaffold) as an at least partially decellularized isolated organ or portion thereof grows and matures in a bioreactor under normal physiologic conditions. In some embodiments, a resulting at least partially recellularized isolated organ or portion thereof may then be transplanted utilizing a technique comparable to current organ transplantation. In some embodiments, scaffolds created by perfusion decellularization can be capable of receiving and incorporating a variety of cells. In some embodiments, a decellularization process may generate particulate. In some embodiments, particulate may refer to residual components from a decellularization. In some embodiments, particulate may be formed by an insoluble interaction between native proteins and detergents. In some embodiments, particulate may be immiscible in aqueous solution. In some embodiments, a presentation of a non-soluble white particulate may be seen during decellularization of whole organs with SDS or other detergents. In some embodiments, particulate forms and then becomes trapped in an organ during decellularization, which may negatively affect a use of a decellularized matrix for acellular products or affect use of a decellularized isolated organ portion thereof or matrix thereof for recellularization. In some embodiments, a method may reduce particulate formation by a use of saline solutions, non-fasting mammals, and a combination thereof. In some embodiments, reducing particulate formation may decrease cytotoxicity of a decellularized matrix to introduced cells, for instance during recellularization. In some embodiments, reducing particulate may be performed by using solutions, controlling a mammal's eating habits, or their combination.

### Immersion Decellularization

Also disclosed herein are methods of immersion-based decellularization of an isolated organ or portion thereof. In some embodiments, whole organs or portions thereof may be decellularized by removing an entire cellular and tissue content from an organ. In some embodiments, decellularization may comprise a series of sequential extractions. In some embodiments, a first step may involve removal of cellular debris and solubilization of a cell membrane. In some embodiments, this may be followed by solubilization of a nuclear cytoplasmic component and a nuclear component. In some embodiments, an isolated organ may be decellularized by removing a cell membrane and cellular debris surrounding an isolated organ using gentle mechanical disruption methods. In some embodiments, a gentle mechanical disruption method may disrupt a cellular membrane. In some embodiments, a process of decellularization may avoid damage or disturbance of a biostructure's complex infra-structure. In some embodiments, gentle mechanical disruption methods may include scraping a surface of an isolated organ or portion thereof, agitating an isolated organ or portion thereof, or stirring an isolated organ or portion thereof in a suitable volume of fluid, e.g., distilled water. In some embodiments, a gentle mechanical disruption method may include magnetically stirring (e.g., using a magnetic stir bar and a magnetic plate) an isolated organ or portion thereof in a suitable volume of distilled water until a cell membrane can be disrupted and a cellular debris has been removed from an isolated organ or portion thereof. In some embodiments, after a cell membrane has been removed, a nuclear or cytoplasmic biostructure component can be removed. In some embodiments, this may be performed by solubilizing cellular or nuclear components without disrupting an infra-structure. In some embodiments, to solubilize a nuclear component, non-ionic detergents or surfactants may be used. In some embodiments, examples of nonionic detergents or surfactants include, but are not limited to, a Triton series surfactant, available from Rohm and Haas of Philadelphia, Pa., which includes Triton X-100, Triton N-101, Triton X-114, Triton X-405, Triton X-705, and Triton DF-16, available commercially from many vendors; a Tween series surfactant, such as monolaurate (Tween 20), monoamidite (Tween 40), monooleate (Tween 80), and polyoxethylene-23-lauryl ether (Brij. 35), polyoxyethylene ether W-1 (Polyol), sodium cholate, deoxycholates, CHAPS, saponin, n-Deacyl β-D-glucopyranoside, n-heptyl 3-D glucopyranoside, n-Octylα-D-glucopyranoside, Nonidet P-40, or any combination thereof.

### Physical Treatments

Also disclosed herein are methods of at least partial decellularization by means of physical treatment of an isolated organ or portion thereof. In some embodiments, physical treatment may be used to lyse, kill, and remove cells from an ECM or portion thereof. In some embodiments, physical treatment may utilize temperature, force, pressure, and electrical disruption. In some embodiments, temperature methods may be used in a rapid freeze-thaw mechanism. In some embodiments, for example, by freezing a tissue, microscopic ice crystals may form around a plasma membrane and a cell may be lysed. In some embodiments, after lysing one or more cells, a tissue may be further exposed to liquidized chemicals that may degrade and wash out any residual or undesirable components. In some embodiments, temperature methods may conserve a physical structure of an ECM scaffold. In some embodiments, an isolated organ or portion thereof, and a tissue may be decellularized at a suitable temperature. In some embodiments, a suitable temperature may be from about 4° C., 8° C., 10° C., 12° C., 14° C., 16° C., 18° C., 20° C., 22° C., 24° C., 26° C., 28° C., 30° C., 32° C., 34° C., 36° C., 38° C., 40° C., 45° C., 50° C., 55° C., 60° C., or up to about 70° C. In some embodiments, a physical treatment may also include a use of pressure. In some embodiments, pressure decellularization may involve a controlled use of hydrostatic pressure applied to a tissue, isolated organ, or portion thereof. In some embodiments, pressure decellularization may be performed at high temperatures to avoid unmonitored ice crystal formation. In some embodiments, electrical disruption of an isolated organ or portion thereof may be performed. In some embodiments, electrical disruption may be done to lyse cells housed in a tissue or isolated organ or portion thereof. In some embodiments, by exposing a tissue, isolated organ, or portion thereof to electrical pulses, micropores may be formed at a plasma membrane. In some embodiments, one or more cells may die after their homeostatic electrical balance can be ruined through such applied stimulus. In some embodiments, this electrical process can be documented as Non-thermal irreversible electroporation (NTIRE). In some embodiments, sonication may also be used for decellularization or to enhance perfusion decellularization.

### Chemical and Enzymatic Treatments

Also disclosed herein are methods of chemical treatment of an isolated organ or portion thereof to achieve at least partial decellularization. In some embodiments, chemicals and/or salts thereof for use in a chemical treatment may be selected for decellularization depending on: thickness, extracellular matrix composition, intended use of a tissue or isolated organ, or any combination thereof. In some embodiments, for example, enzymes may not be used on a collagenous tissue because they may disrupt connective tissue fibers. In some embodiments, when collagen is not present in a high concentration or needed in a tissue, enzymes may be a viable option for decellularization. In some embodiments, a chemical or salt thereof which may be used to kill and remove cells may be but are not limited to acids, alkaline treatments, ionic detergents, non-ionic detergents, zwitterionic detergents, or any combination thereof. In some embodiments, one or more chemicals may comprise a cellular disruption media. In some embodiments, a cellular disruption media may comprise at least one detergent such as Sodium dodecyl sulfate (SDS), polyethylene glycol (PEG), or Triton X. In some embodiments, detergents may act effectively to lyse a cell membrane and expose content to further degradation. In some embodiments, for example, after SDS lyses a cellular membrane, endonucleases and/or exonucleases may degrade a genetic content, while other components of a cell may be solubilized and washed out of a matrix. In some embodiments, a detergent may be mixed with an alkaline and/or acid treatment due to their ability to degrade nucleic acids and solubilize cytoplasmic inclusions. In some embodiments, one or more cellular disruption media may be used to decellularize an isolated organ or tissue. In some embodiments, a cellular disruption medium may comprise at least one detergent such as SDS, PEG, or Triton X. In some embodiments, a cellular disruption medium may comprise water such that a media may be osmotically incompatible with a cell. In some embodiments, alternatively, a cellular disruption medium may comprise a buffer (e.g., PBS) for osmotic compatibility with a cell. In some embodiments, cellular disruption media also may include enzymes such as, without limitation, one or more collagenases, one or more dispases, one or more DNases, one or more proteases, and any combination thereof. In some embodiments, cellular disruption media also or alternatively may include inhibitors of one or more enzymes (e.g., protease inhibitors, nuclease inhibitors, and/or collagenase inhibitors). In some embodiments, a cellular disruption media may include water such that a media may be osmotically incompatible with a cell. In some embodiments, alternatively, a cellular disruption media may include a buffer (e.g., PBS) for osmotic compatibility with a cell. In some embodiments, cellular disruption media also may include enzymes such as, without limitation, one or more collagenases, one or more dispases, one or more DNases, or a protease such as trypsin. In some embodiments, cellular disruption media also or alternatively may include inhibitors of one or more enzymes (e.g., protease inhibitors, nuclease inhibitors, and/or collagenase inhibitors). In some embodiments, a non-ionic detergent such as Triton X-100 may be utilized. In some embodiments, Triton X-100 may disrupt an interaction between lipids, or between lipids and proteins. In some embodiments, Triton X-100 may not disrupt protein-protein interactions, which may be beneficial to keeping an ECM intact. In some embodiments, EDTA may be utilized. In some embodiments, EDTA may be a chelating agent that binds calcium, which may be a component for proteins to interact with one another. In some embodiments, by making calcium unavailable, EDTA may prevent integral proteins between cells from binding to one another. In some embodiments, EDTA may be used with trypsin, an enzyme that acts as a protease to cleave an already existing bond between integral proteins of neighboring cells within a tissue. In some embodiments, a detergent may be administered from about 10 min, 30 min, 1 hr., 2 hrs., 3 hrs., 4 hrs., 5 hrs., 6 hrs., 7 hrs., 8 hrs., 9 hrs., 10 hrs., 11 hrs., 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some embodiments, depending upon a size and/or weight of an isolated organ or portion thereof a chemical treatment such as a detergent may be contacted with an isolated organ or portion thereof from about 2 hours, 3 hours, 4 hours, 5 hours, 6 hours, 7 hours, 8 hours, 9 hours, 10 hours, 11 hours, 12 hours, 13 hours, 14 hours, 15 hours, to about 20 hours per gram of solid isolated organ or tissue with cellular disruption medium. In some embodiments, including washes, an isolated organ may be perfused for up to about 12 hrs., 13 hrs., 14 hrs., 15 hrs., 16 hrs., 17 hrs., 18 hrs., 19 hrs., 20 hrs., 21 hrs., 22 hrs., 23 hrs., 24 hrs., 25 hrs., 26 hrs., 27 hrs., 28 hrs., 29 hrs., 30 hrs., 31 hrs., 32 hrs., 33 hrs., 34 hrs., 35 hrs., 36 hrs., 37 hrs., 38 hrs., 39 hrs., 40 hrs., 41 hrs., 42 hrs., 43 hrs., 44 hrs., 45 hrs., 46 hrs., 47 hrs., 48 hrs., 49 hrs., 50 hrs., 51 hrs., 52 hrs., 53 hrs., 54 hrs., 55 hrs., 56 hrs., 57 hrs., 58 hrs., 59 hrs., 60 hrs., 70 hrs., 80 hrs., 90 hrs., or up to about 100 hrs. In some embodiments, an isolated organ or portion thereof may be perfused from about 12 hours to about 72 hours per gram of tissue. In some embodiments, perfusion may be adjusted to physiologic conditions including pulsatile flow, rate, pressure, and any combination thereof. In some embodiments, an isolated organ, portion thereof, or tissue may be contacted sequentially with at least two different cellular disruption media. In some embodiments, for example, a first cellular disruption medium may include an anionic detergent such as SDS and a second cellular disruption medium may include an ionic detergent such as Triton X. In some embodiments, following contacting, such as perfusion, with at least one cellular disruption medium, a cannulated isolated organ or tissue may be perfused, for example, with wash solutions or solutions containing one or more enzymes. In some embodiments, alternating a direction of perfusion (e.g., antegrade and retrograde) may help to effectively decellularize an isolated organ, portion thereof, or tissue. For example, by alternating the direction of a perfusion or introducing into an organ or portion thereof, decellularization or recellularization of an organ or portion thereof can be improved as compared to a comparable method absent the alternating of perfusion direction. Improvement of decellularization or recellularization can be at least about 20%, 30%, 40%, 50%, 60%, 80%, 100%, 150%, 200%, 350%, or up to about 500%. In some embodiments, decellularization may decellularize an isolated organ or portion thereof from an inner portion toward an outer portion, resulting in very little damage to an ECM. In some embodiments, a sequential method of decellularization may comprise contacting an isolated organ or portion thereof with a cellular disruption media, such as an SDS detergent, followed by a washing step, followed by an addition of one or more chemicals, followed by contacting with a detergent, and ending with at least one wash step. In some embodiments, a sequential method of decellularization may comprise at least 1, 2, 3, 4, 5, 6, 7, 8, 9, 10, 11, 12, 13, 14, or up to 15 contacting steps with any media or solution disclosed herein. In some embodiments, a buffer disclosed herein may be at a concentration from about 0.1%, 0.5%, 1%, 1.5%, 2%, 2.5%, 3%, 3.5%, 4%, 4.5%, 5%, 10%, 20%, 30%, 40%, 50%, 60%, 70%, 80%, 90%, 95%, or up to about 100%. In some embodiments, an isolated organ, a tissue, or a portion thereof (e.g., liver, lung, kidney, heart, bladder, pancreas, spleen, uterus, or a portion thereof) may be pretreated or flushed with a flushing solution prior to decellularization. In some embodiments, pretreating or flushing an isolated organ, tissue, or a portion thereof with a solution may be used to remove blood and/or reduce blood clot formation. In some embodiments, an isolated organ, a tissue, or a portion thereof may be flushed once, twice, three times, four times, five times, or longer. In some embodiments, flushing occurs over a period of time. In some embodiments, flushing an isolated organ, tissue, or portion thereof may range from a few minutes to days, a few minutes to a few hours, or for a few hours (e.g., for about 5 minutes, 10 minutes, 15 minutes, 20 minutes, 30 minutes, 45 minutes, 1 hour, 2 hours, 3 hours, 6 hours, 10 hours, 24 hours, 48 hours, or longer). In some embodiments, a flushing solution may be a hypertonic solution. In some embodiments, a hypertonic solution may refer to a solution that has a greater concentration of solutes than a concentration of solutes inside of a cell. In some embodiments, a hypertonic solution includes sodium chloride and a second sodium source, such as sodium acetate. In some embodiments, a hypertonic solution includes a source of sodium and a source of chloride. In some embodiments, a ratio of sodium to chloride may be between about 1:0.7 and 1:1. In some embodiments, hypertonic solutions may comprise: NaCl, KCl, NaHCO₃, Na₂SO₄, Na₂HPO₄, MgCl₂, Na acetate, Na lactate, saline, 4× saline, 5× saline, or any combination thereof. In some embodiments, a hypertonic solution can be saline. In some embodiments, a hypertonic solution can be 5× saline. In some embodiments, a hypertonic solution can be about, not less than about, or at most about 0.1% NaCl, 0.2% NaCl, 0.5% NaCl, 0.7% NaCl, 0.8% NaCl, 0.9% NaCl, 1% NaCl, 1.5% NaCl, 2% NaCl 2.5% NaCl, 3% NaCl, 3.5% NaCl, 4% NaCl, 4.5% NaCl, 5% NaCl, 5.5% NaCl, 6% NaCl, 6.5% NaCl, 7% NaCl, 8% NaCl, 9% NaCl, 10% NaCl, 12% NaCl, 15% NaCl, 20% NaCl, 23% NaCl, or 25% NaCl. In some embodiments, a hypertonic solution may include other ingredients, such as KHCO₃, K acetate, K lactate, MgSO₄, or K₂IPO₄. In some embodiments, a saline solution may have a formulation of 10× which may be diluted with water to attain various hypertonic solutions. In some embodiments, a hypertonic solution may range from about 1.01× to about 10× buffered solution. In some embodiments, a buffered solution can be a saline solution. In some embodiments, an isolated organ, a tissue, or any portion thereof may be washed with a hypertonic solution, a hypotonic solution, or both. In some embodiments, an isolated organ, a tissue, or a portion thereof may be disinfected before and/or after decellularization. In some embodiments, disinfection can be performed before decellularization. In some embodiments, disinfection can be performed after decellularization. In some embodiments, disinfection can be performed after an isolated organ, tissue, or portion thereof can be flushed with a flushing solution. In some embodiments, disinfection can be performed without flushing an isolated organ, tissue, or portion thereof with a flushing solution. In some embodiments, disinfection can be performed before an isolated organ, tissue, or portion thereof can be washed with a washing solution. In some embodiments, disinfecting an isolated organ, tissue, or portion thereof may be performed, for example, by placing an isolated organ, tissue, or portion thereof in a disinfection bath with a disinfecting solution. In some embodiments, disinfection may also occur by perfusing or immersing an isolated organ, tissue, or portion thereof with a disinfection solution. In some embodiments, disinfection occurs by submersing an isolated tissue, organ, or portion thereof in a disinfection solution. In some embodiments, a disinfection solution may include an acid, peracid, hydrogen peroxide, peroxide, acetic acid, peracetic acid, and peroxyacetic acid. In some embodiments, a disinfecting solution may comprise one or more of a peracid, hydrogen peroxide, acetic acid, peracetic acid (PAA), saline, SDS, or sodium hydroxide (NaOH). In some embodiments, a solution used for disinfection may comprise an acid (e.g., a peracid), hydrogen peroxide, a chemical compound comprising hydrogen peroxide, a chemical compound comprising a peracid, hydrogen peroxide covalently linked to an organic moiety, saline, and/or a sodium containing solution. In some embodiments, a disinfection solution can comprise saline. In some embodiments, a disinfection solution can comprise sodium. In some embodiments, a disinfection solution can comprise NaCl (e.g., 0.1% NaCl, 0.2% NaCl, 0.5% NaCl, 0.7% NaCl, 0.8% NaCl, 0.9% NaCl, 1% NaCl, 1.5% NaCl, 2% NaCl 2.5% NaCl, 3% NaCl, 3.5% NaCl, 4% NaCl, 4.5% NaCl, 5% NaCl, 5.5% NaCl, 6% NaCl, 6.5% NaCl, 7% NaCl, 8% NaCl, 9% NaCl, 10% NaCl, 12% NaCl, 15% NaCl, 20% NaCl, 23% NaCl, or 25% NaCl). In some embodiments, a disinfection solution can comprise saline and an acid. In some embodiments, a disinfection solution can comprise saline and peracid (e.g., peracetic acid). In some embodiments, a disinfection solution can comprise 0.9% NaCl and 600 ppm peracetic acid. In some embodiments, saline can be 1× saline, 2× saline, 5× saline, 7× saline, 10× saline, 12× saline, 15× saline. In some embodiments, a disinfection solution can comprise saline and a peracid (e.g., peracetic acid). In some embodiments, saline can be 1× saline. In some embodiments, an acid or peracid in a disinfection solution may range from about 25 parts per million (ppm) to about 4000 ppm. In some embodiments, an acid or peracid in a disinfection solution may range from about 500 ppm to about 700 ppm (500 ppm-700 ppm), 600 ppm-650 ppm, 250 ppm-700 ppm, 250 ppm-800 ppm, 550 ppm-1000 ppm, 600 ppm-700 ppm, 550 ppm-2000 ppm, 550 ppm-3000 ppm, 550 ppm-4000 ppm, 1000 ppm-2000 ppm, 2000 ppm-3000 ppm, or 3000 ppm-4000 ppm. In some embodiments, an acid or peracid can be about, at least about, or at most about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm. In some embodiments, an acid or peracid (e.g., peracetic acid) can be about 600 ppm. In some embodiments, an acid or peracid (e.g., peracetic acid) can be about 50 ppm. In some embodiments, a disinfection solution may have a pH of about 4 to about 10 (e.g., pH of 6-7, 5-8, 5-10, 6-8, and 5.5-9). In some embodiments, a disinfecting solution has a pH of between about 5.00 to about 7.50, between about 6.00 to about 8.00, between about 6.10 to about 7.00, between about 4.50 to about 9.00, or between about 6.00 to about 6.50. In some embodiments, a disinfecting solution has a pH of about, at least about, or at most about 4.00, 4.50, 5.00, 5.50, 5.80, 5.90, 6.00, 6.05, 6.10, 6.11, 6.12, 6.13, 6.14, 6.15, 6.16, 6.17, 6.18, 6.19, 6.20, 6.30, 6.40, 6.41, 6.42, 6.43, 6.44, 6.45, 6.50, 6.60, 6.90, 7.00, 7.50, 8.00, 8.50, 9.0, 9.5, 10, 10.5, 11, 11.5, 12, 12.5, 13, 13.5, or 14. In some embodiments, disinfection of an isolated organ, a tissue, or a portion thereof may range from a few minutes, to days, to weeks, to months, or longer. In some embodiments, disinfection can be performed for about, at least about, or at most about 5 minutes, 10 min., 20 min., 30 min., 45 min., 1 hr, 1.5 hrs., 2 hrs., 2.5 hrs., 3 hrs., 3.5 hrs., 4 hrs., 4.5 hrs., 5 hrs., 5.5 hrs., 6 hrs., 6.5 hrs., 7 hrs., 7.5 hrs., 8 hrs., 8.5 hrs., 9 hrs., 9.5 hrs., 10 hrs., 12 hrs., 15 hrs., 18 hrs., 20 hrs., 22 hrs., 24 hrs., 27 hrs., 30 hrs., 34 hrs., 40 hrs., 44 hrs., 48 hrs., or longer. In some embodiments, disinfection can be performed for about, at least about, or at most about a day, two days, three days, four days, five days, six days, seven days, fourteen days, thirty days, or longer. In some embodiments, disinfection can be performed for about, at least about, or at most about a week, two weeks, three weeks, four weeks, five weeks, seven weeks, eight weeks, a month, two months, three months, four months, five months, six months, a year, or longer.

In some embodiments, an isolated organ, tissue, or portion thereof can be decellularized using a decellularization solution comprising an acid (e.g. acetic acid) or a radical generating compound. A radical generating compound can be a compound that is capable of decomposing into a radical under mild conditions. A radical generating compound can include a compound of formula R¹—O—O—R², wherein R¹ and R² can independently be H, C₁-C₆ alkyl, substituted C₁-C₆ alkyl, aryl, substituted aryl, benzyl, substituted benzyl, C(═O)A¹, C(═O)OA², wherein A₁ and A₂ can independently be H, C₁-C₆ alkyl or substituted C₁-C₆ alkyl. In some embodiments, a radical generating compound can be a compound of formula R¹—O—O—R², wherein R¹ is H and R² is C(═O)CH₃. In some cases, a radical generating compound can be a peracid as described herein.

In some embodiments, decellularization of an isolated organ, tissue, or portion thereof can be performed after disinfection. In some embodiments, decellularization of an isolated organ, tissue, or portion thereof can be performed before disinfection. In some embodiments, decellularization of an isolated organ, tissue, or portion thereof can be performed after flushing. In some embodiments, decellularization of an isolated organ, tissue, or portion thereof can be performed before flushing. In some embodiments, decellularization of an isolated organ, tissue, or portion thereof can be performed after flushing and disinfection. In some embodiments, decellularization of an isolated organ, tissue, or portion thereof can be performed before flushing and disinfection. In some embodiments, a solution used for decellularization can comprise a detergent (e.g., sodium dodecyl sulfate (SDS)). In some embodiments, a solution used for decellularization can comprise a detergent and an acid such as a peracid. In some embodiments, a solution used for decellularization can comprise an acid, a peracid, hydrogen peroxide, a chemical compound comprising hydrogen peroxide, a chemical compound comprising a peracid, hydrogen peroxide covalently linked to an organic moiety, saline, and/or a sodium containing solution. In some embodiments, a detergent (e.g., SDS) can be present at an amount of about 0.1%, 0.2%, 0.3%, 0.5%, 0.6%, 0.8%, 0.9%, 1%, 5%, 10%, 15%, 17%, 20%, 25%, 30%, 35%, 40%, 50%, 60%, 65%, 70%, 80%, 90%, or greater. In some embodiments, a detergent can be present at 0.6%. In some embodiments, a detergent can be present at 0.9%. In some embodiments, a detergent can be commercially available. In some embodiments, a solution used for decellularization can comprise a detergent and a peracid (e.g., SDS and peracetic acid (PAA)). In some embodiments, a decellularization solution may have a pH of about 4 to about 10 (e.g., pH of 6-7, 5-8, 5-10, 6-8, and 5.5-9). In some embodiments, a decellularization solution has a pH of between about 5.00 to about 7.50, between about 6.00 to about 8.00, between about 6.10 to about 7.00, between about 4.50 to about 9.00, or between about 6.00 to about 6.50. In some embodiments, a decellularization solution has a pH of about, at least about, or at most about 4.00, 4.50, 5.00, 5.50, 5.80, 5.90, 6.00, 6.05, 6.10, 6.11, 6.12, 6.13, 6.14, 6.15, 6.16, 6.17, 6.18, 6.19, 6.20, 6.30, 6.40, 6.41, 6.42, 6.43, 6.44, 6.45, 6.50, 6.60, 6.90, 7.00, 7.50, 8.00, 8.50, 9.0, 9.5, 10, 10.5, 11, 11.5, 12, 12.5, 13, 13.5, or 14. In some embodiments, decellularization of an isolated organ, a tissue, or a portion thereof may range from a few minutes, to days, to weeks, to months, or longer. In some embodiments, decellularization can be performed for about, at least about, or at most about 5 minutes, 10 min., 20 min., 30 min., 45 min., 1 hr, 1.5 hrs., 2 hrs., 2.5 hrs., 3 hrs., 3.5 hrs., 4 hrs., 4.5 hrs., 5 hrs., 5.5 hrs., 6 hrs., 6.5 hrs., 7 hrs., 7.5 hrs., 8 hrs., 8.5 hrs., 9 hrs., 9.5 hrs., 10 hrs., 12 hrs., 15 hrs., 18 hrs., 20 hrs., 22 hrs., 24 hrs., 27 hrs., 30 hrs., 34 hrs., 40 hrs., 44 hrs., 48 hrs., or longer. In some embodiments, decellularization can be performed for about, at least about, or at most about a day, two days, three days, four days, five days, six days, seven days, fourteen days, thirty days, or longer. In some embodiments, disinfection can be performed for about, at least about, or at most about a week, two weeks, three weeks, four weeks, five weeks, seven weeks, eight weeks, a month, two months, three months, four months, five months, six months, a year, or longer. In some embodiments, a decellularization can comprise: at least one wash step, at least one flushing step, at least one disinfection step, at least one decellularization step. In some embodiments, a peracid solution can be a peroxide solution. In some embodiments, examples of peracid include, but are not limited to, peroxyacetic acid, peroxyoctanoic acid, a sulfoperoxycarboxylic acid, peroxysulfonated oleic acid, peroxyformic acid, peroxyoxalic acid, peroxypropanoic acid, peroxybutanoic acid, peroxypentanoic acid, peroxyhexanoic acid, peroxyadipic acid, perlactic acid, peroxycitric, peroxybenzoic acid, and any combination thereof. In some embodiments, a peracid can be a mixture of hydrogen peroxide and acetic acid. In some embodiments, a peracid can be peracetic acid (PAA). In some embodiments, a peracid (e.g., peracetic acid) can be present at a range of about 0.1% to about 1.0%, of about 0.1% to about 15%, of about 0.1% to about 5.0%, of about 0.1% to about 10%, or of about 0.1% to about 0.5%. In some embodiments, a solution containing a peracid (e.g., peracetic acid) can comprise about 0.1% peracid. In some embodiments, a peracid concentration in a solution (e.g., in a decellularization solution or disinfection solution or another solution used during a decellularization process) can be about, at least about, or at most about 0.1%, 0.3%, 0.5%, 0.8%, 1%, 1.5%, 2%, 2.5%, 3%, 3.5% 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 18%, 20%, 23%, 25%, or more. In some embodiments, an acid or peracid in a solution (e.g., in a decellularization solution or disinfection solution or another solution used during a decellularization process) may range from about 25 parts per million (ppm) to about 4000 ppm. In some embodiments, an acid or peracid in a solution (e.g., in a decellularization solution or disinfection solution or another solution used during a decellularization process) may range from about 500 ppm to about 700 ppm (500 ppm-700 ppm), 600 ppm-650 ppm, 250 ppm-700 ppm, 250 ppm-800 ppm, 550 ppm-1000 ppm, 600 ppm-700 ppm, 550 ppm-2000 ppm, 550 ppm-3000 ppm, 550 ppm-4000 ppm, 1000 ppm-2000 ppm, 2000 ppm-3000 ppm, or 3000 ppm-4000 ppm. In some embodiments, an acid or peracid in a solution (e.g., in a decellularization solution or disinfection solution or another solution used during a decellularization process) can be about, at least about, or at most about 10 ppm, 25 ppm, 50 ppm, 75 ppm, 90 ppm, 100 ppm, 125 ppm, 150 ppm, 175 ppm, 200 ppm, 225 ppm, 250 ppm, 275 ppm, 300 ppm, 325 ppm, 350 ppm, 375 ppm, 400 ppm, 425 ppm, 450 ppm, 475 ppm, 500 ppm, 525 ppm, 550 ppm, 575 ppm, 600 ppm, 625 ppm, 650 ppm, 675 ppm, 700 ppm, 725 ppm, 750 ppm, 775 ppm, 800 ppm, 900 ppm, 1000 ppm, 1200 ppm, 1400 ppm, 1500 ppm, 1700 ppm, 2000 ppm, 2200 ppm, 2500 ppm, 2750 ppm, 3000 ppm, 3200 ppm, 3500 ppm, 3750 ppm, or 4000 ppm. In some embodiments, an acid or peracid (e.g., peracetic acid) can be about 600 ppm. In some embodiments, an acid or peracid (e.g., peracetic acid) can be about 50 ppm. In some embodiments, an addition of an acid or peracid to a solution may increase a flow rate. In some embodiments, an increase in flow rate can be retained even in subsequent steps without an acid or peracid. In some embodiments, an addition of a peracid to a solution during perfusion decellularization may increase a flow rate and a flow rate may be retained (or does not substantially decrease) during subsequent perfusions without a peracid. In some embodiments, a flow rate can be adjusted at a predetermined pressure. In some embodiments, a predetermined pressure can be about, at least about, or at most about 1 mmHg, 2 mmHg, 3 mmHg, 4 mmHg, 5 mmHg, 6 mmHg, 7 mmHg, 8 mmHg, 9 mmHg 10 mmHg, 11 mmHg, 12 mmHg, 13 mmHg, 14 mmHg, 15 mmHg, 16 mmHg, 17 mmHg, 18 mmHg, 19 mmHg, 20 mmHg, 21 mmHg, 22 mmHg, 23 mmHg, 24 mmHg, 25 mmHg, 26 mmHg, 27 mmHg, 28 mmHg, 29 mmHg 30 mmHg, 35 mmHg, 40 mmHg, 45 mmHg, 50 mmHg, 55 mmHg, 60 mmHg, 65 mmHg, 70 mmHg, 75 mmHg, 80 mmHg, 90 mmHg, 100 mmHg, 150 mmHg, 175 mmHg, 200 mmHg, 250 mmHg, 300 mmHg, 350 mmHg, 400 mmHg, 450 mmHg, 500 mmHg, 550 mmHg, 600 mmHg, 700 mmHg, 800 mmHg, 900 mmHg, 1000 mmHg, or more. In some embodiments, a predetermined pressure can be between about 5 mmHg to about 20 mmHg, between about 10 mmHg to about 30 mmHg, between about 5 mmHg to about 50 mmHg, or between about 8 mmHg to about 100 mmHg. In some embodiments, a flow rate can be increased by about, at least about, or at most about 50 ml/min, 100 ml/min, 150 ml/min, 200 ml/min, 250 ml/min, 300 ml/min, 350 ml/min, 400 ml/min, 450 ml/min, 500 ml/min, 550 ml/min, 600 ml/min, 650 ml/min, 700 ml/min, 750 ml/min, 800 ml/min, 900 ml/min, 1000 ml/min, 1500 ml/min, 2000 ml/min, or higher. In some embodiments, a flow rate can be increased above a physiological rate. In some embodiments, a flow rate can be increased above a physiological rate by about 5%, 10%, 15%, 20%, 25%, 30%, 35%, 40%, 45%, 50%, 55%, 60%, 70%, 80%, 90%, 95% or higher. In some embodiments, a flow rate can be increased to about, at least about, or at most about 500 mL/min, 1000 mL/min, 1500 mL/min, 2000 mL/min, 2500 mL/min, or higher. In some embodiments, a flow rate may be measured by chromatography such as by liquid chromatography (LC), gas chromatography (GC), or supercritical fluid chromatography (SFC). In some embodiments, a decellularization process may last about, may be at least about, or at most about 10 days, 8 days, 7 days, 5 days, 4 days, 3 days, 2 days, 1 day or less than 1 day. In some embodiments, a process may last about, be at least about, or at most about 1 hr., 2 hrs., 3 hrs., 4 hrs., 5 hrs., 6 hrs., 7 hrs., 10 hrs., 15 hrs., 18 hrs., 20 hrs., 24 hrs., 30 hrs., 35 hrs., 40 hrs., 45 hrs., 50 hrs., 60 hrs., 70 hrs., 80 hrs., 90 hrs., 100 hrs., or more than about 100 hrs. In some embodiments, a decellularization process includes for example, perfusing or immersing or submerging an isolated organ, tissue, or portion thereof with a detergent solution for about 1 to about 10, about 1 to about 20, about 1 to about 7, about 1 to about 15, about 1 to about 2, about 1 to about 5, or about 1 to about 6 phases or cycles. In some embodiments, a decellularization process includes for example, perfusing or immersing or submerging an isolated organ, tissue, or portion thereof with a detergent solution for a total of about, at least about, or at most about 1 hr., 2 hrs., 3 hrs., 4 hrs., 5 hrs., 6 hrs., 7 hrs., 8 hrs., 9 hrs., 10 hrs., 12 hrs., 15 hrs., 20 hrs., 25 hrs., 30 hrs., 35 hrs., 40 hrs., 45 hrs., 50 hrs., 60 hrs., 70 hrs., 80 hrs., 90 hrs., 100 hrs., or longer. In some embodiments, a decellularization process includes for example, perfusing or immersing or submerging an isolated organ, tissue, or portion thereof with a detergent solution for a total of about 20 hrs. to about 50 hrs., of about 10 hrs. to about 80 hrs., or of about 30 hrs. to about 50 hrs. In some embodiments, a decellularization process includes for example, perfusing or immersing or submerging an isolated organ, tissue, or portion thereof with a peracid (e.g., PAA) solution for about 1 to about 10, about 1 to about 20, about 1 to about 7, about 1 to about 15, about 1 to about 2, about 1 to about 5, about 1 to about 3, about 1 to about 2, or about 1 to about 6 phases or cycles. In some embodiments, a decellularization process includes for example, perfusing or immersing or submerging an isolated organ, tissue, or portion thereof with a peracid solution for a total of about, at least about, or at most about 1 hr., 2 hrs., 3 hrs., 4 hrs., 5 hrs., 6 hrs., 7 hrs., 8 hrs., 9 hrs., 10 hrs., 12 hrs., 15 hrs., 20 hrs., 25 hrs., 30 hrs., 35 hrs., 40 hrs., 45 hrs., 50 hrs., 60 hrs., 70 hrs., 80 hrs., 90 hrs., 100 hrs., or longer. In some embodiments, a decellularization process includes for example, perfusing or immersing or submerging an isolated organ, tissue, or portion thereof with a peracid solution for a total of about 2 hrs. to about 10 hrs., of about 30 hrs. to about 7 hrs., or of about 1 hr. to about 15 hrs. In some embodiments, a detergent solution and a peracid solution overlap. In some embodiments, a detergent solution may comprise a peracid solution. In some embodiments, a decellularization process includes washes with water. In some embodiments, a decellularization process may include: perfusing, immersing or submerging an isolated organ, tissue, or portion thereof with water for a total of about, at least about, or at most about 1 hr., 2 hrs., 3 hrs., 4 hrs., 5 hrs., 6 hrs., 7 hrs., 8 hrs., 9 hrs., 10 hrs., 12 hrs., 15 hrs., 20 hrs., 25 hrs., 30 hrs., 35 hrs., 40 hrs., 45 hrs., 50 hrs., 60 hrs., 70 hrs., 80 hrs., 90 hrs., 100 hrs., or longer. In some embodiments, a decellularization process may include: perfusing or immersing or submerging an isolated organ, tissue, or portion thereof with water for about 1 to about 10, about 1 to about 20, about 1 to about 7, about 1 to about 15, about 1 to about 2, about 1 to about 5, about 1 to about 3, about 1 to about 2, or about 1 to about 6 phases or cycles. In some embodiments, a wash with water may contain PBS (e.g., between about 5% PBS up to about 60% PBS). In some embodiments, water can be specific for an isolated organ, tissue, or portion thereof (e.g., hepatic water for liver decellularization). In some embodiments, a decellularized isolated organ, tissue, or portion thereof can comprise at most about 50% of native cells, 40% of native cells, 35% of native cells, 30% of native cells, 20% of native cells, 10% of native cells, 5% of native cells, 3% of native cells, 2% of native cells, 1% of native cells, or about 0% of native cells. In some embodiments, an at least partially decellularized biological organ or portion thereof is submerged in a solution, and a population of cells are introduced into the vasculature of the at least partially decellularized biological organ or portion thereof, for recellularization. The submerging is performed prior to the introducing.

Also disclosed herein are methods of decellularization of an isolated organ, a tissue, or a portion thereof which may not compromise matrix integrity. In some embodiments, an addition of an acid or peracid to a decellularization process may result in an increase in a flow rate and a decrease in an amount of time required for decellularization. In some embodiments, an addition of an acid or peracid to a decellularization process may result in an increase in a flow rate that is maintained following the removal of the peracid from the solution or use of a non peracid solution. In some embodiments, a decellularization process may result in a purer decellularization (e.g., an increase in cell removal in less time). In some embodiments, a decellularization process may result in a purer decellularization (e.g., reduced residual DNA), as described herein. In some embodiments, an addition of a peracid to a solution or an increase in a flow rate during decellularization does not affect cellular adhesion properties. In some embodiments, an addition of a peracid (e.g., PAA) during decellularization does not affect cell adhesion during recellularization of a decellularized isolated organ, tissue, or portion thereof. In some embodiments, a decellularization using a peracid may result in complete removal of cells. In some embodiments, a decellularization using a peracid may not affect a stiffness or flexibility of an extracellular matrix. In some embodiments, a decellularization can be by perfusion. In some embodiments, a decellularization does not comprise agitation. In some embodiments, a decellularization using a peracid may not compromise integrity of a matrix (e.g., integrity of an ECM). In some embodiments, a decellularization using a peracid may not compromise flexibility, elasticity, or stiffness of a matrix (e.g., ECM). In some embodiments, a decellularization using a peracid may not result in a purer decellularization. In some embodiments, a decellularization using a peracid may not affect a surface property of an isolated organ, tissue, or portion thereof. In some embodiments, a decellularization using a peracid does not affect metabolic activity of an isolated organ, tissue, or portion thereof. In some embodiments, a decellularized isolated organ, tissue, or portion thereof can be introduced into a subject (e.g., a human). In some embodiments, a decellularized isolated organ, tissue, or portion thereof can be introduced into a subject after recellularization. In some embodiments, a decellularized isolated organ, tissue, or portion thereof can be introduced into a subject prior to recellularization. In some embodiments, human cells (e.g., stem cells, progenitor cells, regenerative cells, or isolated organ specific cells) can be introduced into an at least partially decellularized non-human mammal. Also disclosed herein are methods or compositions (e.g., an at least partially decellularized or recellularized isolated organ, tissue, or portion thereof) which may provide for an improved rate of rejection as compared to another method of decellularization or a composition obtained from another method. In some embodiments, a rejection rate can be less than about or at most about 50%, 45%, 40%, 35%, 30%, 25%, 20%, 10%, 9%, 8%, 7%, 6%, 5%, 4%, 3%, 2%, 1%, or 0.5%. In some embodiments, a rejection rate can be decreased if an isolated organ, tissue, or portion thereof can be flushed or pretreated prior to decellularization. In some embodiments, a rejection rate can be decreased if an isolated organ, tissue, or portion thereof can be disinfected prior to decellularization. In some embodiments, a rejection rate can be decreased if an isolated organ, tissue, or portion thereof can be decellularized with a solution comprising a peracid, or an acid, or a peroxide.

In some embodiments, decellularization may comprise decellularizing from about 50%, 60%, 70%, 80%, 85%, 90%, 95%, 96%, 97%, 98%, 99%, or up to about 100% of an isolated organ or portion thereof. In some embodiments, decellularization may comprise decellularizing from about 50%-60%, 50-70%, 60-80%, 60-85%, 70-90%, 70-95%, 75-96%, 75-97%, 80-98%, 80-99%, or up to about 75-100% of an isolated organ or portion thereof.

### Recellularization

Also disclosed herein are methods of at least partially recellularizing an at least partially decellularized isolated organ or portion thereof. In some aspects, the at least partially decellularized isolated organ or portion thereof has been perfusion-decellularized or immersion decellularized. In some embodiments, an isolated organ or tissue may be generated by contacting an at least partially decellularized isolated organ or tissue as described herein with a population of cells. In an aspect, recellularization can comprise the repopulation of an acellular ECM scaffold of tissues or organs with a population of cells. In some cases, recellularization can be merely the seeding of cells into an organ or portion thereof. In another aspect, recellularization comprises seeding and engraftment of cells onto an ECM of an organ or portion thereof. In some cases, recellularization can comprise seeding, engraftment, and reendothelialization. In some aspects, recellularization aims to reconstitute the micro-anatomy of the organ or portion thereof and thereby recreate organ-specific function(s).

In an aspect, perfusion decellularization enables organ scaffolds of organs or portions thereof to retain their native vasculature and so cells are introduced through existing vascular pathways during recellularization. For example, cells can be introduced into a vessel, duct, cavity, lumen, or a combination thereof into a decellularized organ or portion thereof. In another aspect, recellularization can occur via direct cellular injection. For example, a direct cellular injection can be performed into any part of an organ or portion thereof. For instance, cells can be injected into a parenchymal compartment of an organ. In some cases, cells may be directly injected into an organ and also perfused through an existing vascular pathway. In an aspect, directly injecting cells into an organ may introduce a break in a surface of an organ. In another aspect, introducing cells into a parenchyma may require cells to cross a basal membrane of a vessel during recellularization.

In some cases, recellularization comprises culturing cells prior to introducing them into an organ or portion thereof. In some cases, cells can be cultured to expand them prior to an introduction, such as a perfusion. In some cases, cells can be partially differentiated in culture or fully differentiated in culture prior to introduction. In some cases, cells may not be cultured prior to introduction to an organ or portion thereof. In some cases, a solution comprising cells is continuously perfused through the organ or portion thereof. In another aspect, recellularization comprises subjecting an organ or portion thereof to organ-specific shear stress. Organ-specific shear stress may assist in reendothelialization of a part of an organ, such as a vascular tree.

In some embodiments, a recellularization may comprise introduction of undifferentiated cells, partially differentiated cells, fully differentiated cells, partial or fully differentiated stem cells from organoids, dissociation of organoids for partially or fully differentiated organ specific stem cells, or a combination thereof into an at least partially decellularized organ or portion thereof.

In an aspect, recellularization can comprise a repopulation of decellularized ECM scaffolds of tissues or organs with organ-specific cell types, stem cells (e.g. iPSCs or embryonic stem cells (ESCs)), or both types of cells, aiming to reconstitute the micro-anatomy of the organ and thereby recreate the organ-specific function. In an aspect, cells that can be utilized for recellularizing an organ or portion thereof may be adult stem cells, induced pluripotent stem cells (iPSCs), embryonic stem cells (ESCs), umbilical cord blood cells, tissue-derived stem or progenitor cells, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), hepatocytes, endothelial cells, cardiac cells, cardiac progenitor cells, liver cells, liver progenitor cells, kidney cells, kidney progenitor cells, lung cells, lung progenitor cells, or any combination thereof. In some embodiments, regenerative cells may comprise human adult stem cells, human induced pluripotent stem cells (hiPSCs), human embryonic stem cells (hESCs), human umbilical cord blood cells, human tissue-derived stem or progenitor cells, human bone marrow-derived stem or progenitor cells, human blood-derived stem or progenitor cells, organoid, human mesenchymal stem cells (hMSCs), human skeletal muscle-derived cells, human multipotent adult progenitor cells (hMAPCs), human cardiac stem cells (CSCs), human multipotent adult cardiac-derived stem cells, human cardiac fibroblasts, human cardiac microvasculature endothelial cells, human aortic endothelial cells, human bone marrow mononuclear cells (hBM-MNC), human endothelial progenitor cells (hEPC), human hepatocytes, human endothelial cells, human cardiac cells, human cardiac progenitor cells, human liver cells, human liver progenitor cells, human kidney cells, human kidney progenitor cells, human lung cells, human lung progenitor cells, or any combination thereof.

In an aspect, recellularization may comprise regenerative cells. In some embodiments, a population of regenerative cells may be embryonic stem cells, umbilical cord blood cells, tissue-derived stem or progenitor cells, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some embodiments, regenerative cells may be totipotent cells, pluripotent cells, or multipotent cells, and may be uncommitted or committed. In some embodiments, regenerative cells also may be single-lineage cells. In some embodiments, regenerative cells may be undifferentiated cells, partially differentiated cells, or fully differentiated cells. In some embodiments, regenerative cells may comprise embryonic stem cells, progenitor cells, precursor cells, “adult”-derived stem cells including umbilical cord cells and fetal stem cells, or any combination thereof. In some embodiments, regenerative cells that may be used to recellularize an isolated organ or portion thereof disclosed herein may be, without limitation, embryonic stem cells, umbilical cord blood cells, tissue-derived stem or progenitor cells, bone marrow-derived stem or progenitor cells, blood-derived stem or progenitor cells, induced pluripotent stem cells (iPSCs), adipose tissue-derived stem or progenitor cells, mesenchymal stem cells (MSC), skeletal muscle-derived cells, or multipotent adult progenitor cells (MAPC). In some embodiments, additional regenerative cells that may be used include tissue-specific stem cells including cardiac stem cells (CSC), multipotent adult cardiac-derived stem cells, cardiac fibroblasts, cardiac microvasculature endothelial cells, or aortic endothelial cells. In some embodiments, bone marrow-derived stem cells such as bone marrow mononuclear cells (BM-MNC), endothelial or vascular stem or progenitor cells, and peripheral blood-derived stem cells such as endothelial progenitor cells (EPC) also may be used as regenerative cells. In some embodiments, any number of regenerative cells that may be introduced into an at least partially decellularized isolated organ or portion thereof in order to generate an isolated organ or tissue may be dependent on size, weight, or type of an isolated tissue, organ or portion thereof.

In some cases, cells may be exogenous to an isolated organ or portion thereof. In some cases, one or more populations of exogenous cells may be introduced into an isolated organ or portion thereof. In some cases, a first population of exogenous cells may engraft to an organ prior to introduction of a second population of exogenous cells. In some cases, a first population of cells may be functional before a second population of exogenous cells is introduced into an isolated organ or portion thereof. In some cases, “functional” may be determined by comprising at least one of a concentration selected from the group consisting of: i. glucose comprising between about 10 mg/hr and about 1000 mg/hr when the isolated organ or portion thereof comprises a plasma concentration comprising about 5-55 mM, ii. lactate production comprising between about 0.1 g/L and about 2 g/L when the isolated organ or portion thereof comprises a plasma concentration comprising about 0.1-1 mM, iii. oxygen comprising between about 90 mmHg and about 150 mmHg when the isolated organ or portion thereof comprises a plasma concentration comprising about 130 mmHg mM thereof, iv. and any combination thereof. In some cases, a plurality of a first exogenous population of cells can comprise at least about 10 cells, at least about 100 cells, at least about 1000 cells, at least about 10,000 cells, at least about 100,000 cells, at least about 1,000,000 cells, at least about 10,000,000 cells, at least about 100,000,000 cells, at least about 500,000,000 cells, at least about 1,000,000,000, at least about 2,000,000,000 cells, at least about 10,000,000,000 cells, at least about 100,000,000,000 cells, at least about 200,000,000,000 cells, at least about 240,000,000,000 cells. In some cases, a plurality can comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of cells. In some cases, a first exogenous population of cells can comprise endothelial cells. In some cases, endothelial cells can comprise human vein endothelial cells (HUVECs). In some cases, a second exogenous population of cells can comprise embryonic stem cells, iPSCs, organoids, dissociated organoids, umbilical cord blood cells, hepatocytes, tissue-derived stem or progenitor cells, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some cases, at least a portion of a second exogenous population of cells can comprise hepatocytes. In some cases, introducing can be via a cannula. In some cases, any one of glucose consumption, lactate consumption, oxygen consumption, ribose consumption, and glycogen production can be observed in an at least partially recellularized isolated organ or portion thereof as compared to a comparable isolated organ or portion thereof generated by a comparable method absent a functional subset of a first exogenous population of engrafted cells before an introduction of a second exogenous population of cells. In some cases, a portion of a first exogenous population of engrafted cells comprises at least 5% of a first exogenous population of engrafted cells. In some cases, a second exogenous population of cells can be perfused into a liver or portion thereof via a hepatic vein. In some cases, a second exogenous population of cells can be perfused into a liver or portion thereof via a bile duct. In some cases, a second population of cells can comprise hepatocytes.

Also disclosed herein is a method comprising determining a concentration of a factor in an at least partially recellularized isolated organ or portion thereof comprising a first population of cells engrafted thereon; and introducing into an at least partially recellularized isolated organ or portion thereof a second population of cells. In some cases, a first population of cells and a second population of cells are different. In some cases, at least one of a first population of cells or a second population of cells can be exogenous to an at least partially recellularized isolated organ or portion thereof. In some cases, a factor can comprise glucose, lactate, ammonia, oxygen, ribose, or glycogen. In some cases, a first population of cells can comprise endothelial cells. In some cases, a second population of cells can comprise embryonic stem cells, umbilical cord blood cells, hepatocytes, tissue-derived stem or progenitor cells, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof. In some cases, an at least partially recellularized isolated organ or portion thereof can comprise a liver, a kidney, a heart, a lung, a bowel, a skeletal muscle, a bone, a uterus, a bladder, a spleen, a brain, and a pancreas. In some cases, at least one population of exogenous cells can be introduced by perfusing a recellularization solution into an at least partially recellularized isolated organ or portion thereof while an at least partially recellularized isolated organ or portion thereof is at least partially submerged in a liquid that comprises a recellularization solution. In some cases, perfusing can be via a cannula. In some cases, perfusing can be antegrade. In some cases, perfusing can be retrograde. In some cases, perfusion can be via a circulatory system.

Provided herein can also be methods of differentiating a population of cells. In some cases, a population of cells may comprise a stem cell or progenitor cells that can be differentiated. In some instances, cells provided herein can be at least partially differentiated into an organ-specific cell type provided herein. In some instances, cells provided herein can be fully differentiated into an organ-specific cell type provided herein. In some instances, cells provided herein can be differentiated into a liver cell, kidney cell, heart cell, lung cell, pancreatic cell, endothelial cell, blood cell. In some embodiments, cells may be differentiated into hepatocytes. In some embodiments, human embryonic stem cells or induced pluripotent stem cells can be differentiated into hepatocytes, endothelial cells, cholangiocytes, any cells types within the liver, and any combination thereof. In some embodiments, human embryonic stem cells or induced pluripotent stem cells can be differentiated into renal specific cells. In some cases, renal specific cells can comprise podocytes, proximal tubule cells, distal tubule cells, mesangial cells, glomerular endothelium, endothelial cells, any cells types within the kidney, and any combination thereof.

Various methods of differentiation can be utilized. In some embodiments, cells are grown in a media that can support growth. In some embodiments, a media can be changed at regular intervals. In some embodiments, cells may be passaged or split at regular intervals to amplify the number of cells or prevent undirected differentiation. In some embodiments, cells may be grown on a feeder layer of a second type of cell. In some embodiments, cells may be grown in feeder-free conditions. In some embodiments, cells may be grown in spheroids or organoids and then dissociated. In some embodiments, cells may be maintained and cultured using only xeno-free and cGMP compliant conditions. In some embodiments, cells may be grown in a container. In some embodiments, a container may comprise flasks, tissue culture plates, or dishes. In some embodiments, a container may comprise glass or plastic. In some embodiments, a container may be sterile. In some embodiments, one or more media may be used to promote cellular differentiation. In some embodiments, cells can be maintained in culture for at least one, two, three, four, or five passages before commencing seeding into the organ or tissue or differentiation.

In some embodiments, a perfusing solution can comprise a differentiation media, growth media or a maintenance media. In some cases, a perfusion solution can comprise a growth factor, an immune modulating agent, a coagulation modulating agent, an antibiotic, a preservative, a small molecule, a morphogenetic factor, a cytokine, a hormone, an extracellular factor, a nutrient, a carbon source, glucose, a pH, or any combination thereof. In some embodiments, a growth factor may be at least one of: Rantes/CCL5, VEGF, HER2, EGFR, c-met/HGFR, ANGP1 or 2, CCL2, CCR1, CCR2, CCR3, CCR4, CD27, CD40, CD40LG, CD70, CSF1R, CSF2, CX3CL1, CXCL10, CXCL12, CXCL13, CXCL8, CXCR2, CXCR3, CXCR4, DDR2, DLL3, DLL4, ENG, EPHA3, EPHA4, ERBB2, ERBB3, ERBB4, FGF2, FGFR1, FGFR2, FGFR3, FGFR4, FLT1, FLT3, FLT4, GPC3, HGF, IFNB1, IFNG, IGF1R, KDR, KIT, LGALS9, MAPK, MET, NFKB1, NTRK, PDGFRA, PDGFRB, RET, STAT3, TEK, TGFB1, TNFVEGF, DKK-1, FGF, BMP-1, BMP-4, SDF-1, IGF, HGF, a glucocorticoid, an antagonist, a leukotriene antagonist, or any combination thereof. In some cases, a growth factor can be selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 4 (BMP-4), Stromal Cell-Derived Factor 1 (SDF-1), Insulin like Growth Factor (IGF), Hepatocyte Growth Factor (HGF), and any combination thereof.

In some cases, a perfusion solution can comprise a hormone. In some cases, a hormone can comprise erythropoietin (EPO), insulin, secretins, glucagon-like polypeptide 1 (GLP-1), activin, inhibin, adiponectin, adipose-derived hormones, adrenocorticotropic hormone, afamelanotide, agouti signaling peptide, allatostatin, amylin, angiotensin, atrial natriuretic peptide, gastrin, somatotropin, bradykinin, brain-derived neurotrophic factor, calcitonin, cholecystokinin, ciliary neurotrophic factor, corticotropin-releasing hormone, cosyntropin, endothelian, enteroglucagon, fibroblast growth factor 15 (FGF15), GFG15/19, follicle-stimulating hormone, gastrin, gastroinhibitory peptide, ghrelin, glucagon, glucagon-like peptide-1, gonadotropin, gonadotropin-releasing hormone, granulocyte-colony-stimulating factor, growth hormone, growth-hormone-releasing hormone, hepcidin, human chorionic gonadotropin, human placental lactogen, incretin, insulin, insulin analog, insulin aspart, insulin degludec, insulin glargine, insulin lispro, insulin-like growth factor, insulin-like growth factor-1, insulin-like growth factor-2, leptin, liraglutide, luteinizing hormone, melanocortin, melanocyte-stimulating hormone, alpha-melanocyte-stimulating hormone, melanotan II, minigastrin, N-terminal prohormone of brain natriuretic peptide, nerve growth factor, neurotrophin-3, neurotrophin-4, NPH insulin, obestatin, orexin, osteocalcin, pancreatic hormone, parathyroid hormone, peptide hormone, peptide YY, plasma renin activity, pramlintide, preprohormone, prolactin, relaxin, relaxin family peptide hormone, renin, salcatonin, secretin, secretin family peptide hormone, sincalide, teleost leptins, temporin, tesamorelin, thyroid-stimulating hormone, thyrotropin-releasing hormone, urocortin, urocortin II, urocortin III, vasoactive intestinal peptide, and/or vitellogenin.

In some cases, a perfusion solution can comprise an interleukin (IL). In some cases, an interleukin can comprise IL-1, IL-la, IL-2, IL-3, IL-4, IL-5, IL-6, IL-7, IL-8, IL-9, IL-10 IL-11, IL-12; IL-13, IL-14, IL-15, IL-16, IL-17, IL-17A, IL-18, IL-19, IL-20, IL-24, and any combination thereof. In an aspect, a perfusion solution can comprise an interferon. In some cases, an interferon can comprise B4GALT7, IFN gamma, IFN omega, IFN-alpha, IFNA10, IFNA4, IFNA5/IFNaG, IFNA7, IFNB1/IFN-beta, IFNE, IFNZ, IL-28B/IFN-lambda-3, IL-29, IFNA8, LOC100425319, MEMO1, and any combination thereof. In some cases, a perfusion solution can comprise a tumor necrosis factor (TNF). In some cases, a tumor necrosis factor can comprise BLyS/TNFSF138, CD70, LTB, TL1A, TRAIL, CD40L, Fas Ligand, RANKL, TNFSF1, LIGHT, CD30L, EDA-A1, OX-40L, TNFA, TNFSF13, and any combination thereof. In some cases, a perfusion solution can comprise a colony-stimulating factor. In some cases, a colony-stimulation factor such as granulocyte-macrophage colony-stimulating factor (GM-CSF), granulocyte colony-stimulating factor (G-CSF), macrophage colony-stimulating factor (M-CSF) and multipotential colony-stimulating factor (most commonly termed interleukin-3), and any combination thereof.

In some instances, a perfusing solution can comprise a morphogenetic factor. A morphogenetic factor can comprise a bone morphogenetic protein (BMP). In some cases, a BMP can comprise a transforming growth factor beta (TGFbeta) family. Relevant BMPs include but are not limited to: BMP1, BMP2, BMP3, BMP4, BMP5, BMP6, BMP7, BMP8a, BMP8b, BMP10, BMP11, BMP15, and any combination thereof. In some cases, a perfusion solution can comprise inhibitors and activators of BMPs. In some cases, inhibitors and activators of BMPs can comprise noggin, chordin, or a combination thereof.

In some cases, a perfusion solution can comprise an immune modulating agent. In some cases an immune modulating agent can comprise a cytokine, a glucocorticoid, an interleukin-2 receptor (IL2R) antagonist, a leukotriene antagonist, or any combination thereof. In some cases, a perfusion solution can comprise a small molecule. In some cases, a small molecule can be organ-specific. In some cases, a small molecule can comprise PI-3 kinase inhibitor LY 294002, B27, Y27632 (ROCK inhibitor), Recombinant human R-spondin protein Forskolin, SB43—TGFb inhibitor, DAPT-Notch inhibitor, IWP2—Wnt inhibitor, LDN193189—BMP inhibitor and Recombinant human DKK protein.

In some cases, a perfusion solution can comprise anti-angiogenic agents and metabolites. In some cases, anti-angiogenic agents can comprise Bevacizumab, thromobospondin-1 (TSP1), anti-PlGF, anti-VEGF, anti-FGF, ANG-1, ANG-2, ANG-3, ANG-4, TIE-1, TIE-2, c-MET, Notch-1, Notch-2, Notch-3 and Notch-4, Jagged-1, Jagged-2, Dll-1, Dll-3, Dll-4, ephrinA1/EphA2, ephrinB2/EphB4, α5β1, αvβ, αvβ5, MCAM, TGFβ-1, TGFβ-2, TGFβ-3, Sema, Rho-J, CLEC14A, ramucirumab, cetuximab (anti-EGFR antibody), volociximab (anti-integrin-αvβ1 monoclonal antibody), etaricizumab or vitaxin (anti-integrin-αvβ antibody), MEDI3617 or REGN910 (anti-Ang-2 antibody), GAL-F2 (anti-FGF-2 antibody), and any combination thereof. In some instances, a metabolite can comprise tetrahydrobiopterin (BH4), carbonic anhydrase IX (CA-IX), lactate transporters (MCT), glucose, ACAT-1 inhibitor, anti-cholesterol, L-arginine, Indoleamine 2,3 dioxygenase-1 (IDO-1), Epacadostat, glutamine, arginine, fatty acids, and any combination thereof.

In some cases, methods of differentiation may comprise using a growth medium. In some cases, methods of differentiation may comprise E-Cad-FD, a gelatinous protein mixture secreted by Engelbreth-Holm-Swarm (EHS) mouse sarcoma cells-coated tissue plates, matrigel coated plates, Vitronectin-coated tissue plates, fibronectin-coated tissue plates, or cell culture medium. In some embodiments, a cell culture medium can comprise: mTesR1, Essential 8, or Essential 8 Flex.

In an aspect, recellularization can lead to reendothelialization of an organ or portion thereof. Reendothelialization may comprise the growth of endothelial tissue in an organ or portion thereof. In some cases, additional factors, such as growth factors may be introduced to assist in reendothelialization. Factors that can assist in reendothelialization include but are not limited to the following and their family of isomers: vascular endothelial growth factor (VEGF) including all related isomers, fibroblast growth factor (FGF), basic FGF (bFGF), platelet derived growth factor (PDGF), epidermal growth factor (EGF), cytokines, interleukins (such as IL-10), inhibitors, thromobospondin-1, TGF-beta inhibitors and any combination thereof.

Any concentration of a growth factor, small molecule, morphogenetic factor, cytokine, hormone, extracellular factor, metabolite, anti-angiogenic, or nutrient may be added to a cellular culture. A concentration of a growth factor, small molecule, morphogenetic factor, cytokine, hormone, extracellular factor, metabolite, anti-angiogenic, or nutrient may be from about 0.5 ng/ml, 1 ng/ml, 1.5 ng/ml, 2 ng/ml, 10 ng/ml, 20 ng/ml, 30 ng/ml, 40 ng/ml, 50 ng/ml, 100 ng/ml, 200 ng/ml, 300 ng/ml, 500 ng/ml, 600 ng/ml, 800 ng/ml, 900 ng/ml, 1 ug/mL, 1.5 ug/ml, 2 ug/ml, 10 ug/ml, 20 ug/ml, 30 ug/ml, 40 ug/ml, 50 ug/ml, 100 ug/ml, 200 ug/ml, 300 ug/ml, 500 ug/ml, 600 ug/ml, 800 ug/ml, 900 ug/ml, and any combination thereof.

A population of cells can be tested for expression of a marker. In some cases, detection of a marker can be used to determine a lineage of cells. In some cases, markers can assist in identification of a cell. In some cases, markers can be utilized to identify stem cells, progenitor cells, partially differentiated cells, or fully differentiated cells. In some instances, markers can be pluripotency markers such as: TRA-1-60, Oct-4, Nanog, hTERT, Brachy, GSC, HNF4a, Klf4, TAT, TTR, TO, CAR, ApoF, PAX6, c-Myc, CXCR4, FOXA2, SOX17, GATA4, and/or Sox2. In some cases, a cell can be organ-specific and may express such markers. In some cases, a hepatocyte may express: Asialoglycoprotein receptor (ASGPR), AFP, 4α (HNF4α), 1α (HNF 11α), and 1β (HNF1β), glucose-6-phosphatase, albumin, α-1-antitrypsin (AAT), cytokeratin 8 (CK8), cytokeratin 18 (CK18), alcohol dehydrogenase 1, arginase Type I, cytochrome p450 3A4 (CYP3A4), liver-specific organic anion transporter (LST-1). In some cases, several mRNAs which are enriched in fetal hepatocytes can be detected including Fibrinogen alpha chain (FGA), Fibrinogen gamma chain (FGG), Transferrin (TF), and/or Angiotensinogen (AGT).

In some instances, a population of cells can comprise a hepatocyte. In some cases, hepatocytes can express proteins involved in a complement system. In some cases, hepatocytes can maintain sufficiently high serum concentrations, eliminate pathogens, maintain an immune system, and any combination thereof. In some cases, complement proteins can be elevated after inflammatory stimulation. In some cases, hepatocytes can be responsible for production of the most abundant complement C3 in the blood (130 mg/dl). In some cases, hepatocytes can produce other plasma complement components and their soluble regulators. In some cases, plasma complement components and their soluble regulators can comprise (C1r/s, C2, C4, C4bp), alternative (C3, factor B), lectin (mannose-binding lectin (MBL), mannan-binding lectin-associated serine proteases (MASP1-3), MAp19), and terminal (C5, C6, C8, C9) pathways of the complement system as well as soluble regulators (factors I, H, and C1 inhibitor). In some cases, immune cells and endothelial cells can produce these proteins. In some cases, a contribution of immune cells and endothelial cells to plasma levels can be insignificant compared with hepatocytes. In some cases, a transcription of complement genes can be controlled by liver-enriched transcription factors. In some cases, liver-enriched transcription factors can comprise hepatocyte nuclear factors (HNFs), CCAAT/enhancer-binding proteins (C/EBPs), cytokine-activated signals (e.g. NF-κB, STAT3), other transcription factors (e.g. AP-1, estrogen and glucocorticoid receptors), or any combination thereof.

In some instances, cells can be tested for expression of a factor before, during, or after differentiation to determine functionality. In some cases, a factor may be secreted by a cell and may be organ-specific. In some cases, a cell may be a hepatocyte or hepatocyte progenitor and may secrete at least one of: albumin, apolipoprotein F (ApoF), constitutive androstane receptor (CAR), P450, tryptophan dioxygenase (TO), or any combination thereof. In some cases, a hepatocyte may be detected by upregulation of tyrosine aminotransferase (TAT), CYP7A1, or a combination thereof. In some cases, a liver cell can express a cytochrome enzyme. In some cases, a cytochrome enzyme can comprise P450, CYP3A4, CYP3A7, CYP2D6, CYP2C9, CYP2C19, or any combination thereof. In some cases, P450 can play a role in liver metabolism and detoxification.

In some aspects, a recellularized liver or portion thereof, can comprise cells that produce a factor. In some cases production of a factor can be determined at least 2 days, 3 days, 4 days, 5 days, 6 days, 7 days, 8 days, 9 days, 10 days, 14 days, 18 days, 20 days, or 30 days after perfusing a substantially decellularized liver or portion of a liver, or liver matrix or portion thereof with a media comprising cells or a media comprising an additive such as a growth factor. In some cases, an analysis of differentiation may additionally comprise determining activity of a protein or factor. In some cases, determining activity of a protein or factor may be performed by assaying metabolism of the P450 substrates midazolam, tolbutamide, bufuralol, and phenacetin by measuring the formation of their metabolites after 24 hours. In some cases, CYP3A4 metabolizes midazolam to 1′hydroxymidazolam while the metabolism of tolbutamide to 4′hydroxytolbutamide is catalyzed by CYP2C9. In some cases, phenacetin can be converted to acetaminophen by CYP1A2 or CYP2E1. In some cases, bufuralol can be metabolized to 1′hydroxybufuralol by CYP2D6. In some cases, metabolites of all substrates can be detected in hESC-derived hepatocytes, which can demonstrate that P450 isozymes are expressed in these cells. In some cases, other assays for determining functionality of hepatocytes can include: examination of a glycogen storage function in a hESC-derived hepatocyte using periodic-acid Schiff (PAS) staining.

In some cases, to quantify, determine, and/or identify markers, factors, and/or proteins expressed by cells one can use a battery of technologies including but not limited to: western blot, quantitative PCR, microscopy, ELISA, immunohistochemistry, flow cytometry, and any combination thereof.

In some embodiments, a level of a factor secreted into a culture media can approach greater than about 30%, about 40%, about 50%, about 60%, about 70%, about 80%, about 90%, or about 100% as compared to a wildtype organ-specific cell. In an aspect, a level of a factor can be from about 25%, about 50%, about 100%, about 125%, about 150%, about 200%, about 300%, about 400%, about 500%, about 600%, about 700%, about 800%, about 900%, about 1000%, about 2000%, about 4000%, about 6000%, about 8000%, or about 10,000% greater as compared to a wild type organ-specific cell.

In an aspect, cells used for recellularization may receive guidance for their rearrangement and maturation from the ECM and any residual components post decellularization. In some cases, preservation of organ-specific ECM components such as growth factors and various structural proteins can facilitate a recellularization process when utilizing a method as disclosed herein. In an aspect, an organ can be reconstructed by cultivating organ-specific cells in an ECM derived from another organ. In some cases, another organ comprise spleen, hepatocytes, hepatocyte-like cells, or any combination thereof.

In some embodiments, during recellularization, an isolated organ or tissue can be maintained under conditions in which one or more regenerative cells may proliferate, multiply, differentiate, and any combination thereof, in an at least partially decellularized isolated organ or portion thereof. In some embodiments, those conditions may include, without limitation, an appropriate temperature, pressure, electrical activity, mechanical activity, force, an appropriate amount of O₂ and/or CO₂, an appropriate amount of humidity, sterile or near-sterile conditions, and any combination thereof. In some embodiments, during recellularization, an at least partially decellularized isolated organ or tissue and a regenerative cell attached thereto may be maintained in a suitable environment.

In an aspect, at least about 5%, 10%, 20%, 30%, 40%, 50%, 60%, 70%, 80%, 90%, or 100% of an organ can undergo reendothelialization. In an aspect, reendothelialization can be from about 25%, 50%, 100%, 1.25%, 150%, 200%, 300%, 400%, 500%, 600%, 700%, 800%, 900%, 1000%, 2000%, 4000%, 6000%, 8000%, or 10,000% greater when a method provided herein is utilized as compared to a comparable organ or portion thereof that undergoes recellularization absent the method.

In some embodiments, engraftment, seeding, proliferation, survival, differentiation, maturation, metabolic stability, and any combination thereof of cells introduced into an at least partially decellularized isolated organ or portion thereof may be improved by reducing a percent particulate of an isolated organ or portion thereof. In some embodiments, an amount or percent of engraftment, seeding, proliferation, survival, differentiation, and any combination thereof of cells introduced into an at least partially decellularized isolated organ or portion thereof may be improved by about 10%, 20%, 30%, 40%, 50%, 60%, 70%, 80%, 90%, 95%, or up to about 100% as compared to a method absent hyperoxygenation, absent organ submersion, and/or absent a seeding media comprising vein endothelial cells. In some embodiments, regenerative cells as disclosed herein may be allogeneic to an at least partially decellularized isolated organ or portion thereof (e.g., a human decellularized isolated organ or tissue seeded with human regenerative cells), or regenerative cells may be xenogeneic to an at least partially decellularized isolated organ or portion thereof (e.g., a pig decellularized isolated organ or tissue seeded with human regenerative cells). In some cases, an at least partially decellularized isolated organ or portion thereof can be allogeneic to a transplant recipient. In some cases, an at least partially decellularized isolated organ or portion thereof can be autologous to a transplant recipient. In some cases, at least two exogenous populations of cells engrafted thereon can be allogeneic to an extracellular matrix of an at least partially recellularized isolated organ or portion thereof. In some cases, at least two exogenous populations of cells engrafted thereon can be autologous to an extracellular matrix of an at least partially recellularized isolated organ or portion thereof. Allogeneic as used herein refers to cells obtained from the same species as that from which an isolated organ or tissue originated (e.g., self (i.e, autologous) or related or unrelated individuals), while xenogeneic as used herein refers to cells obtained from a species different than that from which an isolated organ or tissue originated.

In an aspect, an at least partially decellularized isolated organ or portion thereof may be recellularized with a number of cells. In some cases, a number of cells can comprise at least about 1×10⁴ cells/g of at least partially decellularized tissue, at least about 1×10⁶ cells/g of at least partially decellularized tissue, at least about 2×10⁶ cells/g of at least partially decellularized tissue, at least about 3×10⁶ cells/g of at least partially decellularized tissue, at least about 4×10⁶ cells/g of at least partially decellularized tissue, at least about 5×10⁶ cells/g of at least partially decellularized tissue, at least about 6×10⁶ cells/g of at least partially decellularized tissue, at least about 6×10⁶ cells/g of at least partially decellularized tissue, at least about 8×10⁶ cells/g of at least partially decellularized tissue, at least about 9×10⁶ cells/g of at least partially decellularized tissue, 1×10⁷ cells/g of at least partially decellularized tissue, at least about 2×10⁷ cells/g of at least partially decellularized tissue, at least about 3×10⁷ cells/g of at least partially decellularized tissue, at least about 4×10⁷ cells/g of at least partially decellularized tissue, at least about 5×10⁷ cells/g of at least partially decellularized tissue, at least about 6×10⁷ cells/g of at least partially decellularized tissue, at least about 6×10⁷ cells/g of at least partially decellularized tissue, at least about 8×10⁷ cells/g of at least partially decellularized tissue, at least about 9×10⁷ cells/g of at least partially decellularized tissue, at least about 1×10⁸ cells/g of at least partially decellularized tissue, at least about 2×10⁸ cells/g of at least partially decellularized tissue, at least about 3×10⁸ cells/g of at least partially decellularized tissue, at least about 4×10⁸ cells/g of at least partially decellularized tissue, at least about 5×10⁸ cells/g of at least partially decellularized tissue, at least about 6×10⁸ cells/g of at least partially decellularized tissue, at least about 6×10⁸ cells/g of at least partially decellularized tissue, at least about 8×10⁸ cells/g of at least partially decellularized tissue, at least about 9×10⁸ cells/g of at least partially decellularized tissue, at least about 1×10⁹ cells/g of at least partially decellularized tissue, at least about 2×10⁹ cells/g of at least partially decellularized tissue, at least about 3×10⁹ cells/g of at least partially decellularized tissue, at least about 4×10⁹ cells/g of at least partially decellularized tissue, at least about 5×10⁹ cells/g of at least partially decellularized tissue, at least about 6×10⁹ cells/g of at least partially decellularized tissue, at least about 6×10⁹ cells/g of at least partially decellularized tissue, at least about 8×10⁹ cells/g of at least partially decellularized tissue, at least about 9×10⁹ cells/g of at least partially decellularized tissue, at least about 1×10¹⁰ cells/g of at least partially decellularized tissue, at least about 2×10¹⁰ cells/g of at least partially decellularized tissue, at least about 3×10¹⁰ cells/g of at least partially decellularized tissue, at least about 4×10¹⁰ cells/g of at least partially decellularized tissue, at least about 5×10¹⁰ cells/g of at least partially decellularized tissue, at least about 6×10¹⁰ cells/g of at least partially decellularized tissue, at least about 6×10¹⁰ cells/g of at least partially decellularized tissue, at least about 8×10¹⁰ cells/g of at least partially decellularized tissue, at least about 9×10¹⁰ cells/g of at least partially decellularized tissue, at least about 1×10¹¹ cells/g of at least partially decellularized tissue, at least about 2×10¹¹ cells/g of at least partially decellularized tissue, at least about 3×10¹¹ cells/g of at least partially decellularized tissue, at least about 4×10¹¹ cells/g of at least partially decellularized tissue, at least about 5×10¹¹ cells/g of at least partially decellularized tissue, at least about 6×10¹¹ cells/g of at least partially decellularized tissue, at least about 6×10¹¹ cells/g of at least partially decellularized tissue, at least about 8×10¹¹ cells/g of at least partially decellularized tissue, at least about 9×10¹¹ cells/g of at least partially decellularized tissue, or at least about 1×10¹² cells/g of at least partially decellularized tissue that can be introduced to an organ or portion thereof. In some cases, an initial introduction of endothelial cells can comprise at or about 1×10⁶ cells/g of at least partially decellularized tissue. In some cases, an initial introduction of hepatocytes can comprise at or about 1×10⁷ cells/g of at least partially decellularized tissue. In some cases, an initial introduction of kidney cells can comprise at or about 1×10⁷ cells/g of at least partially decellularized tissue. In some cases, a range of introduced cells can comprise from about 1×10⁵ cells/g of at least partially decellularized tissue to about 1×10⁸ cells/g of at least partially decellularized tissue. In some cases, a weight of at least partially decellularized tissue can be dry, hydrated or perfused. In some cases, an at least partially decellularized tissue can comprise an at least partially decellularized isolated organ or portion thereof. In some cases, an at least partially decellularized tissue can comprise an at least partially decellularized extracellular matrix.

In some embodiments, different types of cells may have different tendencies regarding a population density such cells will reach. In some embodiments, different isolated tissues, organs, or portions thereof, may be recellularized at different densities. In some embodiments, an at least partially decellularized isolated organ or tissue may be seeded with at least about 1,000 (e.g., at least 10,000, 100,000, 1,000,000, 10,000,000, or 100,000,000) regenerative cells; or may have from at or about 1,000 cells/mg tissue (wet weight, i.e, prior to decellularization) to at or about 10,000,000 cells/mg tissue (wet weight) attached thereto. In some embodiments, regenerative cells may be introduced (“seeded”) into an at least partially decellularized isolated organ or tissue by injection into one or more locations. In some embodiments, at least one type of cell may be introduced into an at least partially decellularized isolated organ or portion thereof. In some embodiments, a cocktail of cells or a population of cells may be injected at multiple positions in an at least partially decellularized isolated organ or tissue or different cell types may be injected into different portions of an at least partially decellularized isolated organ or portion thereof. In some embodiments, in addition to injection, regenerative cells, a population of cells, or a cocktail of cells may be introduced by perfusion into a cannulated decellularized isolated organ or portion thereof. In some embodiments, regenerative cells may be perfused into an at least partially decellularized isolated organ using a perfusion medium, which may then be changed to an expansion and/or differentiation medium to induce growth and/or differentiation of a regenerative cell.

In some embodiments, when a liver or portion thereof is recellularized cells may be perfused into the liver or portion thereof. In some embodiments, cells may be perfused into an isolated liver or portion thereof through the hepatic vein. In some embodiments, cells may be perfused into an isolated liver or portion thereof through the portal vein.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof can be maintained in culture until glucose consumption reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h, about 105 mg/h, about 120 mg/h, about 125 mg/h, about 130 mg/h, about 150 mg/h, about 200 mg/h, about 245 mg/h, about 250 mg/h, about 255 mg/h, about 260 mg/h, about 265 mg/h, about 300 mg/h, about 350 mg/h, about 400 mg/h, about 450 mg/h, about 500 mg/h, about 550 mg/h, or about 600 mg/h, at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until glucose consumption reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, 100 mg/h, about 120 mg/h, about 140 mg/h, about 160 mg/h, about 180 mg/h, about 200 mg/h, about 220 mg/h or about 250 mg/h, at which point hepatocytes can be infused through the hepatic vein and allowed to engraft under pressure-controlled continuous media perfusion.

In some embodiments, an isolated organ or portion thereof is maintained in culture until lactate production reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until lactate production reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h, at which point hepatocytes are infused through the hepatic vein and allowed to engraft under pressure-controlled continuous media perfusion.

In some embodiments, an isolated organ or portion thereof is maintained in culture until oxygen consumption reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until oxygen consumption reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h, at which point hepatocytes are infused through the hepatic vein and allowed to engraft under pressure-controlled continuous media perfusion.

In some embodiments, an isolated organ or portion thereof is maintained in culture until ribose consumption reaches a rate of at least about 5 mM/h, about 10 mM/h, about 15 mM/h, about 20 mM/h, about 25 mM/h, about 30 mM/h, about 35 mM/h, about 40 mM/h, about 45 mM/h, about 50 mM/h, about 55 mM/h, about 60 mM/h, about 65 mM/h, about 70 mM/h, about 75 mM/h, about 80 mM/h, about 85 mM/h, about 90 mM/h, about 95 mM/h, or about 100 mM/h at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until ribose consumption reaches a rate of at least about 5 mM/h, about 10 mM/h, about 15 mM/h, about 20 mM/h, about 25 mM/h, about 30 mM/h, about 35 mM/h, about 40 mM/h, about 45 mM/h, about 50 mM/h, about 55 mM/h, about 60 mM/h, about 65 mM/h, about 70 mM/h, about 75 mM/h, about 80 mM/h, about 85 mM/h, about 90 mM/h, about 95 mM/h, or about 100 mM/h, at which point hepatocytes are infused through the hepatic vein and allowed to engraft under pressure-controlled continuous media perfusion.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof is maintained in culture until glycogen production reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until glycogen production reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h, at which point hepatocytes are infused through the hepatic vein and allowed to engraft under pressure-controlled continuous media perfusion.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof is maintained in culture until ammonia production reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until ammonia production reaches a rate of at least about 5 mg/h, about 10 mg/h, about 15 mg/h, about 20 mg/h, about 25 mg/h, about 30 mg/h, about 35 mg/h, about 40 mg/h, about 45 mg/h, about 50 mg/h, about 55 mg/h, about 60 mg/h, about 65 mg/h, about 70 mg/h, about 75 mg/h, about 80 mg/h, about 85 mg/h, about 90 mg/h, about 95 mg/h, or about 100 mg/h, at which point hepatocytes are infused through the hepatic vein and allowed to engraft under pressure-controlled continuous media perfusion.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof is maintained in culture until von Willebrand Factor (vWF) production reaches a rate of at least about 5 μg/h, about 10 μg/h, about 15 μg/h, about 20 μg/h, about 25 μg/h, about 30 μg/h, about 35 μg/h, about 40 μg/h, about 45 μg/h, about 50 μg/h, about 55 μg/h, about 60 μg/h, about 65 μg/h, about 70 μg/h, about 75 μg/h, about 80 μg/h, about 85 μg/h, about 90 μg/h, about 95 μg/h, or about 100 μg/h at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until von Willebrand Factor (vWF) production reaches a rate of at least about 0.1 μg/h, at least about 0.2 μg/h, at least about 0.3 μg/h, at least about 0.4 μg/h, at least about 0.5 μg/h, at least about 0.6 μg/h, at least about 0.7 μg/h, at least about 0.8 μg/h, at least about 0.9 μg/h, at least about 1 μg/h, at least about 5 μg/h, about 10 μg/h, about 15 μg/h, about 20 μg/h, about 25 μg/h, about 30 μg/h, about 35 μg/h, about 40 μg/h, about 45 μg/h, about 50 μg/h, about 55 μg/h, about 60 μg/h, about 65 μg/h, about 70 μg/h, about 75 μg/h, about 80 μg/h, about 85 μg/h, about 90 μg/h, about 95 μg/h, or about 100 μg/h, at which point hepatocytes are infused through the hepatic vein and allowed to engraft under pressure-controlled continuous media perfusion.

In some cases, an at least partially recellularized isolated organ or portion thereof can comprise a substantially intact vasculature comprising a circulating fluid. In some cases, a circulating fluid can comprise blood or a fraction thereof. In some cases, a circulating fluid can comprise: a concentration of glucose of from about 5 mM to about 100 mM, a concentration of oxygen of from about 100 mmHg to about 500 mmHg, or a combination thereof.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof can be maintained in culture until a glucose concentration of a circulating fluid reaches from about 0.1 g/L, to about 5 g/L, from about 0.5 g/L, to about 5 g/L, from about 1 g/L, to about 5 g/L, from about 2 g/L, to about 5 g/L, from about 3 g/L, to about 5 g/L, from about 4 g/L, to about 5 g/L, from about 0.1 g/L, to about 4 g/L, from about 0.1 g/L, to about 3 g/L, from about 0.1 g/L, to about 2 g/L, from about 0.1 g/L, to about 1 g/L, from about 0.1 g/L, to about 0.5 g/L, or from about 0.5 g/L, to about 4 g/L, at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until a glucose concentration of a circulating fluid reaches from about 0.1 g/L, to about 5 g/L, from about 0.5 g/L, to about 5 g/L, from about 1 g/L, to about 5 g/L, from about 2 g/L, to about 5 g/L, from about 3 g/L, to about 5 g/L, from about 4 g/L, to about 5 g/L, from about 0.1 g/L, to about 4 g/L, from about 0.1 g/L, to about 3 g/L, from about 0.1 g/L, to about 2 g/L, from about 0.1 g/L, to about 1 g/L, from about 0.1 g/L, to about 0.5 g/L, or from about 0.5 g/L, to about 4 g/L, at which point hepatocytes can be infused through the hepatic vein and allowed to engraft under pressure-controlled continuous media perfusion.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof can be maintained in culture until an oxygen concentration of a circulating fluid reaches from about 100 mmHg, to about 500 mmHg, from about 200 mmHg, to about 500 mmHg, from about 300 mmHg, to about 500 mmHg, from about 400 mmHg, to about 500 mmHg, from about 120 mmHg, to about 400 mmHg, from about 200 mmHg, to about 300 mmHg, at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until an oxygen concentration of a circulating fluid reaches from about 100 mmHg, to about 500 mmHg, from about 200 mmHg, to about 500 mmHg, from about 300 mmHg, to about 500 mmHg, from about 400 mmHg, to about 500 mmHg, from about 120 mmHg, to about 400 mmHg, from about 200 mmHg, to about 300 mmHg, at which point hepatocytes can be infused through the hepatic vein and allowed to engraft under pressure-controlled continuous media perfusion.

In some embodiments, an at least partially decellularized isolated organ or portion thereof is seeded with HUVECs through a vein and cultured in a bioreactor with media perfusion through an isolated organ or portion thereof at a constant pressure. In some embodiments, an isolated organ or portion thereof can be maintained in culture until a glucose concentration of a circulating fluid reaches at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 5 g/L, about 10 g/L, about 15 g/L, about 20 g/L, about 25 g/L, about 30 g/L, about 35 g/L, about 40 g/L, about 45 g/L, about 50 g/L, about 55 g/L, about 60 g/L, about 65 g/L, about 70 g/L, about 75 g/L, about 80 g/L, about 85 g/L, about 90 g/L, about 95 g/L, or about 100 g/L, at which point a second type of cell is infused through a vein and allowed to engraft under pressure-controlled continuous media perfusion. In some embodiments, an at least partially decellularized porcine whole liver matrix is seeded with HUVECs through the portal and/or hepatic veins and cultured in a bioreactor with media perfusion through the graft at a constant pressure. In some embodiments, a graft can be maintained in culture until a glucose concentration of a circulating fluid reaches at least about 0.1 g/L, at least about 0.2 g/L, at least about 0.3 g/L, at least about 0.4 g/L, at least about 0.5 g/L, at least about 0.6 g/L, at least about 0.7 g/L, at least about 0.8 g/L, at least about 0.9 g/L, at least about 1 g/L, at least about 5 g/L, about 10 g/L, about 15 g/L, about 20 g/L, about 25 g/L, about 30 g/L, about 35 g/L, about 40 g/L, about 45 g/L, about 50 g/L, about 55 g/L, about 60 g/L, about 65 g/L, about 70 g/L, about 75 g/L, about 80 g/L, about 85 g/L, about 90 g/L, about 95 g/L, or about 100 g/L, at which point hepatocytes can be infused through the hepatic vein and allowed to engraft under pressure-controlled continuous media perfusion.

In some embodiments, seeding human umbilical vein endothelial cells (HUVECs) through the portal and hepatic veins of an isolated liver or portion thereof enables functional reendothelialization of a vascular network and can improve graft flow dynamics during subsequent infusions of hepatocytes when compared to grafts that did not receive HUVECs. In some embodiments, the co-seeded grafts remain functional for albumin production, urea production, ammonia clearance activity, and blood perfusion patency.

In some cases, recellularization comprises introducing a media that is hyperoxygenated. Hyperoxygenation occurs when cells, tissues, and/or organs are exposed to an excess supply of oxygen (O₂) or higher than normal partial pressure of oxygen. Maintaining hyperoxic conditions may improve recellularization as compared to methods absent hyperoxygenation or media that is hyperoxygenated. In some cases, a media can comprise at least about 20%, 21%, 22%, 22.5%, 23%, 24%, 25%, 26%, 27%, 28%, 29%, 30%, 31%, 32%, 33%, 34%, 35%, 36%, 37%, 38%, 39%, 40%, 45%, 50%, 55%, 60%, 65%, 70%, 75%, 80%, 85%, 90%, 95%, 100%, 150%, 200%, 250%, 300%, 350%, or up to about 400% pO₂ 140 mmHg as measured by a Jenway® Model 970 Dissolved Oxygen Meter and Electrode. Maintaining hyperoxic conditions may improve recellularization from about 25%, 50%, 100%, 125%, 150%, 200%, 300%, 400%, 500%, 600%, 700%, 800%, 900%, 1000%, 2000%, 4000%, 6000%, 8000%, or 10,000% as compared to methods absent hyperoxygenation or media that is hyperoxygenated. In some instances, improved recellularization may comprise improved seeding, improve reendothelialization, improved differentiation, improved persistence, improved engraftment, and/or improved functionality of cells and/or organs.

In some embodiments, recellularization may be improved by at least partially submerging an at least partially decellularized organ or portion thereof in a solution, before, during, and/or after introducing a population of cells into the organ or portion thereof. In an aspect, submerging can be performed prior to the introducing of the population of cells. In an aspect, submerging can be performed prior to and during the introducing of the population of cells. In an aspect, submerging an organ or portion thereof comprises having at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% of the at least partially decellularized biological organ or portion thereof submerged in solution.

In some cases, by at least partially submerging an at least partially decellularized organ or portion thereof in a solution before, during, or after recellularization may confer 25%, 50%, 100%, 1.25%, 150%, 200%, 300%, 400%, 500%, 600%, 700%, 800%, 900%, 1000%, 2000%, 4000%, 6000%, 8000%, 10,000%, 20,000%, 40,000%, 60,000%, 80,000%, or 100,000% greater recellularization as compared to methods absent submergement. In some instances, improved recellularization may comprise improved seeding, improve reendothelialization, improved differentiation, improved persistence, improved engraftment, and/or improved functionality of cells and/or organs. In an aspect, submerging results in a higher percentage of cells engrafting in a distal area of the at least partially decellularized biological organ or portion thereof as compared to a comparable method absent the submerging. In an aspect, submerging prevents dehydration of the at least partially decellularized biological organ or portion thereof. In an aspect, submerging results in a lower dehydration of the at least partially decellularized biological organ or portion thereof as compared to a comparable method absent the submerging. In an aspect, submerging results in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower dehydration of the at least partially decellularized biological organ or portion thereof as compared to a comparable method absent the submerging. In some cases, submerging results in at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower dehydration during the introducing as compared to a comparable method absent the submerging. Additionally, in some instances, submerging results in improved patency of the vasculature, as compared to the patency of a comparable vasculature obtained from a comparable method absent the submerging. In an aspect, a comparable method that is absent submergement comprises suspending the at least partially decellularized biological organ or portion thereof in air instead of submerging the at least partially decellularized biological organ or portion thereof in the solution.

### Evaluation of Organs and Portions Thereof

Also disclosed herein In some embodiments, are methods to evaluate the function of an at least partially recellularized isolated organ or portion thereof. In some cases, a blood patency of an isolated organ or portion thereof can be measured. In some cases, a measurement of a blood patency of an isolated organ or portion thereof can comprise measurement of a vasculature of an isolated organ or portion thereof. In some cases, a measurement of a blood patency of a vasculature of an isolated organ or portion thereof can comprise measurement using a blood patency detection device. In some cases, a blood patency detection device can comprise a perivascular flow module. In some cases, a perivascular flow module can comprise an ultrasonic perivascular flow module. In some cases, a blood patency can comprise at least about 10 mL/min, at least about 20 mL/min, at least about 30 mL/min, at least about 40 mL/min, at least about 50 mL/min, at least about 60 mL/min, at least about 70 mL/min, at least about 80 mL/min, at least about 90 mL/min, at least about 100 mL/min, at least about 110 mL/min, at least about 120 mL/min, at least about 130 mL/min, at least about 140 mL/min, at least about 150 mL/min, at least about 160 mL/min, at least about 170 mL/min, at least about 180 mL/min, at least about 190 mL/min, at least about 200 mL/min, at least about 210 mL/min, at least about 220 mL/min, at least about 230 mL/min, at least about 240 mL/min, at least about 250 mL/min, at least about 260 mL/min, at least about 270 mL/min, at least about 280 mL/min, at least about 290 mL/min, at least about 300 mL/min, at least about 310 mL/min, at least about 320 mL/min, at least about 330 mL/min, at least about 340 mL/min, at least about 350 mL/min, at least about 360 mL/min, at least about 370 mL/min, at least about 380 mL/min, at least about 390 mL/min, or at least about 400 mL/min. In some cases, a measurement of blood patency can comprise a pressure of about 1 mmHg, about 2 mmHg, about 3 mmHg, about 4 mmHg, about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, or about 20 mmHg.

In some cases, a fluid passing through an at least partially recelullarized organ or portion thereof can be analyzed. In some cases, a fluid passing through an at least partially recelullarized organ or portion thereof can be analyzed by an analyzer such as a flow meter. In some cases, a rate of clearance of a substance from a fluid can be measured. In some cases, a fluid can comprise blood. In some cases, a fluid can comprise plasma. In some cases, an ammonia clearance rate of an at least partially decellularized isolated organ or portion thereof can be measured. Various means of measuring fluid can be utilized and are known to the skilled artisan. In some aspects a flow meter can be utilized. A flow meter can comprise an ultrasound, probe, microscopy, transducer, beam, doppler, catheter, sensor, computer, and any combination thereof. In an aspect, a flow meter is a doppler ultrasound. In an aspect, a flow meter is a flow probe. In some cases, an at least partially recellularized isolated organ or portion thereof can clear ammonia at a rate of at least about 0.01 mmol/hr, at least about 0.02 mmol/hr, at least about 0.03 mmol/hr, at least about 0.04 mmol/hr, at least about 0.05 mmol/hr, at least about 0.06 mmol/hr, at least about 0.07 mmol/hr, at least about 0.08 mmol/hr, at least about 0.09 mmol/hr, at least about 0.1 mmol/hr, at least about 0.2 mmol/hr, at least about 0.3 mmol/hr, at least about 0.4 mmol/hr, at least about 0.5 mmol/hr, at least about 0.6 mmol/hr, at least about 0.7 mmol/hr, at least about 0.8 mmol/hr, at least about 0.9 mmol/hr, at least about 1 mmol/hr, at least about 5 mmol/hr, at least about 10 mmol/hr, at least about 20 mmol/hr, at least about 30 mmol/hr, at least about 40 mmol/hr, at least about 50 mmol/hr, at least about 60 mmol/hr, at least about 70 mmol/hr, at least about 80 mmol/hr, at least about 90 mmol/hr, or at least about 100 mmol/hr, from a circulating fluid as measured by an ultrasonic perivascular flow module. In some cases, an at least partially recellularized isolated organ or portion thereof comprises an at least partially intact vasculature comprising a circulating fluid, wherein an at least partially recellularized isolated organ or portion thereof can maintain an ammonia concentration of a circulating fluid at a level of less than about 0.4 mM in a time period of about 24 hours, as measured by an analyzer device. In some cases, an ammonia clearance rate can be measured by an analyzer. In some cases, an analyzer can comprise an ammonia analyzer. In some cases, an analyzer can comprise a cell culture analyzer. In some cases, an analyzer can comprise an automated analyzer. In some cases, an analyzer can measure a member selected from the group consisting of: glucose, glutamine, ammonium, ammonia, pO₂, sodium, ionized calcium, lactate, glutamate, pH, pCO₂, potassium, cell density, cell viability, cell diameter, osmolality, IgG, PO₄, and any combination thereof.

In some cases, cellular engraftment can be measured by hematoxylin and eosin (H&E) staining. In some cases, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, at least about 150%, at least about 200%, at least about 250%, at least about 300%, at least about 350%, at least about 400%, at least about 450%, at least about 500%, or at least about 1000% more cellular engraftment can be observed as compared to an otherwise comparable isolated organ or portion thereof generated by engrafting a second exogenous population of cells onto a decellularized organ or portion thereof that lacks a functional subset of a first exogenous population of cells.

### Bioengineered Organs and Portions Thereof

Provided herein is also a bioengineered organ. In an aspect, the organ is a bioengineered liver, a bioengineered kidney, or a portion of any of these. The bioengineered organ can be a human organ or a non-human organ. In an embodiment, a bioengineered organ is non-human and from a pig. In another embodiment, a bioengineered organ is from a human cadaver. Bioengineered organs provided herein can be seeded with any cell types disclosure herein. In an embodiment, a cell that is seeded is a primary cell. In some cases, bioengineered organs and portions thereof can be seeded with native cells from human cadaver donors or non-human organs, such as pigs. These bioengineered organs and portions thereof can be seeded with iPSC cells to generate an iPSC-derived bioengineered organ. In an embodiment the organ is a liver. In another embodiment the organ is a kidney.

In some cases, a bioengineered organ is a liver or portion thereof. A bioengineered liver (BEL) can be seeded with cells described herein. In an embodiment, the BEL, can be seeded with endothelial cells, hepatocytes, cholangiocytes, stem cells, or any combination thereof. In an embodiment, the BEL can be seeded with hepatocytes.

In some cases, a BEL can be utilized for the treatment or reduction of acute liver failure. Acute liver failure can be caused by a variety of conditions and diseases. In an embodiment, a BEL can be utilized to detoxify a subject experiencing acute liver failure as either a bridge to transplant or to allow the subject to recover. After treatment, the BEL can be disconnected and/or discarded. In an embodiment, the BEL is sufficient to reduce or eliminate excess ammonia, decrease ICP, provide liver function acutely, or any combination thereof.

In some cases, a bioengineered organ is a kidney or portion thereof. A bioengineered kidney (BEK) can be seeded with any of the cells provided herein. In an embodiment, a BEK is seeded with podocytes, endothelial cells, mesangial cells, stem cells, or any combination thereof. A BEK can be utilized for the treatment or reduction of kidney disease, including kidney failure or end-stage renal disease (ESRD). In an embodiment, a BEK disclosed herein can be used as a bridge to transplant. A BEK provided herein can offer significant advantages compared to current technologies including: 1) a full thickness, biological, kidney-derived matrix material; 2) a vascular supply with appropriately sized vessels; 3) at least partially seeded with human cells; 4) the ability to grow and mature with a treated subject; and 5) a one-time surgical treatment that would significantly reduce or eliminate the need for dialysis, improving the quality of life for ESRD patients. In an embodiment, the BEK is seeded with all human cells. In some cases, a subject that may be eligible for treatment with a BEK provided herein has a glomerular filtration rate (eGFR) that has declined to 10 mL/min/1.73 m², or approximately 90% of lost kidney function.

In an embodiment, a BEK provided herein can provide a greater than: 5%, 10%, 20%, 30%, 40%, 50%, 60%, 70%, 80%, 90%, or 100% reduction in dialysis. In an embodiment, a BEK provided herein can achieve at least about 5%, 10%, 15%, 20%, 25%, 30%, 40%, 50%, 60%, 70%, 80%, 90%, or 100% overall renal function as compared to a non-bioengineered kidney.

In an aspect, any of the bioengineered organs provided herein can extend the time to transplant in a subject by at least about 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, or up to about 5 years. In an aspect, a subject that is treated with a subject bioengineered organ or portion thereof experiences a reduction in excess toxicity (excess ammonia, ICP, or both) by at least about 1 fold, 2 fold, 3 fold, 5 fold, 8 fold, 10 fold, 20 fold, 30 fold, 40 fold, 50 fold, 60 fold, 70 fold, 80 fold, 90 fold, 100 fold, 150 fold, 200 fold, 300 fold, 400 fold, or 500 fold as compared to an otherwise comparable subject lacking the treatment with the bioengineered organ.

In some cases, any of the bioengineered organs provided herein can be recellularized or reseeded with cells disclosed herein. In an embodiment, the bioengineered organ can be recellularized at least or at most: 10%, 20%, 30%, 40%, 50%, 60%, 70%, 80%, 90%, or 100% as compared to a non-bioengineered otherwise comparable organ. Recellularization can be determined using various in vitro assays such as those provided herein, including but not limited to histology, IHC, analyzing organ function, microscopy, or angiography. Potentially relevant markers to analyze include CD31, Trichrome, collagen I, vimentin, e-cadherin, Ki67, and combinations thereof.

### Uses of Organs and Portions Thereof

Also disclosed herein are methods and uses of decellularized and recellularized isolated organs or portions thereof in a variety of applications. In some embodiments, isolated organs or portions thereof may be implanted into a subject. In some embodiments, a composition as described herein, such as an isolated organ or portion thereof, may be transplanted into a subject that has a disease. In some cases, a subject can be a patient. In some cases, a subject can have liver disease, hypertension, diabetes, heart failure, lung disease, kidney disease, or any combination thereof. In some cases, liver disease can comprise cirrhosis, nonalcoholic steatohepatitis, hepatocellular carcinoma, metabolic disease, or any combination thereof. In some cases, treatment can further comprise administering an immunosuppressive condition to a subject. In some cases, a subject can exhibit an intercranial pressure of less than about 50 mmHg, about 30 mmHg, about 25 mmHg, about 20 mmHg, about 18 mmHg, about 15 mmHg, or about 10 mmHg for a duration of at least about 0.5, about 1, about 2, about 3, about 4, about 5, about 6, about 7, about 8, about 9, or about 10 hours during a time period of at least 24 hours after implanting of an at least partially recellularized organ as disclosed herein. In some embodiments, relevant diseases that may require isolated organ transplantation include but are not limited to: isolated organ failure, cardiomyopathy, cirrhosis, chronic obstructive pulmonary disease, pulmonary edema, biliary atresia, emphysema and pulmonary hypertension, coronary heart disease, valvular heart disease, congenital heart disease, coronary artery disease, pancreatitis, cystic fibrosis, diabetes, hepatitis, hypertension, idiopathic pulmonary fibrosis, polycystic kidneys, short gut syndrome, injury, birth defects, genetic diseases, autoimmune disease, and any combination thereof. In some embodiments, implants may be used to replace or augment existing tissue, for example, to treat a subject with a kidney disorder by replacing a dysfunctional kidney of a subject with an exogenous or engineered kidney. In some embodiments, a subject may be monitored after implantation of an exogenous kidney, for amelioration of a kidney disorder. In some embodiments, a decellularized isolated organ or portion thereof disclosed herein may be utilized for implantation into a subject. In some embodiments, a composition disclosed herein, such as a solid isolated organ or portion thereof may have from about 1% to about 100% of its native function after decellularization. In some embodiments, a composition disclosed herein, such as a solid isolated organ or portion thereof may have from about 5%, 10%, 20%, 30%, 40%, 50%, 60%, 70%, 80%, 90%, 95%, or up to about 100% of its native function after decellularization. In some embodiments, particular isolated organs or portions thereof may be suitable for transplantation when they function below that of their native counterpart. In some embodiments, for example, a liver and a kidney may need approximately from about 20% of a total organ function to save a person from liver failure or remove them from dialysis. In some embodiments, a liver and kidney may need approximately from about 20-30%, about 30-40%, about 20-50%, about 20-60%, or about 40-60% of a total organ function to be suitable for transplantation. In some embodiments, an isolated organ may function equal to a native counterpart. In some embodiments, for example, a heart may be more complicated, in that, it may need from about 80%, about 85%, about 90%, about 95%, about 96%, about 97%, about 98%, about 99%, or up to about 100% function at a time of transplantation. In some embodiments, an isolated organ such as a pancreas or lung may function and be transplantable from about 15%, about 20%, about 30%, about 40%, or about 50% of native function. In some embodiments, a pancreas may function and be transplantable from about 10% or more function. In some embodiments, an isolated organ disclosed herein may be used as an accessory organ to help with native function. In some embodiments, a lifespan of a subject may be extended after transplantation of a composition, such as an isolated organ or portion thereof disclosed herein. In some embodiments, for example, a lifespan of a subject may be extended from about 1 month, about 2 months, about 3 months, about 4 months, about 5 months, about 6 months, about 7 months, about 8 months, about 9 months, about 10 months, about 11 months, about 1 year, about 2 years, about 3 years, about 4 years, about 5 years, about 6 years, about 7 years, about 8 years, about 9 years, about 10 years, about 15 years, about 20 years, about 30 years, about 40 years, about 50 years, about 60 years, about 70 years, about 80 years, about 90 years, or up to about 100 years after transplantation. In some embodiments, transplantation of a composition, such as an isolated organ or portion thereof disclosed herein, may reduce a need for a secondary treatment in a subject. In some embodiments, secondary treatments may refer to dialysis, pacemakers, respirators, and combinations thereof. In some embodiments, at least partially decellularized isolated organs or portions thereof or at least partially recellularized isolated organs or portions thereof may be used in vitro to screen a wide variety of compounds, for effectiveness and cytotoxicity of pharmaceutical agents, chemical agents, growth factors, or regulatory factors. In some embodiments, a culture may be maintained in vitro and exposed to a compound to be tested. In some embodiments, an activity of a cytotoxic compound may be measured by its ability to damage or kill cells in culture. In some embodiments, this may readily be assessed by vital staining techniques. In some embodiments, an effect of growth or regulatory factors may be assessed by analyzing a cellular content of a matrix, e.g., by total cell counts, and differential cell counts. In some embodiments, this may be accomplished using standard cytological and/or histological techniques including a use of immunocytochemical techniques employing antibodies that define type-specific cellular antigens. In some embodiments, an effect of various drugs on normal cells cultured in a reconstructed artificial isolated organ may be assessed. In some embodiments, decellularized and recellularized isolated organs or portions thereof which may be used in vitro to filter aqueous solutions, for example, a reconstructed artificial kidney may be used to filter blood. In some embodiments, using a reconstructed kidney provides a system with morphological features that resemble an in vivo kidney product, which in some embodiments, may be suitable for hemodialysis, or for hemofiltration to remove water and low molecular weight solutes from blood. In some embodiments, an artificial kidney may be maintained in vitro and exposed to blood which may be infused into a luminal side of an artificial kidney. In some embodiments, a processed aqueous solution may be collected from an abluminal side of an engineered kidney. In some embodiments, an efficiency of filtration may be assessed by measuring an ion, or metabolic waste content of a filtered and unfiltered blood. In some embodiments, decellularized and recellularized isolated organs or portions thereof may be used as a vehicle for introducing genes or gene products in vivo to assist or improve a result of a transplantation, or for use in gene therapies. In some embodiments, cultured cells, such as endothelial cells, may be engineered to express gene products. In some embodiments, cells may be engineered to express gene products transiently or under inducible control, or as a chimeric fusion protein anchored to an endothelial cell, for example, a chimeric molecule composed of an intracellular and/or transmembrane domain of a receptor or receptor-like molecule, fused to a gene product as an extracellular domain. In some embodiments, one or more endothelial cells may be genetically engineered to express a gene for which a patient can be deficient, or which would exert a therapeutic effect. In some embodiments, a gene of interest engineered into an endothelial cell or parenchyma cell may be related to a disease being treated. In some embodiments, for example for a kidney disorder, an endothelial or cultured kidney cell may be engineered to express gene products that may ameliorate a kidney disorder. In some embodiments, at least two populations of cells may be introduced into an at least partially decellularized isolated organ or portion thereof. In some embodiments, isolated organs that may be engineered include, but are not limited to, heart, kidney, liver, pancreas, spleen, bladder, uterus, ureter, urethra, skeletal muscle, small and large bowel, esophagus, stomach, brain, spinal cord and bone.

In some embodiments, a subject may require chronic dialysis or a kidney transplant for survival. In some embodiments, kidney transplantation can be the only potential curative option. In some embodiments, kidney transplantation can be associated with a 5-year survivability rate >90% compared to a <40% 5 year survivability rate for dialysis. In some embodiments, kidney transplantation can be associated with lower morbidity, and better quality of life than dialysis treatment. In some embodiments, kidney transplantation can be limited by an ongoing and severe shortage of donor kidney grafts for transplantation. In some embodiments, a shortage of donor kidneys can be caused by a limited number of suitable donor grafts. In some embodiments, living donation programs and extended criteria for deceased donor transplantation can not compensate to provide enough kidney grafts available for the increasing number of patients suffering from kidney failure.

Other embodiments and uses will be apparent to those skilled in the art from consideration of the specification and practice of the disclosed herein.

In some embodiments, decellularized isolated organs or portions thereof may be utilized as acellular compositions. Disclosed herein may be an acellular or substantially acellular isolated organ or portion thereof.

### Kits

Disclosed herein may be kits comprising at least partially decellularized mammalian isolated organs or portions thereof. Provided herein can also be at recellularized organs or portions thereof. In some embodiments, a kit may include an at least partially decellularized isolated organ or portion thereof and instructions for use thereof. In some embodiments, a kit can comprise a sterile container which may contain the at least partially decellularized isolated organ or portion thereof; such containers may be boxes, ampules, bottles, vials, tubes, bags, pouches, blister-packs, or other suitable container forms known in the art. Such containers may be made of plastic, glass, laminated paper, metal foil, or other materials suitable for holding isolated organs or portions thereof. In some embodiments, a delivery vehicle composition may be dehydrated, stored and then reconstituted such that a substantial portion of an internal content can be retained. In some embodiments, the kit can comprise a compressed packaging containing an at least partially decellularized isolated organ or portion thereof. For example, a sterile pouch containing an isolated organ or portion thereof may be degassed or otherwise compressed into a sheet. In some cases, a kit may also provide cells for recellularization of a decellularized organ or portion thereof.

### Systems Comprising Organs and Portions Thereof

In some embodiments, a system for generating an isolated organ or portion thereof or tissue may be controlled by a computer-readable storage medium in combination with a programmable processor (e.g., a computer-readable storage medium as used herein has instructions stored thereon for causing a programmable processor to perform particular steps). In some embodiments, for example, such a storage medium, in combination with a programmable processor, may receive and process information from one or more sensors. In some embodiments, such a storage medium in conjunction with a programmable processor also may transmit information and instructions back to a bioreactor and/or an isolated organ or tissue. In some embodiments, an isolated organ or tissue undergoing recellularization may be monitored for biological activity. In some embodiments, biological activity may be that of an isolated organ or portion thereof or tissue itself such as for cardiac tissue, electrical activity, mechanical activity, mechanical pressure, contractility, and/or wall stress of an isolated organ or tissue. In some embodiments, a biological activity of cells attached or engrafted on to an isolated organ or portion thereof or tissue may be monitored, for example, for ion transport/exchange activity, cell division, and/or cell viability. In some embodiments, it may be useful to simulate an active load on an isolated organ or portion thereof during recellularization. In some embodiments, a computer-readable storage medium, in combination with a programmable processor, may be used to coordinate components necessary to monitor and maintain an active load on an isolated organ or tissue. In some embodiments, a weight of an isolated organ or portion thereof or tissue may be entered into a computer-readable storage medium as described herein, which, in combination with a programmable processor, may calculate exposure times and perfusion pressures for that particular organ or tissue. In some embodiments, such a storage medium may record preload and afterload (the pressure before and after perfusion, respectively) and a rate of flow. In some embodiments, for example, a computer-readable storage medium in combination with a programmable processor may adjust a perfusion pressure, a direction of perfusion, and/or a type of perfusion solution via one or more pumps and/or valve controls.

In some cases, a perfusion solution can comprise a decellularization solution. In some cases, a perfusion solution can comprise a recellularization solution. In some cases, a perfusion solution can comprise a dissolved oxygen concentration. In some cases, a dissolved oxygen concentration can comprise at least about 22.5% pO₂ 140 mmHg. In some cases, a dissolved oxygen concentration can be measured by a Jenway Model 970 dissolved oxygen meter and electrode. In some cases, an at least partially recellularized isolated organ or portion thereof can be operatively coupled to a pump. In some cases, a pump can comprise a peristaltic pump or a vacuum pump. In some cases, a system can further comprise a cannula, a perfusion apparatus, a holding container, a tubing, a pump, a sensor, a thermometer, an electrode, a valve, a balloon, a pacemaker, a thermostat, a user interface, or any combination thereof. In some cases, a sensor can comprise a glucose sensor, an ammonia sensor, an oxygen sensor, a fluid sensor, a temperature sensor, a pressure sensor, or any combination thereof.

In an aspect, a bioreactor may be utilized as part of a system provided herein. A bioreactor may need to supply an organ or portion thereof with physical stimulation, electrical stimulation, chemical stimulation, or a combination of these, depending on the situation. For example, a heart or heart tissue may need to be mechanically stretched and/or electrically stimulated. Mechanically stretching tissue facilitates cell alignment, elongation, and expression of connexin-43, a cardiac marker. Electrical stimulation can also be used in tandem with mechanical stretching. The lung may have particular considerations when used in a bioreactor. As the lung is inflated and deflated, the ECM should retain appropriate mechanical compliance. In order to accomplish this, the common method used in a bioreactor is to suspend the lung scaffold in a container of media and to connect the lungs to a ventilator that matches the volume and respiratory rate of the mammal to which it will be transplanted or utilized in. For example, in a lung transplanted from a human, with the bioreactor design, it is promising to recellularize a lung to such an extent that improvement in oxygen exchange can be demonstrated with the recellularized lung at 7 days. In another aspect, a kidney or portion thereof does not necessitate mechanical stimulation. In some cases, but benefits from chemical stimulation. For example, when kidneys are cultured with convert growth factor b1 and trans-retinoic acid, renal proximal tubule cells grow as a monolayer and produce lumens with polarized epithelial layers, microvilli, and tight junction complexes; this may not occur without these factors.

In an aspect, a bioreactor may play a role in recellularization. In some cases, a bioreactor performs real time monitoring of certain parameters, such as pH, pO₂, pCO₂, temperature, electrolyte levels, glucose or lactate concentrations, and perfusion parameters, such as perfusion pressure and flow rates. The bioreactor may maintain conditions stable and adjustable, particularly during long-term culture. This monitoring further allows for calculation of other important parameters, such as vascular resistance, which can be useful for a controlled recellularization process. In an aspect, a resazurin-based assay can be used to investigate cellular viability and proliferation during reendothelialization, a noninvasive method for a more controlled recellularization process. In some cases, bioreactors may fulfil organ-specific requirements. For instance, recellularization of the lung may utilize tracheal access that facilitates the application of cells and medium via the tracheobronchial system or ventilation of the lung to observe gas exchange. On the other hand, tissue engineering of the heart may utilize electrical and biomechanical stimulation. In the tissue engineering of organs such as the liver, monitoring of the levels of albumin and coagulation factors is useful. Monitoring can be performed at any time, for example a measurement can be taken before infusion, during infusion, and after infusion. In some cases, a measurement can be taken from about 1 hour, 1 day, 2 days, 3 days, 4 days, 5 days, 6 days, 7 days, 8 days, 9 days, 10 days, 11 days, 12 days, 13 days, 14 days, 15 days, 17 days, 20 days, 22 days, 24 days, 28 days, 30 days, 2 months, 5 months, 7 months, 8 months, 10 months, or up to 1 year after infusion of a population of cells into an organ or portion thereof.

In some embodiments, a bioreactor can comprise means for increasing the level of oxygen in a culture media. In some embodiments, heightened oxygen levels can range from about 22% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, from about 95% to about 100%. In some embodiments, a level of increased oxygen can vary over culture time.

In some embodiments, elevated levels of oxygen in a media in which cells are cultured can result in a decrease in glucose consumption, delayed metabolic switching to glucogenesis, decrease in lactate production, decrease in ammonia concentrations, increased ammonia clearance rates, stability of mature phenotype, proliferation, or any combination thereof. In some embodiments, elevated levels of oxygen in a media in which cells, such as hepatocytes, are cultured in can result in a decrease in glucose consumption, phenotypic stability as measured by metabolic activity, decreased or delayed glucogenesis, secretion of coagulation factors, decrease in lactate production, decrease in ammonia concentrations, increased ammonia clearance, or any combination thereof. In some embodiments, the presence of heightened oxygen levels in a media in which one or more cells, such as hepatocytes, are cultured can extend the ability to clear ammonia. In some embodiments, a heightened oxygen level can be produced by direct oxygenation, in-line oxygenation, gas permeable materials, or any combination thereof. In some cases, direct oxygenation can comprise using a membrane oxygenating chamber. In some embodiments, direct oxygenating can comprise using a bubbler. In some embodiments, in-line oxygenation can comprise use of in-line oxygenators. In some embodiments, a media may be pumped by a peristaltic pump. In some embodiments, a media may be passed through gas permeable material allowing gaseous exchange through the material. In some embodiments, gaseous exchange may comprise oxygen exchange, nitrogen exchange, carbon dioxide exchange, or any combination thereof. In some embodiments, an at least partly permeable tubing may comprise silicone tubing. In some embodiments, silicone tubing may allow oxygen exchange, creating heightened oxygen levels in a media. In some embodiments, oxygen levels in the media can be heightened by a direct injection of a mixture of oxygen and one or more other gases. In some embodiments, one or more other gases can comprise nitrogen, carbon dioxide, or a combination of the two. In some embodiments, oxygen levels in the media can be heightened by an injection of a gas comprising about 40% oxygen, about 45% oxygen, about 50% oxygen, about 55% oxygen, about 60% oxygen, about 65% oxygen, about 70% oxygen, about 75% oxygen, about 80% oxygen, about 85% oxygen, about 90% oxygen, about 95% oxygen, or about 100% oxygen. In some embodiments, oxygen levels in the media can be heightened by an injection of about 100% pure oxygen. In some embodiments, heightened oxygen levels in the media can be facilitated by oxygen carrying molecules to increase access to cells within an isolated organ or portion thereof. In some cases, cells may comprise seeded hepatocytes. In some cases, an isolated organ or portion thereof may comprise an extracellular matrix (ECM) graft. In some cases, a media can be hyperoxygenated prior to seeding cells into an isolated organ or portion thereof. In some cases, a media can be hyperoxygenated prior to seeding hepatocytes into an ECM graft. In some cases, oxygen levels can be adjusted based on metrics. In some cases, metrics can be evaluated or adjusted and can comprise media glucose levels, lactate levels, pCO₂, pH, ammonia levels, pyruvate levels, other measurable parameters, and any combination thereof.

In some embodiments, disclosed herein may be a system comprising any of the compositions disclosed herein. In some embodiments, a system may comprise at least one of a bioreactor, pump, housing, tubing, oxygen permeable tubing, incubator, motor, computer, storage medium, biological safety cabinet, incubator, or any combination thereof. In some embodiments, cells are stored in an incubator. In some embodiments, an incubator can regulate temperature, gaseous concentration, humidity, and any combination thereof. In some embodiments, cells are cultured in a biological safety cabinet. In some embodiments, a biological safety cabinet can provide laminar airflow to prevent contamination of cells. In some embodiments, sterile techniques are performed to prevent contamination. In some embodiments, sterile techniques can include sterilizing surfaces and equipment with about 70% isopropyl alcohol, use of UV radiation, use of personal protective equipment such as gloves, lab coats, or body suits, or any combination thereof.

Provided herein can also be point-of-care facilities comprising for example a clean room, treatment rooms, patient rooms, good-manufacturing practices (GMP) rooms, and combinations thereof.

### EXEMPLARY EMBODIMENTS

Embodiment 1: An at least partially recellularized isolated organ or portion thereof comprising at least two different exogenous populations of cells engrafted thereon, wherein the at least partially recellularized isolated organ or portion thereof clears ammonia at a rate of at least 0.1 mmol per hour from a fluid perfused through the vasculature as measured by a flow meter.

Embodiment 2: An at least partially recellularized isolated organ or portion thereof comprising at least two exogenous populations of cells engrafted thereon, wherein the at least partially recellularized isolated organ or portion thereof comprises an at least partially intact vasculature that has a blood flow patency of at least 120 mL/min at about 15 mmHg as measured by a flow meter.

Embodiment 3: An at least partially recellularized isolated organ or portion thereof comprising at least two exogenous populations of cells engrafted thereon, wherein the at least partially recellularized isolated organ or portion thereof comprises an at least partially intact vasculature comprising a circulating fluid, and wherein the at least partially recellularized isolated organ or portion thereof maintains an ammonia concentration of the circulating fluid at a level of less than about 0.4 mM in a time period of about 24 hours as measured by an ammonia analyzer.

Embodiment 4: The at least partially recellularized isolated organ or portion thereof of any one of embodiment 1, or embodiment 3, wherein the fluid comprises blood.

Embodiment 5: The at least partially recellularized isolated organ or portion thereof of any one of embodiments 1 to embodiments 4, wherein the at least partially recellularized isolated organ or portion thereof is connected to a pump.

Embodiment 6: The at least partially recellularized isolated organ or portion thereof of any one of embodiments 1-5, wherein the at least two exogenous populations of cells engrafted thereon are allogeneic to the extracellular matrix of the at least partially recellularized isolated organ or portion thereof.

Embodiment 7: The at least partially recellularized isolated organ or portion thereof of any one of embodiments 1-5, wherein the at least two exogenous populations of cells engrafted thereon are autologous to the extracellular matrix of the at least partially recellularized isolated organ or portion thereof.

Embodiment 8: The at least partially recellularized isolated organ or portion thereof of any one of embodiments 1-5, wherein the at least two exogenous populations of cells engrafted thereon are xenogeneic to the extracellular matrix of the at least partially recellularized isolated organ or portion thereof.

Embodiment 9: An at least partially recellularized isolated organ or portion thereof comprising a population of engrafted exogenous cells, wherein a density of the population of the exogenous cells in a distal portion of the at least partially recellularized isolated organ or portion thereof comprises at most a 100% difference as compared to a density of the population of the exogenous cells in a proximal portion of the at least partially recellularized isolated organ or portion thereof, as measured by hematoxylin and eosin (H&E) staining of the population of the exogenous cells in the distal portion and the proximal portion of the at least partially recellularized isolated organ or portion thereof.

Embodiment 10: The isolated organ or portion thereof of any one of embodiments 1-9, further comprising a perfusion solution.

Embodiment 11: The method of embodiment 10, wherein the perfusion solution comprises at least 120 pO₂ mmHg as measured by a Jenway® Model 970 dissolved oxygen meter and electrode.

Embodiment 12: The isolated organ or portion thereof of embodiment 10, wherein the perfusion solution comprises a growth factor, an immune modulating agent, a coagulation modulating agent, an antibiotic, a preservative, or any combination thereof.

Embodiment 13: The isolated organ or portion thereof of embodiment 12, wherein the perfusion solution comprises the growth factor, and wherein the growth factor is selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 2 (BMP-2), Bone Morphogenic Protein 3 (BMP-3), Bone Morphogenic Protein 4 (BMP-4), Stromal Cell-Derived Factor 1 (SDF-1), Insulin like Growth Factor (IGF), Hepatocyte Growth Factor (HGF), and any combination thereof.

Embodiment 14: The isolated organ or portion thereof of embodiment 12, wherein the perfusion solution comprises the immune modulating agent, and wherein the immune modulating agent is a cytokine, a glucocorticoid, an interleukin-2 receptor (IL2R) antagonist, a leukotriene antagonist, or any combination thereof.

Embodiment 15: The isolated organ or portion thereof of any one of embodiments 1-14, wherein the first population of the exogenous cells comprises embryonic stem cells, induced pluripotent stem cells (iPSCs), umbilical cord blood cells, hepatocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof.

Embodiment 16: The isolated organ or portion thereof of any one of embodiments 1-15, wherein the at least partially recellularized isolated organ or portion thereof comprises an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, or an at least partially recellularized pancreas or portion thereof.

Embodiment 17: A system comprising the at least partially recellularized isolated organ or portion thereof of any one of embodiments 1-16 operatively coupled to a pump.

Embodiment 18: The system of embodiment 17, wherein the pump is a peristaltic pump or a vacuum pump.

Embodiment 19: The system of embodiment 17 or 18, wherein the system further comprises a cannula, a perfusion apparatus, a holding container, a tubing, a sensor, a thermometer, an electrode, a valve, a balloon, a pacemaker, a thermostat, a user interface, or any combination thereof.

Embodiment 20: The system of embodiment 19, wherein the system comprises the sensor, and wherein the sensor comprises a glucose sensor, an ammonia sensor, an oxygen sensor, a fluid sensor, a temperature sensor, a pressure sensor, or any combination thereof.

Embodiment 21: A method comprising introducing a second exogenous population of cells into an at least partially recellularized isolated organ or portion thereof comprising a first exogenous population of engrafted cells, wherein, prior to the introduction of the second population of exogenous cells, at least a portion of the first exogenous population of engrafted cells is functional as determined by: a. glucose consumption at a rate of at least about 10 mg/h; b. lactate production at a rate of at least about 30 mg/h; c. ammonia production at a rate of at least about 0.01 mmol/h; d. von Willebrand Factor production at a rate of at least about 0.1 ug/h; and f. any combination thereof.

Embodiment 22: The method of embodiment 21, wherein the at least partially recellularized isolated organ or portion thereof comprises an at least partially intact vasculature comprising a circulating fluid.

Embodiment 23: The method of embodiment 22, wherein the circulating fluid comprises blood or a fraction thereof.

Embodiment 24: The method of embodiment 22, wherein the circulating fluid comprises: i. a concentration of glucose of from about 0.5 g/L to about 4 g/L, ii. a concentration of oxygen of from about 120 mmHg to about 400 mmHg, iii. or any combination thereof.

Embodiment 25: The method of any one of embodiments 21-24, wherein at least 100% more blood perfusion rate is observed as measured by an external blood loop, compared to an otherwise comparable isolated organ or portion thereof generated by engrafting the second exogenous population of cells onto a decellularized organ or portion thereof that lacks the functional subset of the first exogenous population of cells.

Embodiment 26: The method of any one of embodiments 21-25, wherein the first exogenous population of engrafted cells comprises endothelial cells.

Embodiment 27: The method of embodiment 26, wherein the endothelial cells comprise human vein endothelial cells (HUVECs).

Embodiment 28: The method of any one of embodiments 21-27, wherein the second exogenous population of cells comprises embryonic stem cells, induced pluripotent stem cells (iPSCs), umbilical cord blood cells, hepatocytes, cholangiocytes, podocytes, mesangial cells, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), iPSC derived endothelial cells, differentiated stem cells or any combination thereof.

Embodiment 29: The method of any one of embodiments 21-28, wherein at least a portion of the second exogenous population of cells comprises liver specific cells or kidney specific cells, wherein the exogenous population of cells are liver specific cells and are hepatocytes or cholangiocytes, and wherein the exogenous population of cells are kidney specific cells and are podocytes or mesangial cells.

Embodiment 30: The method of any one of embodiments 21-29, wherein the introducing is via a cannula.

Embodiment 31: The method of any one of embodiments 21-30, wherein at least 25% more of any one of glucose consumption, lactate consumption, oxygen consumption, ribose consumption, and glycogen production is observed in the at least partially recellularized isolated organ or portion thereof as compared to a comparable isolated organ or portion thereof generated by a comparable method absent the functional subset of the first exogenous population of engrafted cells before the introduction of the second exogenous population of cells.

Embodiment 32: The method of any one of embodiments 21-31, wherein the portion of the first exogenous population of engrafted cells comprises at least 5% of the first exogenous population of engrafted cells.

Embodiment 33: The method of any one of embodiments 21-32, wherein the at least partially recellularized isolated organ or portion thereof is an at least partially recellularized liver or portion thereof, and wherein the second exogenous population of cells are perfused into the liver or portion thereof via a hepatic vein.

Embodiment 34: The method of any one of embodiments 21-32, wherein the at least partially recellularized isolated organ or portion thereof is an at least partially recellularized liver or portion thereof, and wherein the second exogenous population of cells are perfused into the liver or portion thereof via a bile duct.

Embodiment 35: The method of any one of embodiments 21-34, wherein the second exogenous population of cells comprises hepatocytes.

Embodiment 36: The method of any one of embodiments 21-35, wherein at least one of the populations of exogenous cells are introduced by perfusing a recellularization solution into the at least partially recellularized isolated organ or portion thereof while the at least partially recellularized isolated organ or portion thereof is at least partially submerged in a liquid that comprises the recellularization solution.

Embodiment 37: The method of embodiment 36, wherein the perfusing is via a cannula.

Embodiment 38: The method of embodiment 37, wherein the perfusing is antegrade.

Embodiment 39: The method of embodiment 37, wherein the perfusing is retrograde.

Embodiment 40: A method comprising: a) determining a concentration of a factor circulating in an at least partially recellularized isolated organ or portion thereof comprising a first population of cells engrafted thereon; and b) introducing into the at least partially recellularized isolated organ or portion thereof a second population of cells, wherein the first population of cells and the second population of cells are different, and wherein at least one of the first population of cells or the second population of cells are exogenous to the at least partially recellularized isolated organ or portion thereof.

Embodiment 41: The method of embodiment 40, wherein the factor is glucose, lactate, ammonia, oxygen, ribose, or glycogen.

Embodiment 42: The method of embodiment 40 or 41, wherein the first population of cells comprises endothelial cells.

Embodiment 43: The method of any one of embodiments 40-42, wherein the second population of cells comprises embryonic stem cells, induced pluripotent stem cells (iPSCs), umbilical cord blood cells, hepatocytes, cholangiocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), iPSC derived endothelial cells, differentiated stem cells, or any combination thereof.

Embodiment 44: The method of any one of embodiments 40-43, wherein the at least partially recellularized isolated organ or portion thereof is a liver, a kidney, a heart, a lung, a bowel, a skeletal muscle, a bone, a uterus, a bladder, a spleen, a brain, and a pancreas.

Embodiment 45: The method of any one of embodiments 40-44, wherein the at least partially recellularized isolated organ or portion thereof is cultured in hyperoxic conditions following the introduction of the second population of cells.

Embodiment 46: The method of embodiment 45, wherein the hyperoxic conditions comprise an oxygen concentration over 21%, 22%, 23%, or 24% pO₂ 140 mmHg as measured by a Jenway® Model 970 Dissolved Oxygen Meter and Electrode.

Embodiment 47: A method comprising implanting the at least partially recellularized isolated organ or portion thereof of any one of embodiments 1-16, into a subject.

Embodiment 48: The method of embodiment 47, wherein the subject has liver disease, hypertension, diabetes, heart failure, lung disease, or kidney disease.

Embodiment 49: The method of embodiment 48, wherein the subject has the liver disease, and wherein the liver disease is cirrhosis, nonalcoholic steatohepatitis, hepatocellular carcinoma, metabolic disease, or any combination thereof.

Embodiment 50: The method of any one of embodiments 47-49, further comprising administering an immunosuppressive condition to the subject.

Embodiment 51: The method of any one of embodiments 47-50, wherein the subject exhibits an intercranial pressure of less than 20 mmHg for a duration of at least two hours during a time period of at least 24 hours after the implanting.

Embodiment 52: A method comprising implanting an isolated at least partially decellularized, at least partially re-endothelialized organ into a subject, wherein said isolated at least partially decellularized, at least partially re-endothelialized organ retains vascular patency for a time period of at least 9 days.

Embodiment 53: The method of embodiment 52, wherein said at least partially decellularized, at least partially re-endothelialized organ has been at least partially re-endothelialized by contacting with cells that are exogenous to said isolated organ.

Embodiment 54: A composition comprising: (a) an isolated at least partially decellularized, at least partially recellularized organ; and (b) a circulatory system comprising a liquid, wherein said isolated at least partially decellularized, at least partially recellularized organ substantially retains liquid for at least 9 days.

Embodiment 55: A method comprising revascularizing an isolated at least partially decellularized organ with a cell, the method comprising perfusing said cells through a vein of said organ, then perfusing said cells through an artery of said organ.

Embodiment 56: A system comprising: (a) an isolated at least partially decellularized, at least partially re-endothelialized organ, (b) a circulatory system at least partially connected to said isolated at least partially decellularized, at least partially re-endothelialized organ, and (c) a liquid circulating through said at least partially decellularized, at least partially re-endothelialized organ, and (d) a sensor configured to measure a component of said media.

Embodiment 57: The method of embodiment 56, wherein said system is configured to increase a volume of media in said system in response to a measurement of said sensor.

Embodiment 58: The method of embodiment 56, wherein said component measured by said sensor comprises glucose, glutamine, glutamate, ammonia, lactate dehydrogenase (LDH), or any combination thereof.

Embodiment 59: The method of embodiment 56, wherein said sensor comprises a glucose sensor.

Embodiment 60: The method of embodiment 59, wherein said measurement comprises a glucose level of said liquid falling below 0.2 g/L.

Embodiment 61: The method of embodiment 60, wherein said change of volume comprises an increase of volume in said system.

Embodiment 62: The method of embodiment 56, wherein said sensor comprises an ammonia sensor.

Embodiment 63: The method of embodiment 62, wherein said measurement comprises an ammonia level of said liquid increasing above 1 mM.

Embodiment 64: The method of embodiment 63, wherein said change of volume comprises an increase of volume in said system.

Embodiment 65: A method of at least partially treating kidney failure in a subject in need thereof, comprising grafting an at least partially recellularized kidney onto a circulatory system of the subject, wherein the at least partially recellularized kidney comprises at least a portion of an at least partially intact porcine kidney extracellular matrix comprising xenogeneic or allogeneic glomerular cells engrafted thereon prior to the grafting, wherein the grafting: (a) reduces a level of hematocrit in the blood of the subject, relative to a level of hematocrit in the blood prior to the grafting, (b) reduces an effluent protein concentration of the blood of the subject, relative to a protein concentration in the blood prior to the grafting; (c) is sufficient to produce an effluent flow rate that is comparable to a native porcine kidney; or (d) any combination thereof; thereby at least partially treating the kidney failure in the subject.

Embodiment 66: The method of embodiment 65, wherein the glomerular cells comprise podocytes.

Embodiment 67: The method of embodiment 65, wherein the glomerular cells comprise mesangial cells.

Embodiment 68: The method of any one of embodiments 65-67, wherein the glomerular cells comprise human cells.

### Examples

### Example 1: Perfusion Decellularization of Organs or Portions Thereof

### Part 1: Cannulation

Hearts were systemically heparinized with 400 U of heparin/kg of donor (for example pig, canine, human, primate, and sheep). Following heparinization, the heart and the adjacent large vessels were carefully removed. The heart was placed in a physiologic saline solution (0.9%) that optionally contained heparin (2000 U/ml) and was held at 5° C. until further processing. Under sterile conditions, the connective tissue was removed from the heart and the large vessels. The inferior venae cava and the left and right pulmonary veins were ligated distal from the right and left atrium using monofil, non-resorbable ligatures.

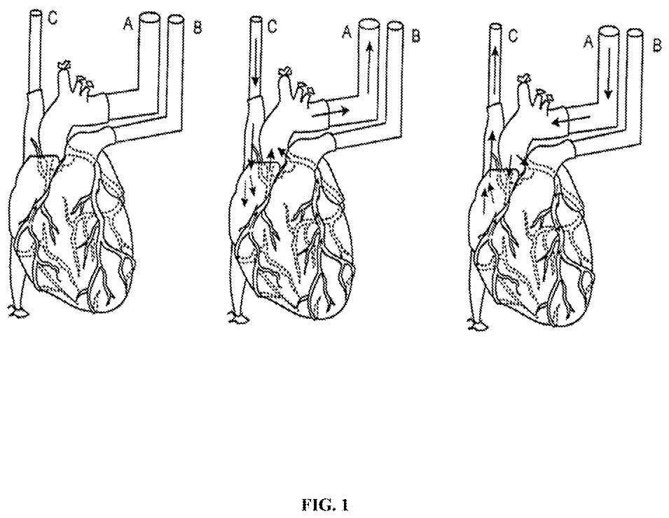

The heart was mounted on a decellularization apparatus for perfusion (FIG. 1). The descending thoracic artery was cannulated to allow retrograde coronary perfusion (FIG. 1, Cannula A). The branches of the thoracic artery (e.g., brachiocephalic trunk, left common carotid artery, left subclavian artery) were ligated. The pulmonary artery was cannulated before its division into the left and right pulmonary artery (FIG. 1, Cannula B). The superior vena cava was cannulated (FIG. 1, Cannula C). This configuration allowed for both retrograde and antegrade coronary perfusion. When positive pressure was applied to the aortic cannula (A), a perfusion occurred from the coronary arteries through the capillary bed to the coronary venous system to the right atrium and the superior caval vein (C). When positive pressure was applied to the superior vena cava cannula (C), perfusion occurred from the right atrium, the coronary sinus, and the coronary veins through the capillary bed to the coronary arteries and the aortic cannula (A).

### Part II: Decellularization

### Basic Protocol

After the heart is mounted on the decellularization apparatus, antegrade perfusion was started with cold, heparinized, calcium-free phosphate buffered solution containing 1-5 mmol adenosine per L perfusate to reestablish constant coronary flow. Prior to the perfusion, the buffered solution was placed under high vacuum for 30 minutes with vigorous stirring using a stir bar for 30 minutes at 25° C.

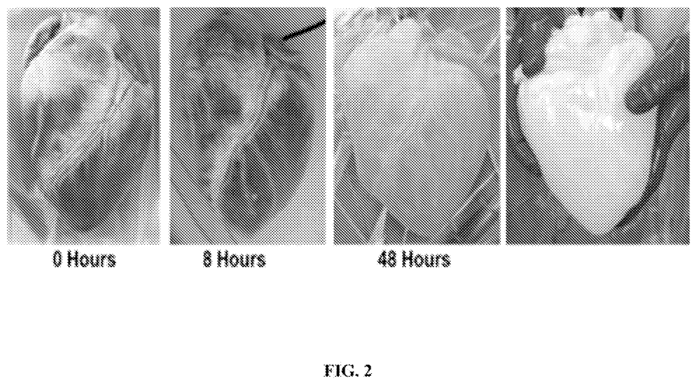

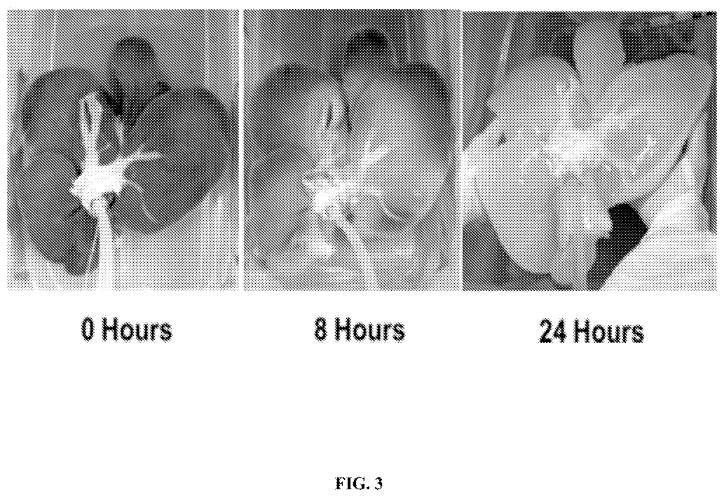

Coronary flow was assessed by measuring the coronary perfusion pressure and the flow, and by calculating coronary resistance. After 15 minutes of stable coronary flow, the detergent-based decellularization process was initiated utilizing a degassed decellularization solution. In some cases, the heart or portion thereof was perfused antegrade with a degassed detergent solution. After perfusion, the heart or portion thereof was flushed with a degassed buffer (e.g., PBS) retrograde. The heart was then perfused with a degassed PBS buffer containing antibiotics and then a degassed PBS buffer containing DNase I. The heart was then perfused with 1% benzalkonium chloride to reduce microbial contamination and to prevent future microbial contamination and was then perfused with PBS to wash the isolated organ of any residual cellular components, enzymes, or detergent. Exemplary solid organs that were decellularized are shown in FIG. 2 (porcine heart) and FIG. 3 (porcine liver).

### PEG Decellularization

Hearts were washed in 200 ml PBS containing 100 U/ml penicillin, 0.1 mg/ml Streptomycin, and 0.25 μg/ml Amphotericin B with no recirculation. Hearts were then decellularized with 35 ml of degassed polyethylene glycol (PEG; 1 g/ml) for up to 30 minutes with manual recirculation. The degassed polyethylene glycol was prepared by placing the polyethylene glycol solution under a vacuum pump. The isolated organ was then washed with 500 ml degassed PBS for up to 24 hours using a pump for recirculation. The washing step was repeated at least twice for at least 24 hours each time. Hearts were exposed to 35 ml DNase I (70 U/ml) for at least 1 hour with manual recirculation. The isolated organs were washed again with 500 ml degassed PBS for at least 24 hours.

### Triton X and Trypsin Decellularization

Hearts were washed in 200 ml PBS containing 100 U/ml Penicillin, 0.1 mg/ml Streptomycin, and 0.25 μg/ml Amphotericin B for at least about 20 minutes with no recirculation. Hearts were then decellularized with a degassed aqueous solution of 0.05% Trypsin for 30 min followed by perfusion with 500 ml degassed PBS containing 5% Triton-X and 0.1% ammonium-hydroxide for about 6 hours. Both solutions were prepared by dissolving a detergent with water previously degassed. Hearts were perfused with degassed deionized water for about 1 hour, and then perfused with degassed PBS for 12 h. Hearts were then washed 3 times for 24 hours each time in 500 ml degassed PBS using a pump for recirculation. The hearts were perfused with 35 ml DNase I (70 U/ml) for 1 hour with manual recirculation and washed twice in 500 ml PBS for at least about 24 hours each time using a pump for recirculation.

### SDS Decellularization

Hearts were washed in 200 ml PBS containing 100 U/ml Penicillin, 0.1 mg/ml Streptomycin, and 0.25 μg/ml Amphotericin B for at least about 20 mins with no recirculation. The hearts were decellularized with 4000 ml degassed water containing 1% SDS for at least about 6 hours using a pump for recirculation. The degassed water was prepared by heating deionized water to about 65° C. under low vacuum for about an hour. The hearts were then washed with degassed deionized water for about 1 hour and washed with degassed PBS for about 12 hours. The hearts were washed three times with 500 ml degassed PBS for at least about 24 hours each time using a pump for recirculation. The heart was then perfused with 35 ml DNase I (70 U/ml) for about 1 hour using manual recirculation, and washed three times with 500 ml degassed PBS for at least about 24 hours each time using a pump for recirculation.

Livers were washed in 2000 ml PBS, disinfected with PAA, and perfused with 2000 ml of degassed 0.9% saline. The livers were decellularized with a total of about 10 L degassed water containing about 0.6% SDS for at least about 6 hours using a pump for a series of single flow through and recirculation baths. The degassed water was prepared by passing deionized water through a vacuum filter designed to degas the solution. The livers were then washed with degassed deionized water for about 1 hour and washed with degassed PBS for about 12 hours. The livers were washed three times with 2000 ml degassed PBS for at least about 2 hours each time using a pump for recirculation.

### Triton X Decellularization

Hearts were washed with 200 ml PBS containing 100 U/ml Penicillin, 0.1 mg/ml Streptomycin, and 0.25 μg/ml Amphotericin B for at least about 20 mins with no recirculation. Hearts were then decellularized with 500 ml degassed water containing 5% Triton X and 0.1% ammonium hydroxide for at least 6 hours using a pump for recirculation. Degassed buffers were prepared as described above for PEG decellularization. Hearts were then perfused with degassed deionized water for about 1 hour and then with degassed PBS for about 12 hours. Hearts were washed by perfusing with 500 ml degassed PBS 3 times for at least 24 hours each time using a pump for recirculation. Hearts were then perfused with 35 ml DNase I (70 U/ml) for about 1 hour using manual recirculation, and washed three times in 500 ml degassed PBS for about 24 hours each time.

For initial experiments, the decellularization apparatus was set up within a laminar flow hood. Hearts were perfused at a coronary perfusion pressure of 60 cm H₂O. Although not required, the hearts described in the experiments above were mounted in a decellularization chamber and completely submerged and perfused with degassed PBS containing antibiotics for 72 hours in recirculation mode at a continuous flow of 5 ml/min to wash out as many cellular components and detergent as possible.

Successful decellularization were shown by lack of myofilaments and nuclei in histologic sections. Successful preservation of vascular structures was assessed by perfusion with 2% Evans Blue prior to embedding tissue sections. In some cases, decellularization took place when a heart was first perfused antegrade with an ionic detergent (1% sodium-dodecyl-sulfate (SDS), approximately 0.03 M) dissolved in deionized H₂O at a constant coronary perfusion pressure and then perfused antegrade with a non-ionic detergent (1% Triton X-100) to remove the SDS and presumably to renature the extracellular matrix (ECM) proteins. Intermittently, the heart was perfused retrogradely with phosphate buffered solution to clear obstructed capillaries and small vessels.

### Peracetic Acid (PAA) Decellularization

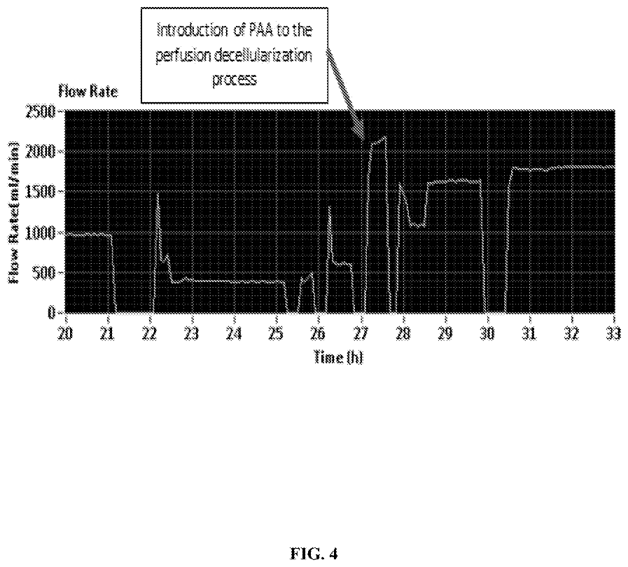

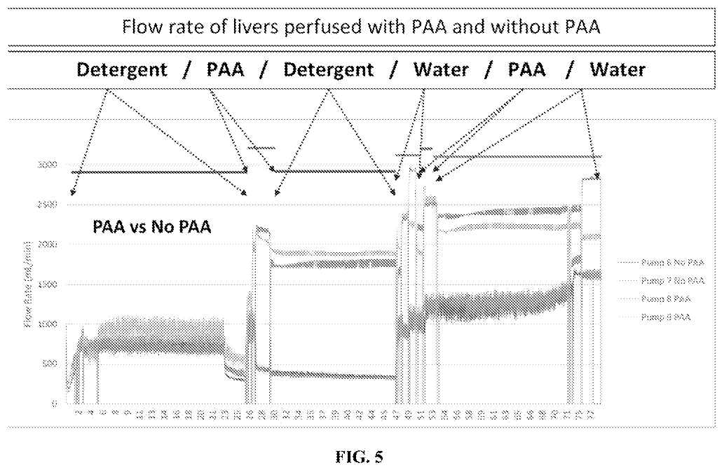

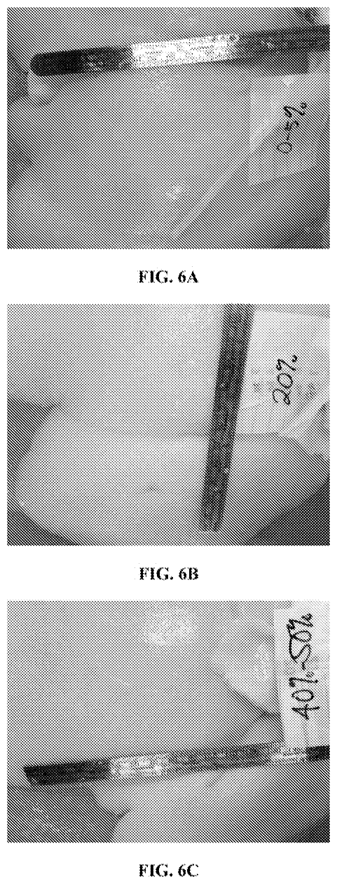

A liver from a pig was obtained and cannulated. The native vasculature of a porcine liver was cannulated and perfused with a mild detergent solution in order to decellularize the liver. The detergent solution further contained peracetic acid. It was observed that after the detergent solution containing peracetic acid was introduced to the isolated organ at a set pressure (e.g., 12 mmHg), the flow rate increased (e.g., increased to at least 500 mL/min, 1000 mL/min, 1500 mL/min, 2000 mL/min, 2500 mL/min, or greater, as shown in FIG. 4, and the decellularization process decreased

This experiment showed that the use of a peracid (in this case peracetic acid (PAA)) at a concentration of about 50-2000 ppm resulted in an increase in the flow rate during perfusion decellularization at a predetermined pressure as compared to a comparable decellularization process without using a peracid, as shown in FIG. 5. This increase in the flow rate at a set pressure occurred during the perfusion with a peracid solution (PAA) and was retained in subsequent perfusion steps, even in steps that did not include a peracid. This increase in flow rate resulted in a more complete decellularization of the isolated organ without affecting the integrity matrix.

Overall, the results showed that more cells were removed in a shorter period of time and that the increase in flow rate resulted in a purer and more complete decellularization of the isolated organ. Isolated organs are dense with vascular capillaries with most cells located in close proximity to a capillary. Thus, perfusion of a cannulated isolated organ with a solution containing a peracid and the subsequent increase in the flow rate resulted in an increased effective surface area of the detergent and a decreased time required to expel the native cellular material through the venous system. The results also showed that the increase in the flow rate or the addition of a peracid during decellularization did not significantly affect or compromise the integrity matrix.

### Example 2: Evaluation of Decellularized Isolated Organs

The presentation of a non-soluble white particulate is noticed during the perfusion decellularization of isolated whole organs or portions thereof with some detergents such as SDS. Particulate forms and then may become trapped in the isolated organ or portion thereof during the decellularization. Reducing particulate formation may comprise significantly decreasing the amount of trace detergents or detergent remaining after decellularization. Reducing particulate formation may increase the ability to recellularize the isolated organ. Reducing particulate formation may decrease cytotoxicity of the decellularized matrix to introduced cells. Particulate may be formed by an insoluble interaction between native proteins and detergents. Reducing particulate may be performed by using solutions, controlling a mammal's eating habits, or their combination.

### Saline Solution

During isolated organ decellularization, isolated organs were immediately treated with detergent. The addition of saline was found to inhibit or prevent particulate formation. After excision from a mammal, isolated organs were flushed with 0.9% saline or PBS and then disinfected with peracetic acid. The isolated organs were primed with 0.9% saline prior to decellularization with detergent and placed into a 0.9% saline or PBS bath prior to detergent perfusion.

In particular, the porcine liver was washed in 0.9% saline or PBS followed by a peracetic acid wash. The washed liver was then submerged in a 0.9% saline or PBS bath. For decellularization, the liver was decellularized with 5000 ml water containing 1% SDS for at least about 6 hours using a pump for recirculation. The liver was then washed with deionized water for about 1 hour and washed with PBS for about 12 hours. The livers were washed three times with 500 ml PBS for at least about 24 hours each time using a pump for recirculation. The liver was then perfused with 35 ml DNase I (70 U/ml) for about 1 hour using manual recirculation, and washed three times with 500 ml PBS for at least about 24 hours each time using a pump for recirculation.

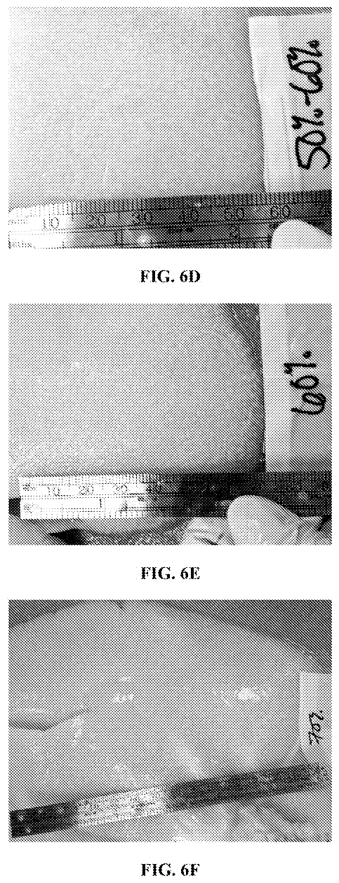

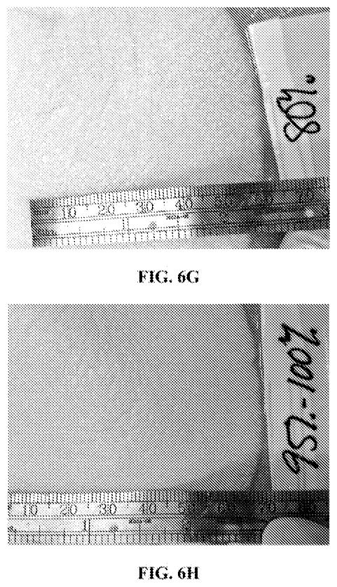

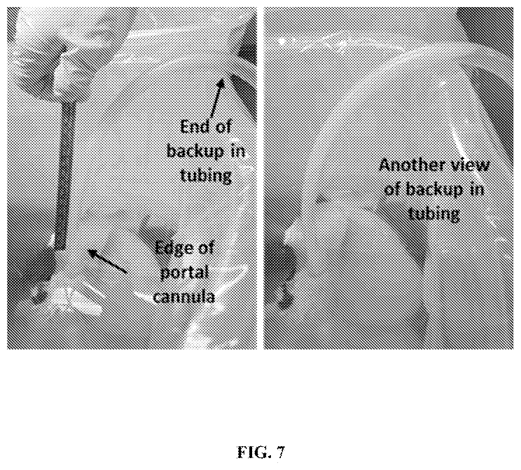

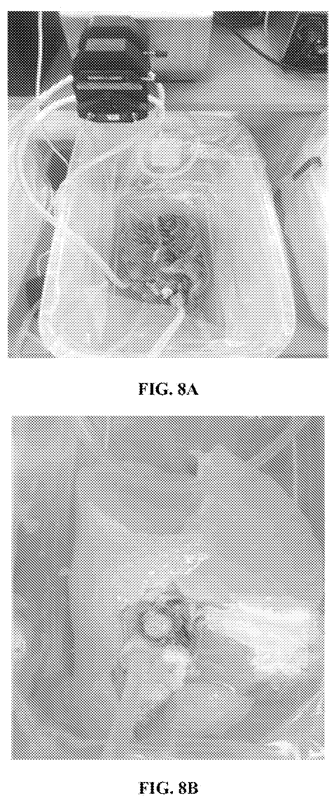

FIG. 6A depicts a porcine liver after decellularization having a particulate percent from 0-5%. FIG. 6B depicts a porcine liver after decellularization having a particulate percent of 20%. FIG. 6C depicts a porcine liver after decellularization having a particulate percent from 40-50%. FIG. 6D depicts a porcine liver after decellularization having a particulate percent from 50-60%. FIG. 6E depicts a porcine liver after decellularization having a particulate percent of 60%. FIG. 6F depicts a porcine liver after decellularization having a particulate percent of 70%. FIG. 6G depicts a porcine liver after decellularization having a particulate percent of 80%. FIG. 6H depicts a porcine liver after decellularization having a particulate percent from 95-100%. FIG. 7 depicts an elution of particulate into a tubing connected to a portal vein cannula of a liver with visual particulate undergoing perfusion decellularization when perfusion is stopped for 10 minutes after about 24 hours of perfusion decellularization. FIG. 8A depicts decellularization of a 6-month old adult porcine lung undergoing perfusion decellularization over a period of 48 hours. A native structure and vasculature are preserved after decellularization. FIG. 8B depicts decellularization after 48 hours, a lung is completely decellularized.

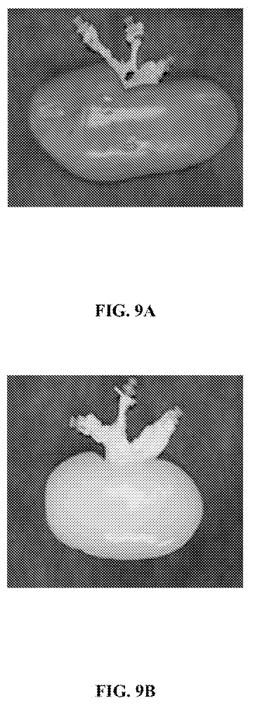

FIG. 9A depicts decellularization of an adult porcine kidney over 48 hours. A 6-month-old porcine kidney undergoing perfusion decellularization over a period of 48 hours. A native structure and vasculature are preserved after decellularization. FIG. 9B depicts decellularization of a kidney after 48 hours, a kidney is completely decellularized.

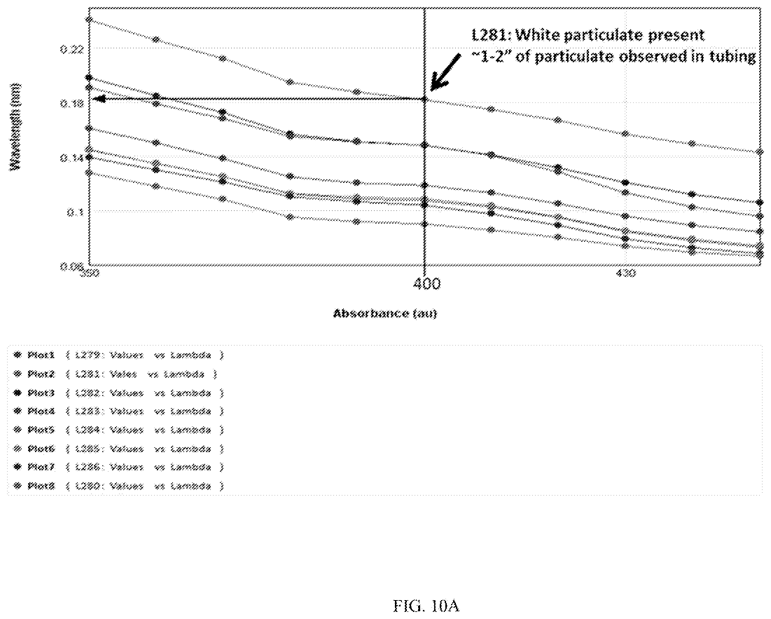

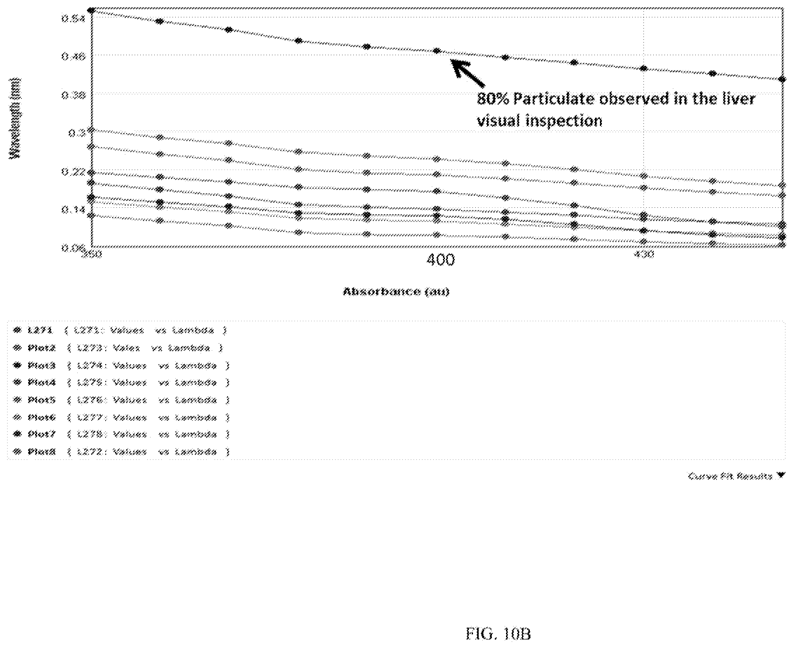

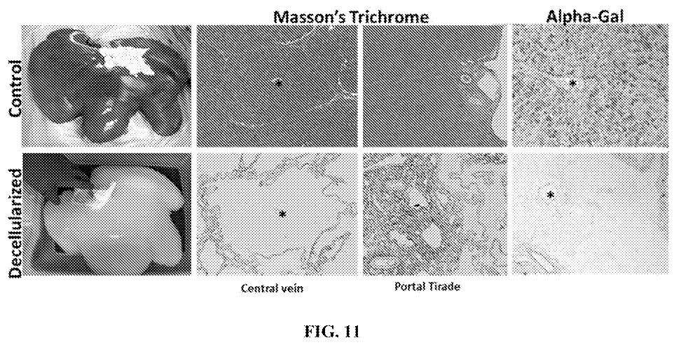

FIG. 10A shows an absorbance profile of a solution surrounding a liver with visible particulate (L281) after a 30 minute re-circulating water rinse. FIG. 10B shows an absorbance profile of effluent in a solution surrounding a liver with visible particulate L274 after 15 minutes of a re-circulating water wash.

To demonstrate intact vascular structures following decellularization, an at least partially decellularized heart was stained via Langendorff perfusion with Evans Blue to stain vascular basement membrane and quantify macro- and micro-vascular density. Further, polystyrene particles were perfused into and through some hearts to quantify coronary volume, the level of vessel leakage, and to assess the distribution of perfusion by analyzing coronary effluent and tissue sections. A combination of three criteria were assessed and compared to isolated non-decellularized heart: 1) an even distribution of polystyrene particles, 2) significant change in leakiness at some level 3) microvascular density.

Fiber orientation was assessed by the polarized-light microscopy technique of Tower et al. (2002, Fiber alignment imaging during mechanical testing of soft tissues, Ann Biomed Eng., 30(10):1221-33), which was applied in real-time to a sample subjected to uniaxial or biaxial stress. During Langendorff perfusion, basic mechanical properties of an at least partially decellularized ECM were recorded (compliance, elasticity, burst pressure) and compared to freshly isolated hearts.

Histological Evaluation

To evaluate the cellular content and histoarchitecture, representative samples of both native and decellularized kidneys, hearts and livers were processed for hematoxylin and eosin (H&E). The samples were fixed in 10% neutral buffered formalin solution at 4° C. for 24 h. Subsequently, these were washed in distilled water, dehydrated in graded alcohol, embedded in paraffin and sectioned (5 mm) for staining. Tissue slides were stained with H&E following standard protocols. To exclude the presence of nuclear materials, the native kidneys and decellularized kidney scaffolds were processed for DNA-binding fluorescent staining by DAPI (40, 6-diamidino-2-Phenylindole) staining as shown in FIG. 11. Examination of stained sections were performed using an immunofluorescent microscope.

Scanning Electron Microscopy (SEM)

For assessment of ultrastructure after the decellularization step, specimens of native kidneys and decellularized kidneys were processed for scanning electron microscopy. The specimens were washed in distilled water and fixed in cold 2.5% glutaraldehyde for 2 h at 48° C. Following washing with PBS to remove the residual glutaraldehyde, the samples were dehydrated by increasing concentrations of ethanol (30% ethanol, 50% ethanol, 70% ethanol, 90% ethanol, and 100% ethanol) for 20-30 min at room temperature. The samples were critical point dried using CO₂ and finally mounted on aluminum stubs using sticky carbon taps for imaging using a scanning electron microscope.

### Vascular Network Imaging

To evaluate the vascular network and kidney capsule integrity, radio-opaque contrast solution was perfused through the renal artery into the native and decellularized kidneys, contrast-radiography was employed 10 min after contrast reagent perfusion.

### Quantification of Extracellular Matrix (ECM) Components

To check the effect of the decellularization process on ECM components, samples of native and decellularized renal tissues were digested in PBS containing 50 mg mL proteinase K at 56° C. overnight. Then, the lysates were heat-inactivated at 90° C. for 10 min and centrifuged at 13,000×g for 10 min. Supernatants were collected and assayed for protein concentration using the BCA Protein Assay. GAGs, collagen, and elastin levels were quantified in the lysate using the Blyscan sGAGs Assay Kit, the Sircol Soluble Collagen Assay Kit, and Fastin Elastin Assay Kit, respectively according to the manufacturer's instructions.

### Mechanical Testing

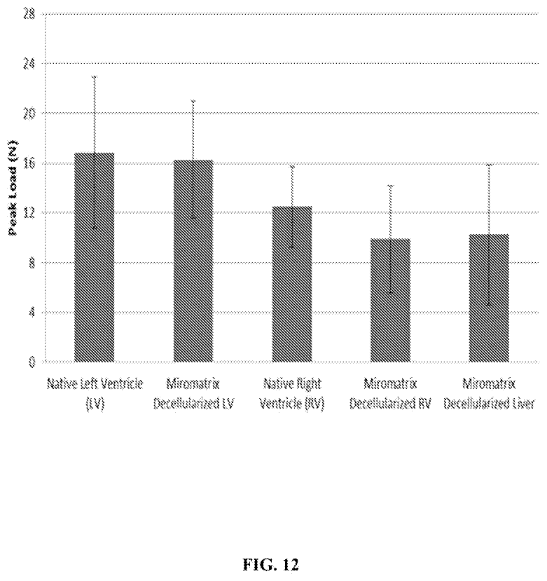

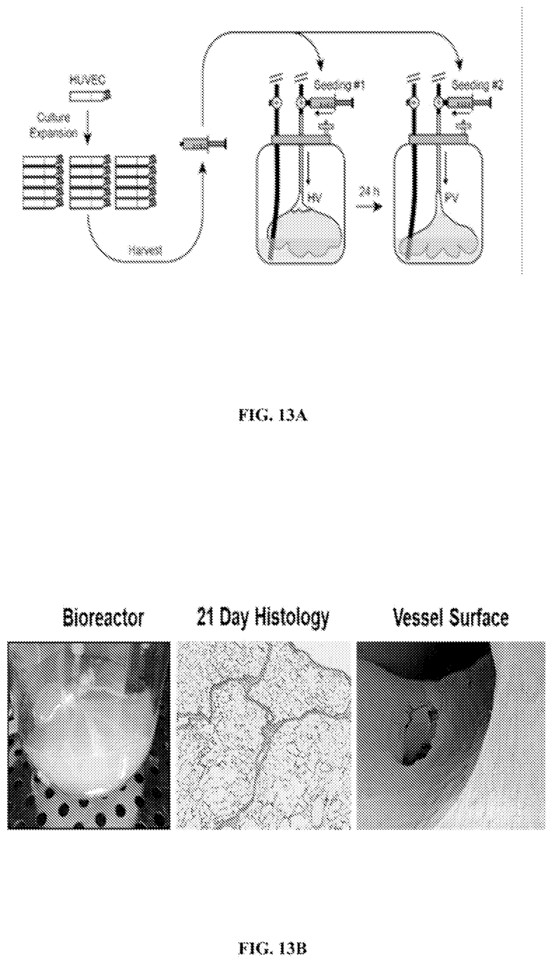

Crosses of myocardial tissue were cut from the left ventricle of rats so that the center area was approximately 5 mm×5 mm and the axes of the cross are aligned in the circumferential and longitudinal directions of the heart. The initial thickness of the tissue crosses were measured by a micrometer. Crosses were also cut from decellularized rat left ventricular tissue in the same orientation and with the same center area size. In addition, the mechanical properties of fibrin gels were tested, another tissue engineering scaffold used in engineering vascular and cardiac tissue. Fibrin gels were cast into cross-shaped molds with a final concentration of 6.6 mg of fibrin/ml. All samples were attached to a biaxial mechanical testing machine via clamps, submerged in PBS, and stretched equibiaxially to 40% strain. In order to probe the static passive mechanical properties accurately, the samples were stretched in increments of 4% strain and allowed to relax at each strain value for at least 60 seconds. FIG. 12 depicts a plot of the peak load for the samples. Forces were converted to engineering stress by normalizing the force values with the cross-sectional area in the specific axis direction (5 mmx initial thickness). Engineering stress was calculated as the displacement normalized by the initial length. In order to compare the data between the two axes as well as between sample groups, a tangential modulus is calculated as follows: [T(ε=40% strain)−T(ε=36% strain)]/4% strain, where T is engineering stress and E is engineering strain. The values for the tangential modulus are averaged and compared between the two axes (circumferential and longitudinal) as well as between groups.

### Recellularization of Livers

HUVEC cell culture and seeding of decellularized liver constructs: Human umbilical vein endothelial cells were cultured in antibiotic-free EGM-2 medium in tissue culture flasks at 37° C. and 5% CO₂ and passaged with 0.25% trypsin at 90-100% confluency according to manufacturer's protocol. The highest passage used for seeding liver grafts was passage 11. Decellularized porcine livers utilizing degassed decellularization were placed in a custom bioreactor containing 800 ml of media cell, connected to the perfusion inlet via the suprahepatic vena cava, and perfused at 12 mmHg with culture media prior to seeding. HUVECs were resuspended in 100 ml of media and seeded through the suprahepatic vena cava. The infused cell suspension was left under static conditions for one hour and then perfusion was restarted. After 24 hours, perfusion was changed from the suprahepatic vena cava to the portal vein and the seeding protocol was repeated. Re-reendothelialized grafts were maintained in a continuous perfusion loop with metabolites (glucose, lactate, glutamine, glutamate and ammonia) monitored daily in collected media samples using a BioProfile FLEX analyzer. Culture media was exchanged, and the volume increased depending on the rate of glucose depletion in the circulating medium to ensure 24-hour glucose levels above 500 mg/L.

### Histological Analysis

Tissue samples were perfused with PBS and fixed with 10% Neutral Buffered Formalin (VWR 16004-128). Fixed tissues were paraffin embedded, sectioned and stained using standard histologic techniques. FIG. 13A depicts a representation of endothelial seeding used in the histological analysis. FIG. 13B depicts representative images of the bioreactor, histology after 21 days, and an exemplary vessel surface.

Recellularization of Kidneys with Whole Liver Isolation

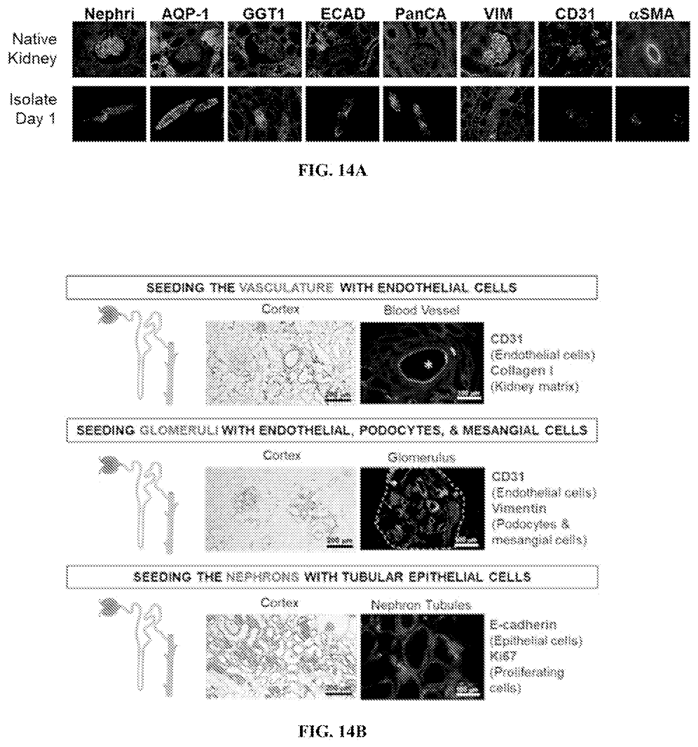

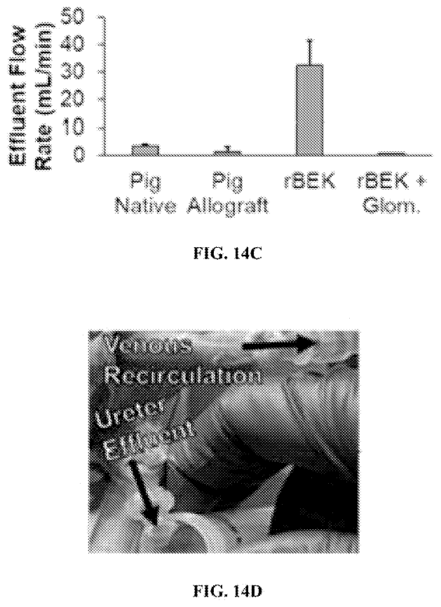

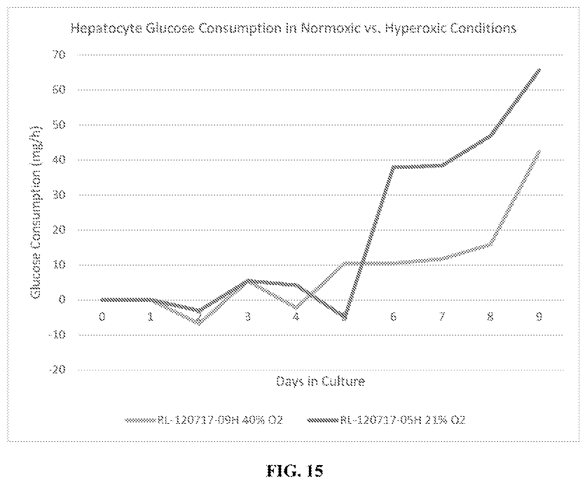

Decellularized porcine kidneys utilizing degassed decellularization were placed in a custom bioreactor containing 800 ml of cell media, connected to the perfusion inlet via the renal artery, and perfused at 12 mmHg with culture media prior to seeding, see FIG. 14B. Kidney isolates were resuspended in 100 ml of media and seeded through the ureter. The infused cell suspension was left under static conditions for one hour and then perfusion was restarted. Recellularized kidneys were maintained in a continuous perfusion loop with metabolites (glucose, lactate, glutamine, glutamate and ammonia) monitored daily in collected media samples using a BioProfile FLEX analyzer. Culture media was exchanged, and the volume increased depending on the rate of glucose depletion in the circulating medium to ensure 24-hour glucose levels above 500 mg/L. As depicted in FIG. 14A, immunofluorescent staining demonstrated the distribution and engraftment of native kidney cells throughout the kidney in the proper location. The effluent flow rate of the same is provided in FIG. 14C and effluent generation shown in FIG. 14D.

### Example 3: Use of Oxygen to Retain Functional Hepatocytes and to Maintain Ammonia Clearance in Seeded Extracellular Matrix Grafts

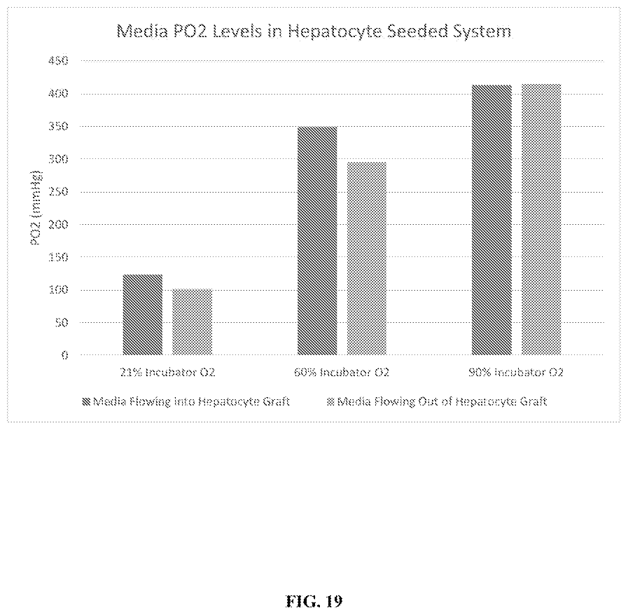

Decellularized whole livers were placed into bioreactors and continuously perfused with cell culture media. Freshly harvested hepatocytes were seeded into the livers via the hepatic vein (4×10⁹ porcine hepatocytes) and then following seeding for 4-14 hrs., perfusion was changed to the portal vein and continued for the duration of the study. Media was changed every 24 to 48 hr. depending on the culture conditions and metabolic activity of the organ. Following seeding, the recellularized liver grafts were maintained at a pressure of 12 mmHg for the duration of the study and placed in normoxic or hyperoxic conditions with gas permeable tubing to increase oxygen levels in the circulating media. Analysis of normoxic (21%), and hyperoxic (60% and 90%) demonstrated an increase in retained oxygen levels from ˜120 mmHg to 350 mmHg and 400 mmHg respectively (FIG. 19). Measurement of the oxygen levels in the outflow (immediately exiting the liver via the hepatic vein) demonstrated no appreciative reduction in oxygen levels of the conditions (FIG. 19), demonstrating that under normoxic conditions the recellularized liver graft isn't considered in a hypoxic condition.

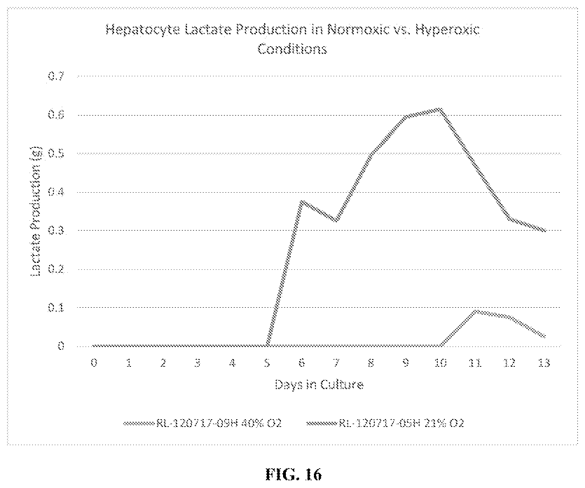

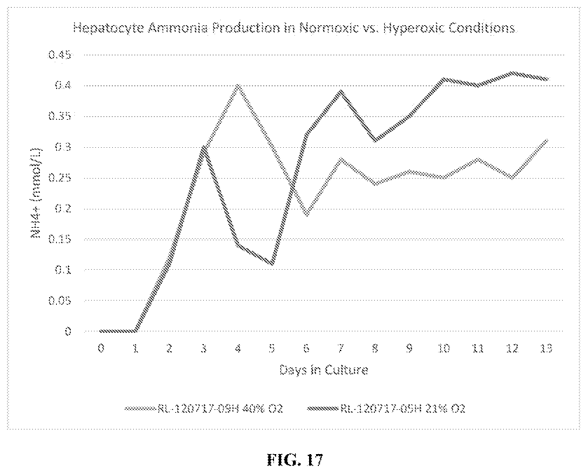

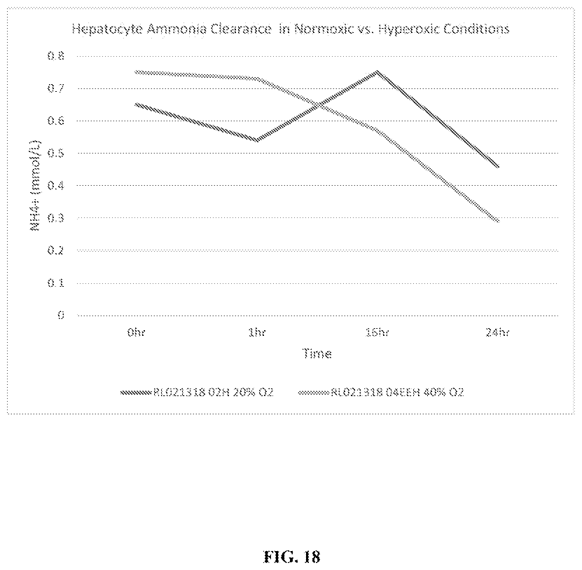

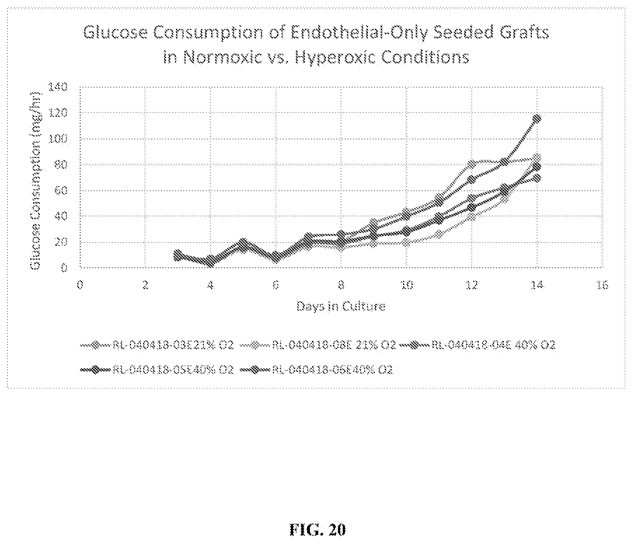

The increase in oxygen levels in the media resulted in a delay of the observed increased in glucose consumption rate compared to the normoxic condition (FIG. 15), demonstrating a more stable metabolic phenotype in an increased oxygen environment even though the normoxic condition wasn't hypoxic (FIG. 19). In contrast, HUVEC seeded liver grafts demonstrated no significant changes in glucose consumption in normoxic versus hyperoxic conditions (FIG. 20). The decreased glucose consumption and lactate production (FIG. 16) in the increased oxygen conditions further demonstrates the delay of phenotypic dedifferentiation of primary hepatocytes from an oxidative phosphorylation metabolism that is relevant in mature hepatocytes to glucogenesis metabolism which is observed in de-differentiated and immature hepatocytes. Ammonia clearance rates were also greater in the hyperoxic conditions (FIG. 17 and FIG. 18) compared to normoxic conditions.

### Example 4: Preservation of Graft Perfusion Flow Rates when Seeding Hepatocytes into Reendothelialized Whole Liver Matrix

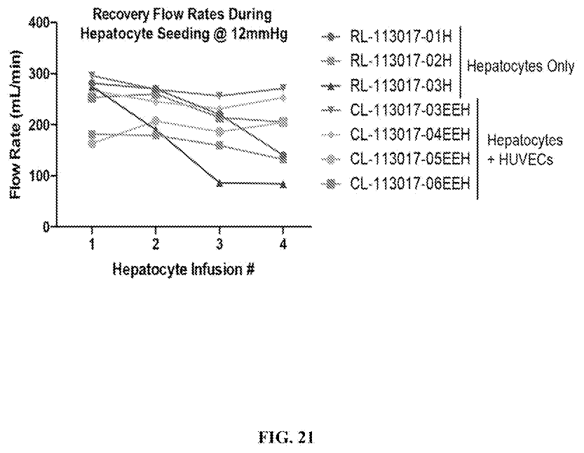

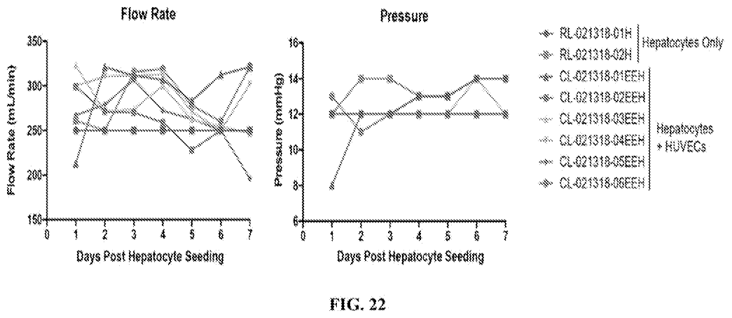

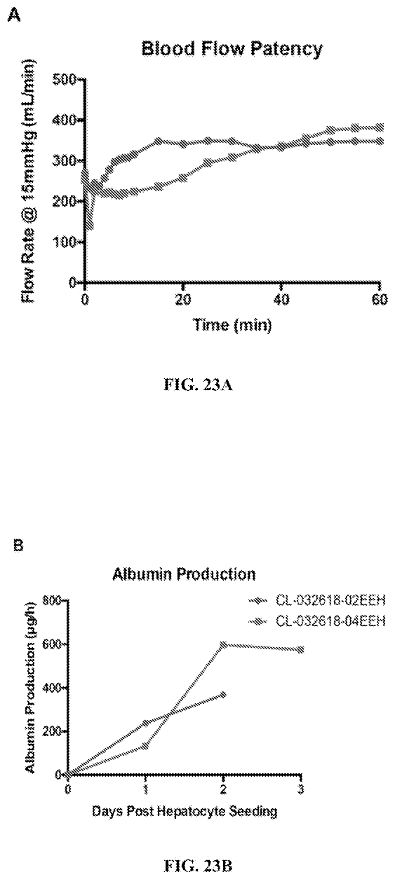

Livers were isolated from pigs, cannulated, and fully submerged in sterile water while a degassed cellular disruption media comprising sodium dodecyl sulfate was perfused through the organs until they were substantially decellularized to leave a bioengineered scaffold. The scaffolds were washed by perfusing with degassed water until the cellular disruption media was substantially removed. The washed bioengineered scaffolds were then perfused with a recellularization solution comprising HUVECs through the portal and/or hepatic veins and cultured in a bioreactor with media perfusion through the grafts at a constant pressure. The grafts were maintained in culture until glucose consumption reached a rate of at least 40 mg/h, at which point hepatocytes were infused through the hepatic vein and allowed to engraft under pressure-controlled continuous media perfusion. Recovery flow rates during hepatocyte seeding were measured and compared to organs which did not receive HUVECs prior to hepatocyte seeding. The data in FIG. 21 shows that grafts which did not receive HUVECs prior to seeding of hepatocytes experienced declining flow rates at 12 mmHG after serial hepatocyte infusions through the hepatic vein (500 million cells per infusion) compared to grafts which were first re-reendothelialized. The data in FIG. 22 shows that grafts which did not receive HUVECs prior to seeding of hepatocytes required higher perfusion pressures to maintain flow rates of 250 mL/min than grafts previously seeded with HUVECS. The data in FIG. 23A and FIG. 23B show that grafts which received HUVECs prior to seeding of hepatocytes remained competent for heparinized blood perfusion (FIG. 23A) and albumin production (FIG. 23B). 02EEH and 04EEH grafts were subjected to ex vivo heparinized blood perfusion on days 2 and 3 post hepatocyte seeding, respectively.

### Example 5: Co-Culture of Endothelial Cells and Hepatocytes

### 1. Methods

### 1.1 Decellularization of Porcine Livers

Whole livers (250-350 g) were explanted from cadaveric pigs. The portal vein, suprahepatic vein (SHV), inferior vena cava (IVC), and bile duct (BD) were cannulated and flushed with sterile saline. Cannulated livers were decellularized by peristaltic pump driven vascular perfusion with 1% Polyethylene glycol p-(1,1,3,3-tetramethylbutyl)-phenyl ether followed by 0.6% sodium dodecyl sulfate. Solution flow rates were automatically regulated by a custom perfusion control system designed to modulate solution flow rates to maintain perfusion pressures between 8-12 mmHg. Decellularized livers were subsequently disinfected with 1000 ppm peracetic acid, washed with phosphate buffered saline, and stored at 4° C. All aspects of the decellularization process were performed in an ISO 7 cleanroom facility.

### 1.2 HUVEC Cell Culture and Seeding of Decellularized Liver Scaffolds

Human umbilical vein endothelial cells (HUVECs) were cultured at 37° C. and 5% CO₂ in antibiotic-free Endothelial Cell Growth Media supplemented with 2% fetal bovine serum, ascorbic acid, hydrocortisone, FGF, VEGF, EGF, R3 IGF, heparin, and acetic acid. Cells were harvested with 0.25% trypsin-EDTA at 90-100% confluency. Decellularized porcine livers were mounted in bioreactors and perfused with antibiotic free cell culture media (37° C., 5% CO₂) for 72 h to confirm the absence of microbial contamination. HUVECs collected at passage 5-9 were infused through the SVC with a syringe (1.2×10⁸ cells in 150 mL culture media). Following one hour of static culture to allow for cell attachment within the scaffold, culture media supplemented with Penicillin and Streptomycin was perfused through the SVC at 12 mmHg. 24 hours later, a second batch of HUVECs was collected and infused through the PV in the same manner as above. Following seeding, culture media was replaced daily, and volumes were continually adjusted to ensure that glucose levels remained above 0.3 g/L within a 24 h period. Media perfusion into the scaffold was maintained at a pressure of at least 12 mmHg during culture.

### 1.3 Porcine Hepatocyte Isolation and Seeding of Bioengineered Liver (BEL) Scaffolds

Freshly harvested whole livers (400-600 g) were cannulated through the PV, SHV, and IVC, and perfused with 5 L of Hank's Balanced Salt Solution (HBSS) to remove residual blood from the organ, followed by 1 L of ice cold HTK Solution to minimize ischemic injury to organs during transportation. Livers were then perfused through the PV (500-600 mL/min) with 5 L of HBSS supplemented with 2.5 mM EGTA, allowing the first 1 L to drain to waste, and recirculating the remaining volume for 20 minutes. Livers were subsequently perfused with 2 L of solution comprised of 142 mM NaCl, 6.7 mM KCl, 10 mM HEPES, 5 mM N-acetyl-L-cysteine, and 1% Penicillin-Streptomycin. Digestion was initiated with perfusion of 4 L of L-15 media supplemented with 100 mg of Liberase™ (Sigma, 5401127001) and 5 mM CaCl₂), allowing the first 500 mL to drain to waste and recirculating the remaining volume until livers were soft with visible breakdown of the capsule (20-30 min). After digestion, 1 L of ice-cold Williams E media supplemented with 10% FBS was poured over the livers and the capsule was gently pulled apart to release the cell suspension. To eliminate any remaining undigested tissue, released cells were filtered through an 8″ kitchen strainer, followed by a series of mesh sieves 250 m, 125 m, 70 m. The filtered cell suspension was brought to a final volume of 2 L with Williams E media supplemented with 10%. Hepatocytes were enriched by low speed centrifugation (70×g, 4° C., 10 min) and washed twice in cold William's E+10% FBS. Cell viability and yield were quantified by trypan blue dye exclusion on a hemocytometer.

Following isolation, 2×10⁹ porcine hepatocytes were diluted in 2 L (1×10⁶ cells/mL) of co-culture media (Williams' E medium supplemented with 1.5% fetal bovine serum, ascorbic acid, hydrocortisone, FGF, VEGF, EGF, R3 IGF, heparin, acetic acid, human insulin, human albumin, linoleic acid, dexamethasone, human glucagon, human transferrin, and Gly-His-Lys, copper sulfate, sodium selenite, zinc sulfate, L-carnitine, L-arginine, and glycine. Hepatocytes were infused through the bile duct of reendothelialized bioengineered liver (BEL) scaffolds (typically 13-16 days following the first HUVEC seeding) with a peristaltic pump at a rate of 50 mL/min. Hepatocyte-seeded BELs were then returned to continuous media perfusion through the PV with co-culture media at a pressure of 12 mmHg.

### 1.4 Flow Cytometry

HUVEC populations were analyzed prior to seeding by anti-CD31 staining with a FITC conjugated primary antibody. Hepatocyte purity post enrichment was quantified by intracellular staining anti-albumin staining complexed with an Alexa Fluor 488 conjugated secondary antibody following fixation with 4% paraformaldehyde and permeabilization in 0.1% Triton X100. Flow cytometry analysis was performed on a BD Accuri C6 instrument and data were processed using FlowJo.

### 1.5 Histological Analysis

Tissue samples analyzed in this study were perfused with PBS and fixed with 10% Neutral Buffered Formalin. Fixed tissues were paraffin embedded, sectioned, and stained using standard histological techniques. Immunofluorescence slides were deparaffinized, rehydrated and retrieval was performed in citrate buffer, pH 6.0 in a programmable decloaker. Slides were permeabilized with PBS+0.05% Tween-20 and blocked with Sea Block. Primary antibodies used were Rabbit anti-Collagen I, Rabbit anti-Collagen IV, Mouse anti-CD31, Rabbit anti-Albumin, Rabbit anti-FAH, and Rabbit anti-LYVE1. Secondary antibodies were Goat anti-Mouse Alexa Fluor 488 and Goat anti-Rabbit Alexa Fluor 555. All antibodies were diluted in Sea Block. Slides were stained with DAPI and mounted using ProLong Antifade Mountant. H&E and immunofluorescence microscopy was performed on an Accuscope 3012 and Zeiss Axioskop 40, respectively.

### 1.6 Analysis of Cellular Metabolites and Secreted Factors During Bioreactor Culture

Media samples from bioreactors were collected daily and assayed immediately on a CEDEX BioHT analyzer to determine levels of glucose, ammonia, and lactate dehydrogenase activity in the culture media. Measured glucose concentrations were used to calculate daily consumption rates over a 24 h period prior to replenishing with fresh media. A separate aliquot of each daily media sample was stored at −80° C. and thawed at the end of each experiment for quantification of soluble vWF and albumin by ELISA.

### 1.7 Bioengineered Liver (BEL) Ammonia Clearance Kinetics and Urea Production Assays

16-20 h after seeding hepatocytes, culture media was removed from bioreactors and 2 L of co-culture media supplemented with 0.8 mM ammonium chloride. Bioreactor media perfusion was resumed, and media samples were collected in duplicate at t=0 h, 1 h, 2 h, 7 h, and 23 h. Media ammonia levels were quantified on a CEDEX BioHT, and duplicate frozen samples assayed in parallel to measure urea produced over time.

### 1.8 Acute Blood Perfusion Studies

For in vitro blood perfusion studies, each rBEL was connected to a circuit comprised of silicone tubing, a pressure transducer, and a peristaltic pump. Freshly collected heparinized porcine blood was warmed to 37° C. and activated clotting time (ACT) was measured (ITC, Hemochron Response). A solution of protamine sulfate was then gradually added to the blood to neutralize the heparin until an ACT of 170-220 was reached. 2 L of blood was introduced into the circuit and perfused through the bioengineered liver (BEL) construct at an initial flow rate of 300 mL/min, and then immediately switched to pressure-dependent PID flow control targeting a constant 12 mmHg. Flow rates and pressures were recorded over 60 minutes of blood perfusion.

In vivo acute blood studies were performed using domestic pigs weighing 80-100 kg in accordance with the Institutional Animal Care and Use Committee (IACUC) at the study center. Animals were heparinized to a target ACT of 225 sec. Recipient vessels, portal vein and IVC, were cannulated using 28F single stage venous cannula. BELs were connected to portal venous blood flow using PVC tubing and ¼″ luer-lock connectors to achieve functional end-to-side anastomoses between the graft's and recipient animal's portal veins and IVCs. Flow rates were measured using a ½″ ultrasonic flow probe connected to a controller box. Flow through the BELs were visualized via venogram. Isovue contrast was injected directly into the perfusion loop upstream of the bioengineered liver (BEL) and images were collected using an OEC 9900 Elite mobile C-arm.

### 1.9 Animal Surgeries

### 1.9.1 Heterotopic Bioengineered Liver (BEL) Implantation, Portal-Caval Shunt Surgeries, and Liver Implantation Procedure

All animal experiments were performed in accordance with the IACUC at American Preclinical Services and all experiments herein were performed in accordance with the guidelines and regulations of the committee. 28-36 kg domestic white swine were procured from a USDA-certified (class A) vendor and blood typed for type AO or A via PCR on buccal swab samples.

### 1.9.2 Anesthesia

For all animal implant procedures described below anesthesia was induced via intramuscular injection of telazol (0.5 mg/Kg) and xylazine (0.2 mg/Kg). IV access was established for fluid resuscitation of 1 L 0.9% NaCl and administration of cefazolin 1 g for surgical prophylaxis. Extended-release opiate analgesia was provided. After endotracheal, intubation, ventilation was maintained to achieve end-tidal CO₂ of 35-40 torr. Anesthesia was maintained with inhaled isoflurane 1-3%.

### 1.9.3 ICP Probe Placement

For ICP probe placement, animals were fasted 16 hr prior to the procedure, but allowed water ad lib. A scalp flap was elevated, and a 4 mm Burr hole was drilled through the frontal bone 1.5 cm lateral to midline and lcm superior to the superior orbital foramen. The dura mater was punctured bluntly, and a transdermal telemetric intracranial pressure monitor was introduced into the subdural space. The scalp flap was closed over the monitor and the animal was allowed to recover for at least five days to allow for local swelling to subside and to observe for signs of infection.

### 1.9.4 Porto-Caval Shunt

The portocaval shunt procedure was adapted from one described by Lee et al. Animals were transitioned to a soft food diet of Ensure and canned dog food three days prior to surgery and then fasted 16 hours prior to the procedure, but allowed water ad lib. Anesthesia was induced via intramuscular injection of telazol (3.5-5.5 mg/kg) and xylazine (1.5-3.5 mg/kg). IV administration of 0.9% NaCl was used as necessary for fluid resuscitation and cefazolin 1 g for surgical prophylaxis. Ketamine (˜2 mg/kg/hr), midazolam (˜0.6 mg/kg·hr) and fentanyl (˜0.004 mg/kg/hr) were used as necessary as anesthetic and analgesics. 500 mg solumedrol IV was given intravenously for immunosuppression. A bladder catheter was placed. After endotracheal, intubation, ventilation was maintained to achieve end-tidal CO₂ of 35-40 torr. Anesthesia was maintained with inhaled isoflurane 0-5%.

### 1.9.5 Ventral Laparotomy

A ventral laparotomy was performed with splenectomy followed by high hilar ligation and division. All ligaments around the liver were taken down and any aberrant vasculature was ligated and divided. A complete hepatoduodenal ligament dissection was performed and the hepatic arteries, portal vein (PV) and common bile duct were isolated. The infrahepatic inferior vena cava (IVC) was mobilized inferiorly to the level of the right renal vein taking care to preserve large local lymphatics. The caudate lobe was devascularized with aggressive parenchymal compression using a running, locking 3-0 PDS suture.

### 1.9.6 Control Group Surgery

In the control group, a direct portocaval anastomosis was performed. The animal was heparinized to a goal ACT of 170-225 sec. The IVC and the PV were partially clamped and a side-to-side anastomosis 1 cm in diameter was performed. After ensuring patency, acute ischemic liver failure was induced by ligation and division of the hepatic arteries, PV distal to the anastomosis and common bile duct. This represented time zero.

### 1.9.7 Experimental Group Surgery

In the experimental group, a bioengineered liver (BEL) graft seeded with HUVECs and porcine hepatocytes as described above was placed as opposed to a direct portocaval shunt. In order to better approximate the low portosystemic resistance of the control group, a 4 mm polytetrafluoroethylene (PTFE) shunt was anastomosed end-to-side between the recipient's PV and IVC. Patency was shown by an increase in PV flow with conduit clamping as measured by an ultrasonic perivascular flow module. Then, two 8 mm diameter, ringed PTFE prosthetic vascular grafts were anastomosed to the portal vein and infrahepatic IVC of the liver graft using running 6-0 prolene suture to bolster the anastomoses and provide additional length if needed based on the animal's individual anatomy. Preservation solution was flushed from the liver graft using 0.9% NaCl. The liver graft was coated with Tisseel aerosolized fibrin sealant to provide a physiologic pseudocapsule and permit handling and retraction as needed. The liver graft was introduced into the abdomen and placed in the auxiliary position inferior to the native liver directly anterior to the right adrenal gland. The animal was heparinized to a goal ACT of 170-225 sec. The recipient IVC and portal vein were partially clamped, and end-to-side anastomoses were performed to the tissue engineered liver graft's PTFE vascular grafts with inflow consisting of the native portal vein flowing to the graft portal vein and outflow consisting of the graft's IVC flowing into the recipient's IVC. The graft's vasculature was filled with 0.9% NaCl through its suprahepatic vena cava (SVC), and the graft was reperfused by unclamping the inflow and allowing blood flow to be visualized through the BEL's SVC prior to ligation of the bioengineered liver (BEL) SVC and unclamping of the end outflow anastomoses. PV inflow to the bioengineered liver (BEL) was measured with an ultrasonic perivascular flow module to ensure flow above 120 mL/min. If blood flow was below this value, then the PTFE shunt was partially or completely ligated to increase flow as necessary. Hemostasis was achieved with suture ligation or application of topical fibrin sealant). Whole blood transfusion of type A blood up to 1000 mL was used as needed to correct for blood loss or hemodynamic instability. Acute ischemic liver failure was induced by ligation and division of the hepatic arteries, PV distal to the anastomosis and common bile duct. This represented time zero.

### 1.9.8 Post-Hemostasis Procedures

Once the animals were vitally stable and hemostasis was achieved, an abdominal drain was placed in the surgical field and connected to bulb suction. In the case of two animals, the ultrasonic flow probe was left on the graft portal vein to monitor flow during the monitoring period. The abdomen was closed in multiple layers.

### 1.9.9 Monitoring and Resuscitation

Following surgery, a standardized monitoring and resuscitation protocol was utilized which involved hourly monitoring of vital signs, fluid output, hemodynamic parameters and ICP until death. Sedation was maintained with isoflurane 1-3% inhaled. Crystalloid resuscitation of up to 300 mL/hr and administration of phenylephrine up to 1 mcg/Kg/min titrating to a mean arterial pressure (MAP) of 50 mmHg was permitted. Five percent dextrose solution was added to crystalloid maintenance fluid to maintain blood glucose of 60-120 mg/dL. The target body temperature of 37° C. was maintained with a heating blanket. Endpoint was achieved when the animal had two consecutive hourly measurements of MAP <30 mmHg or ICP >20 mmHg and was euthanized via pentobarbital overdose.

### 1.10 Animal Imaging

Experimental animals were scanned via computer-assisted tomography (CT) of the abdomen and pelvis on a SOMATOM Definition VA44A CT scanner post-operatively. 60-90 mL of IV Optiray 350 Ioversol 350 mg/mL was administered at a contrast:saline ratio of 80:20 immediately prior to scanning. Five scans were taken every 15 seconds to ensure graft patency and successful ligation and division of all inflow vessels to the native liver in situ.

1.11 Biochemical analysis

Blood samples were obtained at time zero and every four hours following induction of acute ischemic liver failure. Blood ammonia (NH3), albumin (Alb), creatinine (Cre), blood urea nitrogen (BUN), aspartate aminotransferase (AST), alanine aminotransferase (ALT), total bilirubin (tBil), lactate dehydrogenase (LDH) and alkaline phosphatase (ALP) levels were measured. Blood glucose (Glu), hemoglobin concentration, electrolytes, pH levels, blood gasses, ACT and prothrombin time (PT/INR) were determined using an automatic point-of-care biochemical analyzer.

### 2. Results

2.1 Characterization of Bioengineered Liver (BEL) Constructs Seeded with Primary Endothelial Cells and Hepatocytes

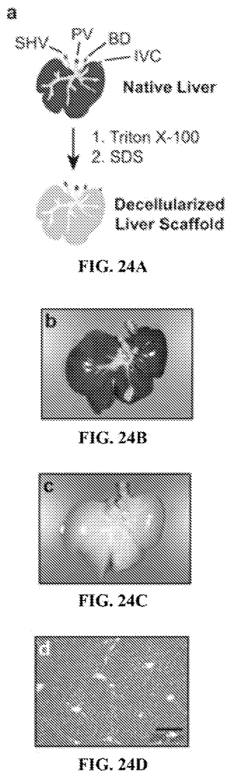

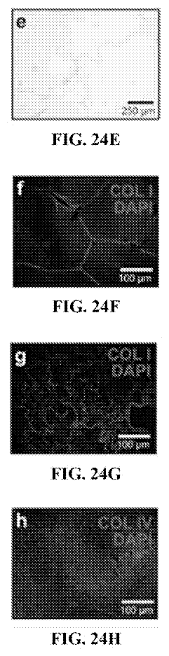

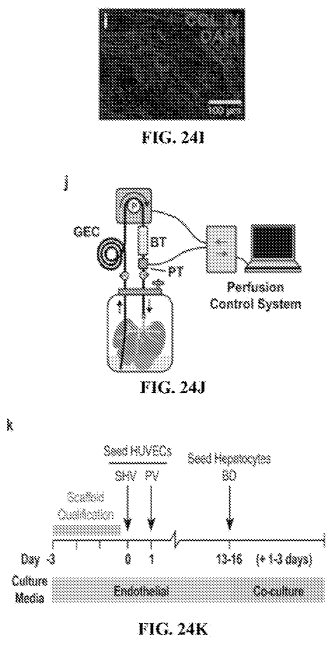

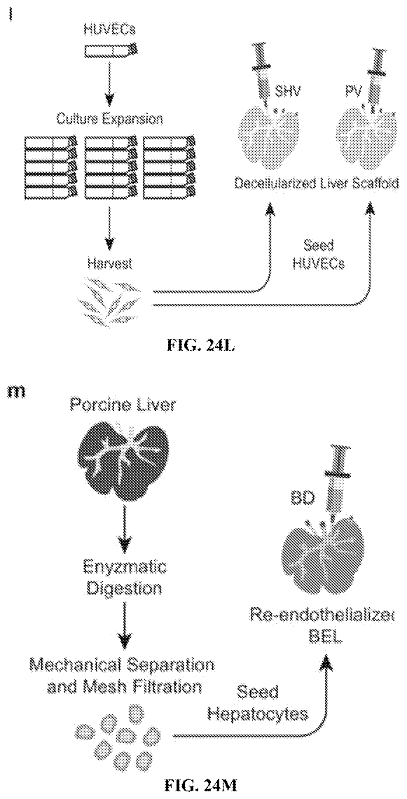

Freshly explanted porcine livers were decellularized by sequential perfusion with Triton X-100 and SDS detergent solutions as described previously to generate the acellular scaffolds shown (FIG. 24A, FIG. 24B, and FIG. 24C). Histological staining showed complete removal of cellular material from the decellularized scaffold (FIG. 24D, FIG. 24E) and retention of the extracellular matrix (ECM) proteins collagen I and collagen IV (FIG. 24F, FIG. 24G, FIG. 24H, and FIG. 24I). Decellularized livers were mounted in custom bioreactors (FIG. 24J) and perfused with antibiotic-free endothelial culture media for 72 h to confirm the sterility of the scaffold (FIG. 24K).

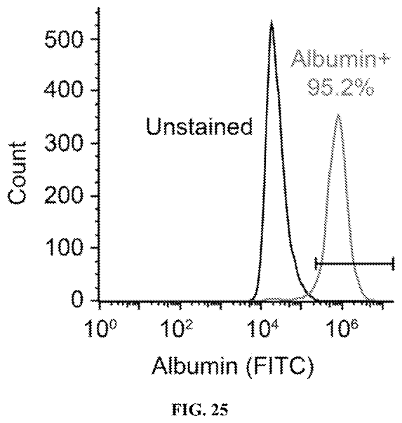

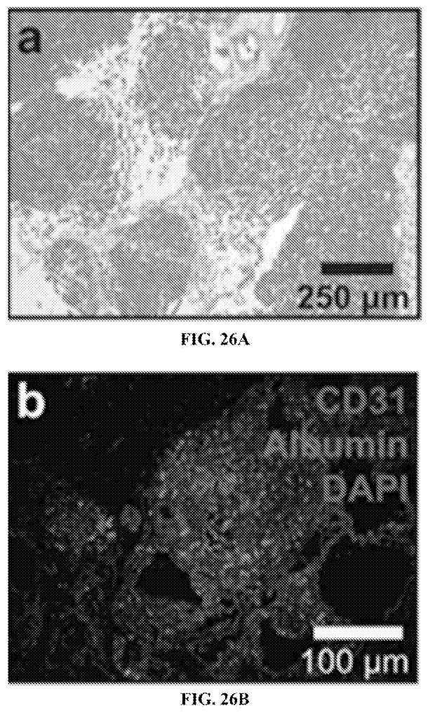

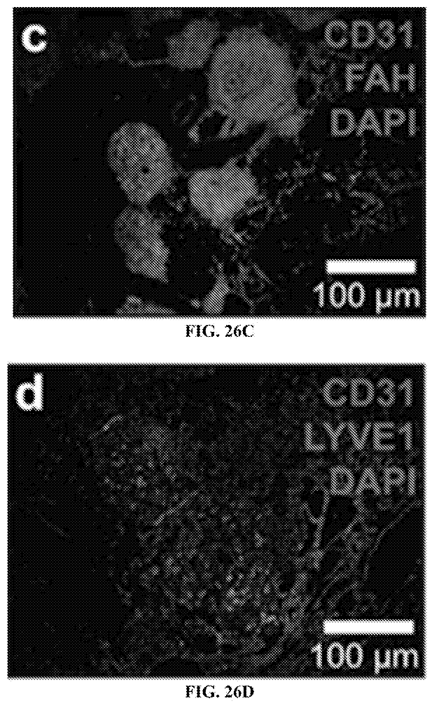

Human umbilical vein endothelial cells (HUVECs) were expanded, analyzed for CD31 expression by flow cytometry at the time of harvest (FIG. 25), and seeded into decellularized scaffolds through the suprahepatic vein (SVC), followed by the portal vein (PV) 24 h later (FIG. 24K and FIG. 24I). It was previously reported that daily glucose consumption rates (GCR) provide a robust, non-invasive metric for monitoring cell proliferation in HUVEC-seeded bioengineered liver (BEL) constructs and is predictive of successful perfusion outcomes in vivo. BELs were cultured until a minimum GCR of 50 mg/h was observed (typically 13-16 days following HUVEC seeding) prior to infusing hepatocytes into the scaffold (FIG. 24K and FIG. 24L).

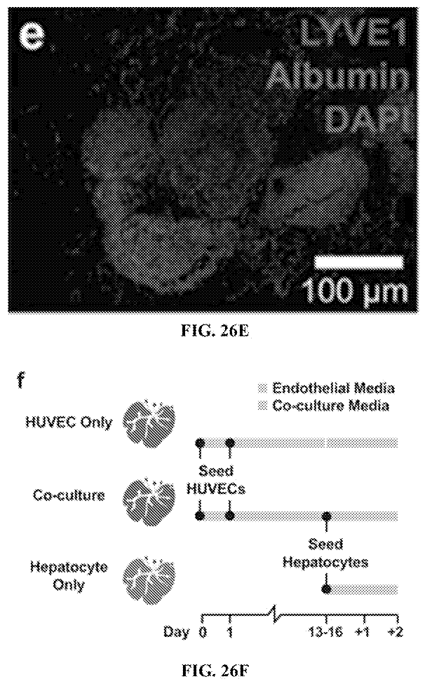

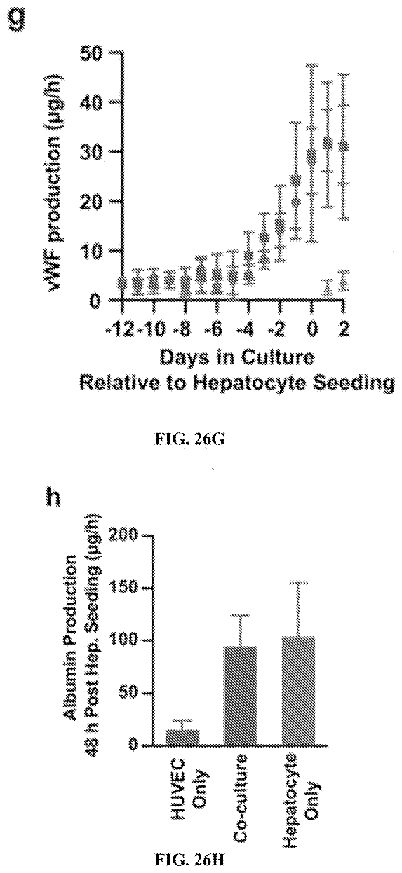

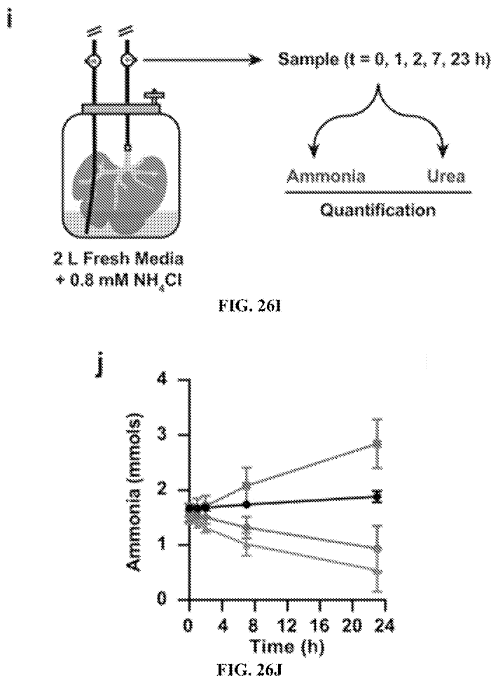

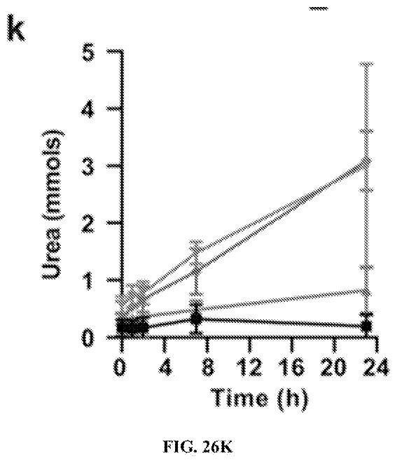

Primary hepatocytes were isolated from freshly explanted porcine livers and selected by differential sedimentation through multiple low speed centrifugation and washing steps to achieve typical hepatocyte enrichment of 85-95% as measured by albumin-positive flow cytometry. Hepatocytes were seeded through the bile duct and cultured for an additional 48 h under continuous PV perfusion with media formulated for the simultaneous culture of endothelial cell and hepatocytes (referred to herein as co-culture media; see Methods). Hematoxylin and eosin (H&E), as well as immunofluorescence staining for hepatocyte-specific markers (albumin and fumarylacetoacetate [FAH]) 48 h post hepatocyte seeding revealed a clustered distribution of hepatocytes throughout the scaffold parenchyma (FIG. 26A, FIG. 26B, and FIG. 26C). HUVECs exhibited multiple endothelial cell phenotypes over the course of perfusion culture. Similar to native liver tissue, endothelial cells localized within large vessels expressed high levels of CD31, while endothelial cells localized within parenchymal capillaries had increased expression of the liver sinusoidal endothelial cell (LSEC) marker LYVE1 and reduced expression of CD31 (FIG. 26B, FIG. 26C, and FIG. 26D). Notably, LYVE1 expressing endothelial cells were observed in hepatocyte dense regions of parenchymal tissue (FIG. 26E).

To further characterize the in vitro function of BELs over the course of bioreactor culture, levels of cell-derived soluble factors were quantified in culture media samples from scaffolds seeded with both HUVECs and hepatocytes (Co-culture), as well as scaffolds that received only one cell type (HUVEC-only and Hepatocyte-only) (FIG. 26F). BELs seeded with HUVECs (HUVEC-only and Co-culture) exhibited increasing production of the endothelial cell-derived von Willebrand factor (vWF) over time (FIG. 26G). Importantly, vWF production in Co-culture BELs following hepatocyte seeding was similar to levels observed in HUVEC-only bioengineered liver (BEL) controls, suggesting that the addition of hepatocytes to the scaffold did not compromise endothelial cell viability and function. Albumin levels were quantified in media samples from Co-culture and Hepatocyte-only BELs following hepatocyte seeding in parallel with matched HUVEC only controls, and no appreciable difference in albumin production between Co-culture and Hepatocyte-only BELs was observed, suggesting that hepatocyte function was similarly not impacted by endothelial cells in the scaffold. (FIG. 26H). To further assess key metabolic functions of hepatocytes in bioengineered liver (BEL) scaffolds, ammonia detoxification and urea production assays were adapted to bioreactor scale culture based from prior 2D culture and hepatocyte spheroid studies. In brief, BELs were challenged with fresh co-culture media supplemented with 0.8 mM ammonium chloride 16-20 h following hepatocyte seeding. As expected, HUVEC-only BELs showed a steady increase in ammonia levels over time due to ongoing cellular metabolism. In contrast, Hepatocyte-only and Co-culture BELs both showed significant ammonia clearance with similar kinetics although Hepatocyte-only BELs showed slightly more ammonia clearance (FIG. 26J). Hepatocyte-only and Co-culture BELs showed significant increases of urea, while HUVEC-only BELs showed only a slight increase in urea compared to a bare graft (FIG. 26K). These assays confirm the ability of hepatocytes to retain their function in high density cultures in a decellularized liver scaffold. In addition, the data shows that endothelial cells cultured in the scaffold did not significantly impede the functionality of hepatocytes in the scaffold.

### 2.2 Acute Blood Perfusion Studies to Assess Vascular Patency of BELs

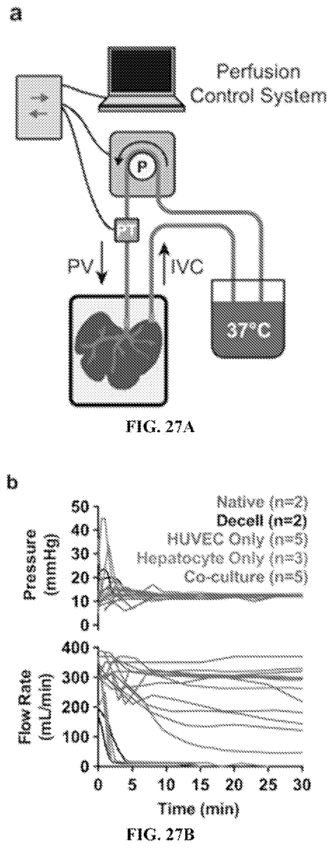

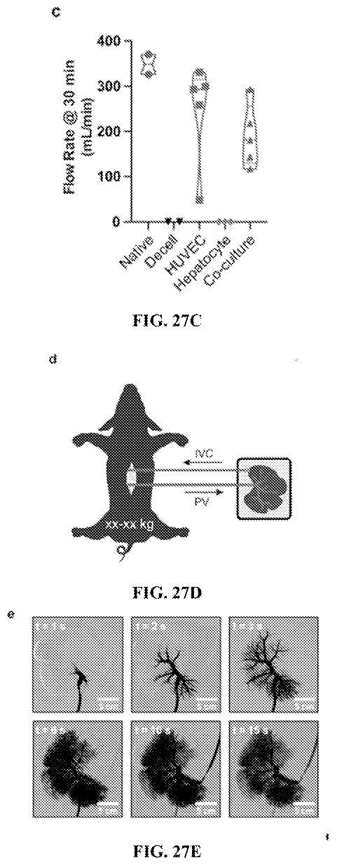

An ability to sustain blood perfusion in vivo is essential to the successful development of any bioengineered organ that is functionally reliant on an extensive vascular network. To determine the ability of BELs in this current study to sustain acute blood flow, an ex vivo blood perfusion circuit was created to recirculate porcine blood through BELs (seeded and cultured as shown in FIG. 26F) at a regulated target pressure of 12 mmHg while monitoring flow rates over 30 minutes (FIG. 27A). To mimic the initial graft reperfusion rates experienced in a surgical liver transplant model, initial blood flow through the BELs was set to 350 mL/min to fully inflate the graft, and flow rates were subsequently allowed to automatically correct to target a perfusion pressure of 12 mmHg. Additionally, prior to bioengineered liver (BEL) perfusion, the activated clotting time (ACT) of pre-heparinized porcine blood (ACT >1500) was titrated to a slightly elevated physiologically relevant range of 170-230 (just high enough to inhibit clotting within silicone tubing) through the addition of protamine sulfate to allow clotting to occur. To establish benchmarks for optimal perfusion as well as scaffold thrombosis in this system, freshly explanted porcine livers (n=2) and unseeded decellularized liver scaffolds (n=2) were subjected to continuous perfusion in the blood circuit. As expected, native organs exhibited relatively stable flow rates in excess of 300 mL/min by the end of 30 minutes, whereas decellularized scaffolds thrombosed within 5 minutes (FIG. 27B and FIG. 27C). Four out of five HUVEC-only BELs exhibited relatively stable flow rates (250-350 mL/min) over 30 minutes (similar to freshly explanted livers), while one lost flow gradually over the course of perfusion (FIG. 27B and FIG. 27C). Similar to the decellularized scaffolds, Hepatocyte-only BELs (n=3) experienced a rapid loss in flow within the first five minutes, (FIG. 27B and FIG. 27C), reaffirming the importance of endothelial cells for continuous blood perfusion through the scaffold. Co-culture BELs (n=5) generally exhibited more intermediate flow rate profiles similar to those observed for native livers with slightly lower flow rates (FIG. 27B and FIG. 27C), presumably due to increased impedance within the scaffold from the hepatocyte seeding. Nevertheless, all Co-culture BELs still maintained significant blood flow (118-293 mL/min) after 30 minutes of continuous perfusion at physiologic pressures.

To further characterize the vascular blood flow through liver scaffolds seeded with both HUVECs and hepatocytes, a porcine ex vivo blood perfusion model was employed whereby the PV and IVC of an anesthetized pig (80-100 kgs) were cannulated and connected with tubing to establish a perfusion circuit through the Co-culture grafts under physiologic venous flow (FIG. 27D). Real-time angiography performed after 30 minutes of continuous perfusion revealed flow through major vessels and capillary beds in the majority of the scaffold (FIG. 27E).

### 2.3 Implantation of Co-Culture BELs in a Large Animal Model

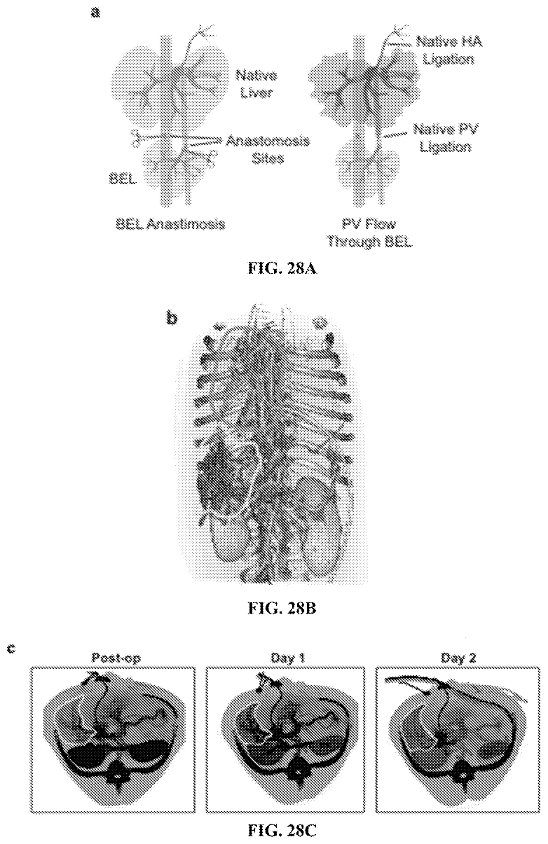

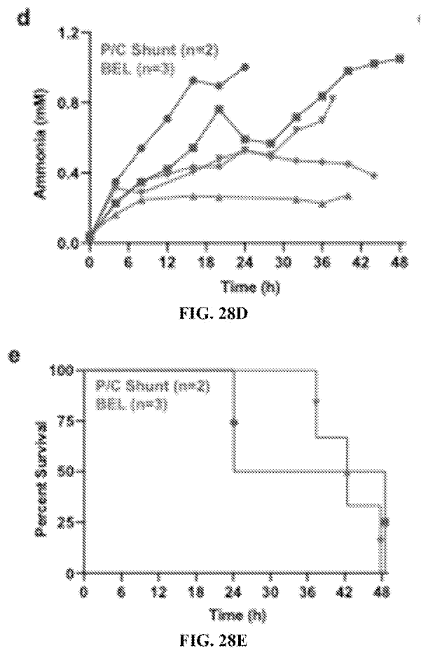

To assess the ability of co-culture BELs to sustain 48 h of continuous physiologic perfusion and assess hepatocyte function in vivo, a porcine heterotopic acute liver transplant model was employed. BELs were situated in a heterotopic position inferior to the native liver and end-to-side anastomoses were performed between the bioengineered liver (BEL) PV and IVC to the native PV and IVC, respectively (FIG. 28A). Total portal vein flow was diverted into the bioengineered liver (BEL) by ligating the native PV between the bioengineered liver (BEL) and native liver. The implanted bioengineered liver (BEL) (260±40.8 g) was ˜30% the native liver size resulting in a large mismatch. In one animal, the size mismatch contributed to the development of portal hypertension over time which necessitated the use of a portocaval shunt in the model to off load ˜10% of total blood flow and reduce portal hypertension. To reduce portal hypertension but ensure majority flow through the implanted bioengineered liver (BEL) a small portal/caval shunt utilizing a 4 mm polytetrafluoroethylene (PTFE) shunt was anastomosed end-to-side between the recipient's PV and IVC. Blood flow through the portal/caval shunt was measured at between 20 and 50 mL/min, or ˜10% of the flow through the BEL.

Following bioengineered liver (BEL) perfusion, the native liver was further isolated from arterial perfusion by ligating the hepatic arterial branches and prevent retrograde flow from the IVC was limited by applying compressive sutures to the native caudate lobe. Flow through the bioengineered liver (BEL) the first few hours after perfusion was (120-410 mL/min) as measured through by transonic probes prior to closing. Computed tomography (CT) was utilized post-operatively to confirm bioengineered liver (BEL) perfusion and loss of flow to the native liver (FIG. 28B and FIG. 28C). Follow up CT imaging at Day 1 and Day 2 confirmed that bioengineered liver (BEL) perfusion was sustained throughout the remaining duration of the acute implant studies (FIG. 28C).

### 2.4 Functional Characterization of BELs In Vivo

In addition to showing sustained perfusion of a hepatocyte/endothelial bioengineered liver (BEL) in vivo, the survival and functionality of hepatocytes was assessed. Previous studies have correlated the relationship between intracranial pressure (ICP) and blood ammonia levels during acute liver failure. Therefore, an intercranial probe was implanted 5 days prior to surgery to allow regular monitoring of the intercranial pressure (ICP) (at least once every hour). Termination of the study was performed when an animal experienced; 1) ICP of 20 mmHg or greater for 2 hours, 2) mean arterial pressure (MAP) was 30 or less for 2 hours, or 3) 48 hours of survival was achieved. The control group that experienced the implantation of a Portal Vein/IVC shunt resulted in termination at 24 and 48 h (FIG. 28E). The 24-h survivor was terminated due to a MAP less than 30, at which point the ICP was 17. The 48-h survivor had a MAP of 42 and ICP of 16 at the time of termination. Blood ammonia levels in the control animals climbed quickly with levels surpassing 0.5 mM within 15 hours of surgery (FIG. 28D).

In contrast, the experimental group receiving bioengineered liver (BEL) implants were terminated at 37 hours, 42 hours, and 48 hours (FIG. 28E). The 37-h survivor showed an ICP greater than 20 mmHg with a MAP of 37 mmHg. A 42-h survivor had a low MAP secondary to respiratory failure with an ICP of 11. The 48-h survivor reached the end of study and had a MAP of 38 and ICP of 17. The bioengineered liver (BEL) group also showed an increase in blood ammonia levels to approximately 0.5 mM, but leveled off or decreased, except for one graft that lost flow late (37-h survivor) that resulted in a rapid increase in ammonia and ICP that prompted termination at 37 hours (FIG. 28D). The ammonia data is suggestive of hepatocyte function and is consistent with ex vivo ammonia clearance data. However, interpretation of the results of the heterotopic implant model may have been confounded by collateral blood flow that was not isolated or by factors secreted by the isolated ischemic liver into the peritoneal cavity. Histological examination and immunostaining of explanted BELs for albumin and FAH (FIG. 28E and FIG. 28F) and CD-31 showed viable porcine hepatocytes and HUVECs throughout the explanted bioengineered liver (BEL) suggesting that seeded cells remained viable and phenotypically stable throughout the duration of in vivo perfusion.

### Example 6: Recellularizing a Bioengineered Scaffold while in Solution

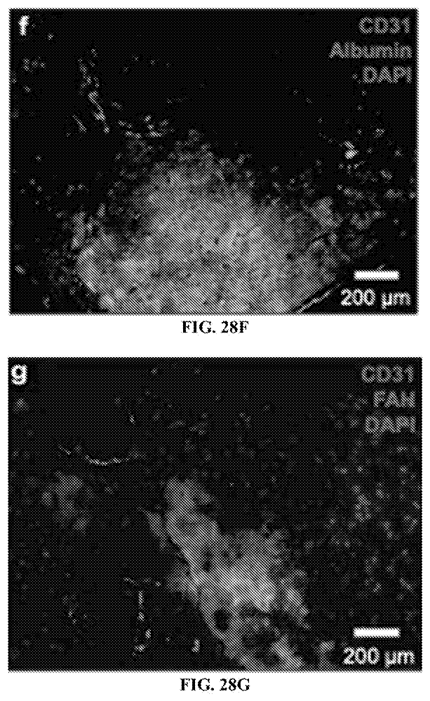

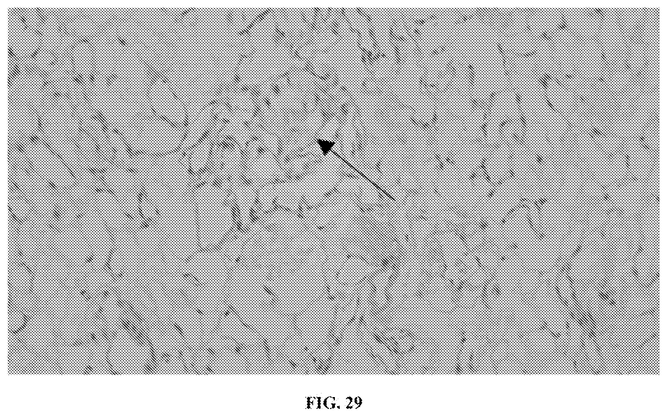

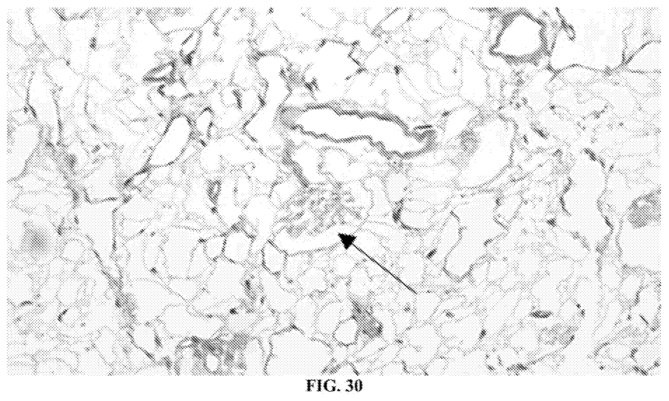

Decellularized whole kidneys were seeded with endothelial cells while remaining in solution to provide a reduced gravity or buoyance environment to enhance cellular seeding through the vasculature compared to suspending the kidney in air during the initial seeding. Histological examination demonstrated enhanced engraftment and distribution of organs seeded while in solution compared to suspension seeding. FIG. 29 depicts H&E staining of a decellularized kidney seeded with HUVECs via perfusion while in solution. The arrow points to the glomerulus. FIG. 30 depicts H&E staining of a decellularized kidney seeded with HUVECs via perfusion while suspended in air. The arrow points to the glomerulus.

### Example 7: Bile Duct Seeding of Hepatocytes to a Revascularized Liver

### Ammonia Clearance Method:

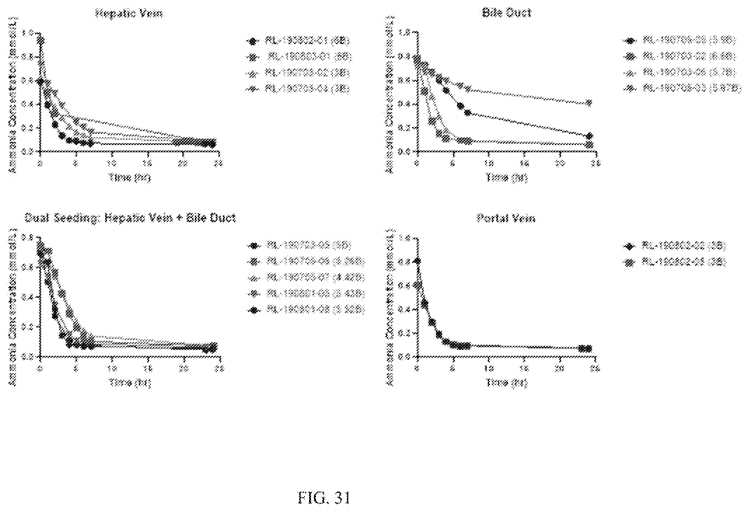

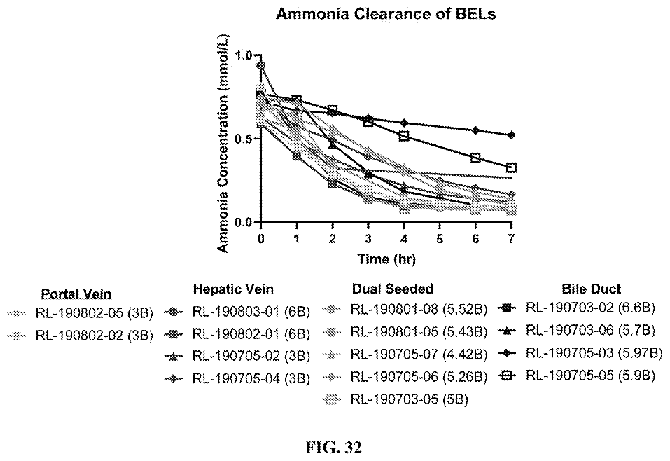

BEL ammonia clearance kinetics and urea production assays. 16-20 h after seeding hepatocytes in revascularized liver grafts, culture media was removed from bioreactors and 2 L of bi-culture media supplemented with 0.8 mM ammonium chloride. Bioreactor media perfusion was resumed, and media samples were collected in duplicate at t=0 h, 1 h, 2 h, 7 h, and 23 h. Media ammonia levels were quantified on a CEDEX BioHT, and duplicate frozen samples assayed in parallel to measure urea produced over time. Results are shown in FIG. 31 and FIG. 32. FIG. 31 depicts ammonia clearance rates of revascularized livers seeded with hepatocytes via the hepatic vein, bile duct, dual seeding or portal vein. The actual number of seeded hepatocytes is defined in parentheses for each liver. FIG. 32 depicts ammonia clearance rates of revascularized livers seeded with hepatocytes via the hepatic vein, bile duct, dual seeding (hepatic and bile duct) or portal vein. The actual number of seeded hepatocytes is defined in parentheses for each liver.

Grafts seeded via the portal, hepatic or in combination with the bile duct demonstrated increased ammonia clearance rates compared to bile duct seeded hepatocytes. In additional to ammonia clearance rates, the ability of the grafts to remain patent in a blood loop was evaluated. The data demonstrates that while the hepatic and portal vein seeded grafts demonstrated the greatest ammonia clearance rates, the hepatic vein seeded grafts demonstrated superior patency compared to the portal vein seeded livers. In addition, grafts seeded through bile duct demonstrated good patency.

The importance of this data is the demonstration that seeding through the bile duct or the hepatic vein results in a liver graft that has function and capable of blood perfusion. In contract, livers seeded via the portal vein demonstrate good function but fail to achieve adequate blood perfusion at a physiological pressure.

### Blood Loop Method:

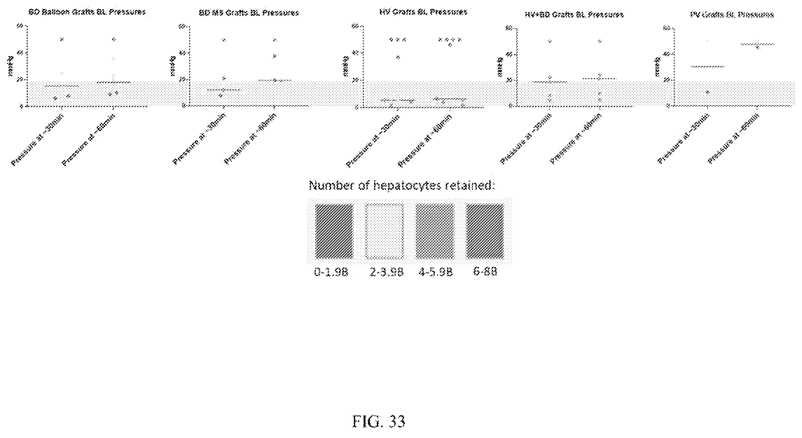

For in vitro blood perfusion assays, each liver graft was connected to a circuit comprised of silicone tubing, a pressure transducer, and a peristaltic pump. Freshly collected heparinized porcine blood was warmed to 37° C. and activated clotting time (ACT) was measured (ITC, Hemochron Response). A solution of protamine sulfate was then gradually titrated into the blood to neutralize the heparin until an ACT of 170-220 was reached. 3 L of blood was introduced into the circuit and perfused through the bioengineered liver (BEL) construct at constant flow rate of 350 mL/min. Portal pressures were recorded over 60 minutes of blood perfusion and are shown in FIG. 33.

Tri-Culture Grafts with HV-Seeded Hepatocytes:

Grafts were seeded with HUVECS (300-400 million) via the HV & PV and were then cultured for 10 days. Cholangiocytes (200-270 million) were then seeded via the bile duct and grafts were cultured 4-5 more days. Hepatocyte seeding took place after the grafts had been in culture 14-15 total days. 3-4 billion hepatocytes were seeded using the balloon method via the HV. Grafts were cultured 2 more days before fixation and processing for histology/immunofluorescent staining.

Sections taken from each liver lobe were stained with the following antibodies, which are specific for cholangiocytes in the liver: Epithelial cell adhesion molecule (EpCAM), Cytokeratin 7 (CK7), Cytokeratin 19 (CK19), and Pan-Cytokeratin (PanCK). An antibody against Albumin (Alb) was used to identify hepatocytes and an antibody against Cluster of differentiation 31 (CD31) marked endothelial cells.

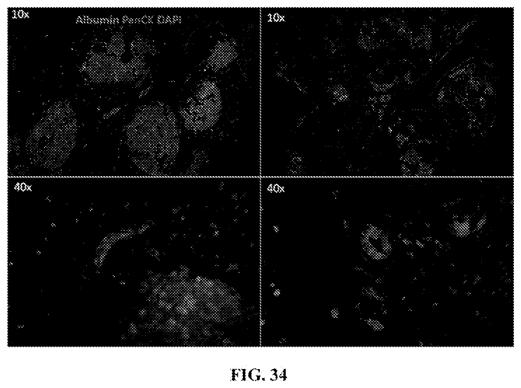

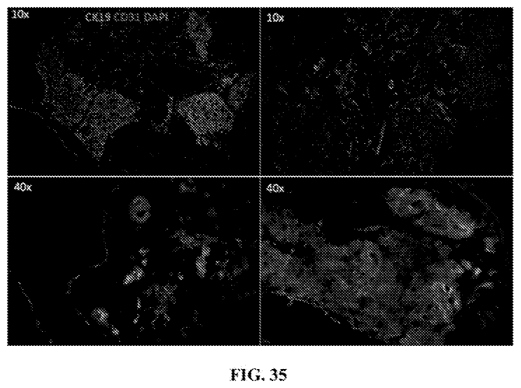

Cholangiocytes positive for EpCAM, CK7, CK19, and/or PanCK were found in all lobes examined as shown in FIG. 34 and FIG. 35. Cells were unevenly distributed and appeared in three general patterns: multicellular patches ranging in size from hundreds to thousands of cells, smaller elongated patches without an obvious lumen, and rare tubule-like structures with a distinct luminal space. Cells in large patches or in tubule-like structures tended to show strong EpCAM localization at cell membranes. Cells found in tubule-like structure additionally showed EpCAM and cytokeratin polarization. Cells in elongated patches generally showed strong cytokeratin staining, however EpCAM was not often localized to cell membranes. In all lobes, cholangiocytes appeared in regions adjacent to but distinct from hepatocytes and endothelial cells. Hepatocytes appeared in medium-to-large patches, likely filling up lobules. Cholangiocytes were often present in areas between lobules, next to but separate from reendothelialized vessels.

Tri-Culture Grafts with BD-Seeded Hepatocytes:

Grafts were seeded with HUVECS (300 million) via the HV & PV and were cultured for 13 days. Cholangiocytes (165 million) were then seeded via the bile duct and grafts were cultured 6 more days. Hepatocyte seeding took place after the grafts had been in culture 18 total days. 2 billion hepatocytes were seeded using the balloon method via the BD. Grafts were cultured 2 more days and went through an ammonia clearance test before fixation and processing for histology/immunofluorescent staining.

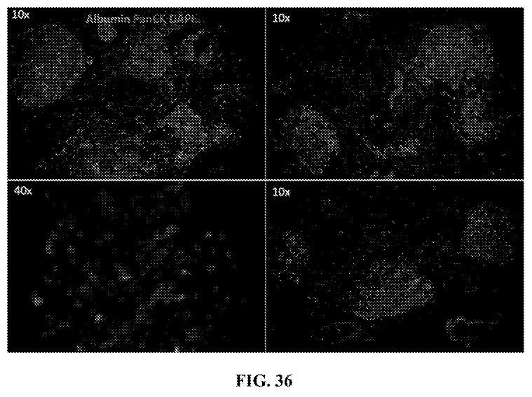

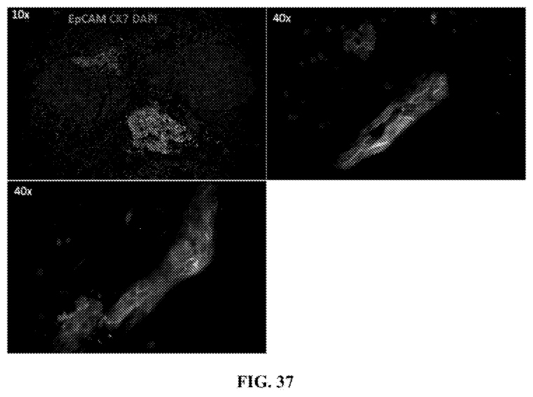

Sections from liver lobes were stained and analyzed as described for the HV seeded hepatocytes. Cholangiocyte distribution and results for EpCAM and cytokeratin staining were very similar to what had been seen previously with HV-seeded-hepatocyte triculture grafts as shown in FIG. 36 and FIG. 37. Hepatocytes were found in small-to-large patches, distributed unevenly, with some found in lobules and many in non-lobular spaces including presumed biliary structures. Examples of cell patches with intermixed hepatocytes and cholangiocytes were found in all lobes. Endothelial cells were generally present adjacent to but distinct from patches of hepatocytes/cholangiocytes.

### Example 8: Induction of Pluripotency, Plating and Maintenance of Pluripotent Stem Cells

### Inducing Pluripotency

Fibroblast cells are obtained from a donor, and pluripotency induced using a single polycistronic lentiviral vector containing the four-factor stem cell cassette (STEMCCA). Co-transfection is performed with the plasmid Gag-Pol and the helper plasmid encoding VSVG into HEK293T cells to produce viruses. After infection cells are cultured in ES medium starting from 1 day post-infection and treated with 0.9 mM VPA for 2 weeks. A Cre-recombinase-mediated vector excision can then optionally be performed to remove the four factors from the genome of the cells. hiPSCs are harvested with 0.05% trypsin/EDTA or another suitable passaging reagent, re-suspended in PBS (1×10⁶ cells) and transfected by electroporation with pCAG-Cre-EGFP. Cre-recombinase eGFP-expressing cells are selected from a single-cell suspension by FACS sorting 72 hours after electroporation. Selected cells are re-plated at low density in ES medium containing the ROCK inhibitor Y-27632. Cells are tested for pluripotency by qPCR and immunochemistry detection of Oct-4, Klf-4, C-Myc, Tra-1-60, and Nanog.

### Plating and Maintaining Pluripotent Stem Cells

For this protocol human embryonic stem cells or induced pluripotent stem cells can be used as the source of stem cell. Cells are thawed from liquid nitrogen storage and plated on either E-Cad-FD (StemAdhere), Matrigel, Vitronectin, or fibronectin-coated tissue plates or flasks in a suitable stem cell medium (e.g. mTesR1, Essential 8, or Essential 8 Flex). Cells are grown until they reach approximately 70% confluency and are passaged using Accutase, TrypLE, dispase, or another suitable passaging reagent. After cells have been maintained in culture for at least two passages they are tested for pluripotency markers Tra-1-60, Oct-4, Nanog, Klf4, c-Myc, and Sox2 using qPCR or FACS. Cells are examined using light microscopy to ensure a starting population of high quality homogeneous cells to improve the success of differentiation. Cells must be no more than 50% of the surface of the tissue culture dish and exhibit minimal evidence of differentiation. The culture media is removed from the dish, and a suitable volume of DPBS added, swirled and removed to rinse the cells. A suitable volume of DPBS with 0.02% EDTA is then added and the dish allowed to incubate for two minutes at room temperature. As soon as the cells begin to detach the DPBS with EDTA is removed and the plate flooded with a suitable volume of pluripotent stem cell medium. For more adhesive basement membrane matrix substrates the cells can be detached using a harsher passaging reagent such as Accutase or TrypLE. The cells are dissociated into small clusters containing 3-6 cells by pipetting. The cells are collected by centrifuging at 200×g for 5 minutes, the supernatant removed, and the pellet resuspended in stem cell culture medium. The cell suspension is transferred to a number of 35 mm wells of a 6-well tissue culture plate, which has previously been coated with matrigel (matrigel is thawed on ice at 4° C. overnight and added to ice cold DMEM before being plated onto the plate and allowed to incubate overnight at 37° C. in a 5% CO₂ incubator). The cells are cultured on the matrigel coated plate overnight at 37° C., 4% 0, and 5% CO₂. The number of cells that produces an optimal differentiation varies from cell line to cell line and should be determined empirically; however, in many cases a 100 mm tissue culture dish whose surface is ˜50% covered with stem cell colonies is sufficient to seed 2-3×35 mm wells. After overnight culture cells should form a monolayer that covers 80-100% of the surface of the dish. The cell density at the initiation of differentiation can have a dramatic impact on differentiation efficiency and may need to be determined empirically for each cell line. In addition, if the cells are left for greater than 24 hrs before initiation of differentiation it commonly has a negative impact on the efficiency of differentiation.

### Example 9: Differentiation of Stem-Cell Derived Liver Cells

### Differentiation of Stem-Cell Derived Hepatocytes

Cells are plated and maintained in a pluripotent state using a method such as that described in example 4. On each of the first two days after plating the cells the culture medium is replaced with RPMI medium which has been pre-warmed to 37° C. supplemented with 2% B27 (without insulin), 100 ng/ml Activin A, 10 ng/ml BMP4, and 20 ng/ml FGF2 and culture with daily medium changes for 2-days at 37° C. in ambient O₂/5% CO₂. 1 μM of the PI-3 kinase inhibitor LY 294002 can be added as an alternative to B27. On each of differentiation days 3-5 the culture medium is changed to RPMI/2% B27 (without Insulin) containing 100 ng/ml Activin A with daily medium changes, continuing to culture the cells at 37° C., ambient O₂/5% CO₂. At the end of this stage of the differentiation it is crucial that >90% of cells express proteins that are characteristic of the anterior definitive endoderm including CXCR4, FOXA2, SOX17, and GATA4. In addition, the presence of proteins associated with pluripotent cells such as OCT4 should be minimal if detected at all. From differentiation days 6-10 hepatic differentiation is induced by changing the medium to RPMI/2% B27 (with Insulin) supplemented with 20 ng/ml BMP4 and 10 ng/ml FGF2. After 5 days of culture the cells should form a continuous monolayer and 80-90% of cells should express HNF4a and the levels of GATA4 and SOX17 should have declined. On differentiation days 11-15 the hepatic progenitor cells are cultured for 5 days in RPMI/2% B27 (with Insulin) supplemented with 20 ng/ml hepatocyte growth factor (HGF) with daily medium changes. In addition to HNF4a, 80-90% of the cells should now express AFP and lipid droplets are commonly observed within the cytoplasm of the cells. Finally, several mRNAs which are enriched in fetal hepatocytes can be detected including Fibrinogen alpha chain (FGA), Fibrinogen gamma chain (FGG), Transferrin (TF), and Angiotensinogen (AGT). On differentiation Days 16-20 the medium is replaced with Clonetics® Hepatocyte Culture Medium (HCM™) containing the supplied ‘Singlequots’, but omitting the EGF from the HCM™ ‘bullet’ kit. The medium is supplemented with 20 ng/ml of Oncostatin-M. Continue to culture the cells for at least 5-days with daily medium changes at 37° C., ambient O₂/5% CO₂. By day 20 of the differentiation protocol the cells should display a morphology that resembles primary hepatocytes with a distinct cuboidal morphology and a large cytoplasmic to nuclear ratio. In addition, 70-90% of cells should express Albumin and the Asialoglycoprotein receptor (ASGPR) and the level of Albumin secreted into the culture media can approach >70% of that found for primary hepatocytes.

### Determination of Liver Cell Identity

Liver cell identity can be determined through qPCR analysis of extracted RNA, or immunohistochemistry or FACS analysis of proteins. The early markers of hepatocyte differentiation include Mixl1 (indicating the primitive streak), Sox17 (indicating the definitive endoderm), Hnf4a (indicating the foregut), and Tbx3 (indicating the liver bud). Mature markers of hepatocytes and other liver cells that are tested for include; albumin, fibrinogen, cytochrome P450 3A4 (CYP3A4), asialoglycoprotein receptor 1 (ASGR1), alpha-1 antitrypsin (AAT), carbamoyl phosphate synthetase 1 (CPS1), fumarylacetoacetate hydrolase (FAH), and homogentisate 1,2-dioxygenase (HGD). Cells that have not completely differentiated will instead show expression of pluripotency markers Tra-1-60, Oct-4, Nanog, Klf4, c-Myc, and Sox2.

### Example 10: Differentiation of Stem-Cell Derived Heart Cells

### Differentiation of Cardiomyocytes

Cells are plated and maintained in a pluripotent state using a method such as that described in example 4. To induce differentiation, stem cells are first dispersed into small clumps using collagenase-IV and are then cultivated in a suspension for 10 days as embryoid bodies and plated on 0.1% gelatin-coated culture dishes. Cardiomyocytes are separated by FACS analysis to select for the expression of sarcomeric-α-actinin, cTnI, and cTnT to confirm the identity of the differentiated cells as cardiomyocytes. RT-PCR is also performed to detect increased levels of NKX2-5, MYH-6, MYH-7, MLC-2v, TNNI, andMYL7, while undifferentiated cells will express Tra-1-60, Oct-4, Nanog, Klf4, c-Myc, and Sox2.

### Example 11: Differentiation of Kidney Cells

### Differentiation of Stem-Cell Derived Podocyte Cells

Cells are plated and maintained in a pluripotent state using a method such as that described in example 4. Cells can be iPSCs derived from kidney cells or from another cell type which has been reverted to a naïve ground state. Pluripotent cell colonies are mechanically cut into small pieces approximately the same size and are cultured in ultra-low cluster 6-well plates for three days in a differentiation medium consisting of DMEM-F12 with 2.5% FBS, 100 μM nonessential amino acids, 100 μM beta mercaptoethanol with the addition of 10 ng/ml of activin A, 15 ng/ml of BMP7, and 0.1 μM retinoic acid. The cells are transferred into 0.1% gelatin pre-coated 10 cm tissue culture dishes for another 7-8 days in the same medium before serial sub-passaging for characterization and integration assays. At 10 days of differentiation the iPS podocytes are fixed in 2.5% glutaraldehyde in cacodylate buffer and processed for SEM visualization. For the long-term maintenance of iPSC-derived podocytes, after 10 days of directed differentiation the iPSC-derived podocytes are grown in DMEM-F12 media without the addition of 10 ng/ml of activin A, 15 ng/ml of BMP7, and 0.1 μM retinoic acid where they are able to maintain the morphological characteristics and functional capacity.

### Determination of Podocyte Identity

Determination of podocyte identity is determined by immunocytochemistry. Differentiated iPS podocytes and human podocytes are seeded on chamber slides in 10% FBS medium and are serum starved overnight, then fixed in 4% paraformaldehyde for 10 min, permeabilized with 0.1% Triton X-100/PBS for 10 min and incubated in blocking solution for 30 min. The cells are incubated with anti-nephrin, anti-synaptodin, anti-Pax-2, and anti-podocin, and anti-WT1 at dilutions from 1:20 to 1:400 in blocking solution overnight at 4° C. and incubated with secondary antibodies in PBS for 1 hr. Sections are counterstained with DAPI and then mounted with fluorescent mounting medium and analyzed with a Provis AX70 or Nikon C1 confocal fluorescent microscope. Successfully differentiated cells show higher expression of nephrin, synaptodin, Pax2, podocin, and WT1 compared to pluripotent stem cells. A permeability assay is also used to determine the endocytic uptake of FITC-labeled albumin as further evidence of podocyte-like functional characteristics. Fluorescence microscopy shows FITC-albumin uptake by iPS podocytes is time- and temperature-dependent in iPS podocytes differentiated for 10 days. An uptake of FITC-albumin in the cytoplasm of iPS is distinct compared to control iPS podocytes cultured at 4° C. that do not show endocytic incorporation of albumin.

### Differentiation of Stem-Cell Derived Proximal Tubule Cells

Cells are plated and maintained in a pluripotent state using a method such as that described in example 4. To initiate differentiation, cells are cultured in renal epithelial growth medium (REGM) for 20 days. REGM is supplemented with 0.5% fetal bovine serum (FBS), BMP2 (10 ng/ml) and BMP7 (2.5 ng/ml). To determine collecting proximal tubule identity, aquaporin (AQP)1-expressing cells are detected using qPCR or immunocytochemistry.

### Differentiation of Stem-Cell Derived Distal Tubule Cells and Collecting Duct Cells

Cells are plated and maintained in a pluripotent state using a method such as that described in example 4. Aggregates of cells are treated with BMP for the initial 24 hours to induce differentiation, followed by activin and FGF for the next two days. The induced mesodermal cells are further posteriorized and maintained in the immature mesoderm state in the presence of a high concentration of Wnt agonist (CHIR 10 μM) and Bmp. Embryoid bodies are cultured under these conditions for six days. The induced EBs are harvested at day 14 express multiple signature genes for metanephric nephron progenitors. Immunostaining is performed to show cells coexpressing typical nephrogenic transcription factors, including Wt1, Pax2, Sall1, and Six2. The induced EBs are further dissociated, and the induction efficiency is quantified by cytospin analyses. When the EBs are collected at day 11 (posterior intermediate mesoderm phase), more than 80% of the cells are positive for Wt1. Analysis at day 14 (MM stage) shows that 20%-70% of the cells are positive for each representative metanephric nephron progenitor marker, including Wt1, Pax2, Sall1, and Six2. Furthermore, these induced progenitors exhibit robust tubulogenesis and clustered podocyte formation when cocultured with mouse embryonic spinal cords. Immunohistochemical examinations at day 8 reveal the formation of well-specified nephron components. These structures consist of Wt1/nephrin+glomeruli, cadherin6+ proximal tubules and E-cadherin+ distal tubules, all of which appear to be connected in that order, thereby mimicking human embryonic kidney formation.

### Example 12: Correlation of Glucose Consumption with Vascular Endothelialization

### 1.0 Overview

It was hypothesized that kidney graft glucose consumption could be a marker for its extent of endothelialization, which would indicate the ability of the graft to remain patent long term in vivo. To test this hypothesis, decellularized porcine kidney grafts were recellularized with human endothelial cells and cultured in a perfusion bioreactor, where cell metabolism was monitored daily. Grafts were then implanted in an orthotopic pig model and through a combination of histological and angiographic evaluation, vascular patency in implanted grafts was confirmed to last up to 14 days. Therefore, here we report the longest continuous perfusion of a recellularized kidney graft in vivo to date.

The research objectives of this observational study were:

(1) to test our prespecified hypothesis that in vitro metabolic consumption of glucose correlates with endothelial cell coverage of vasculature in porcine kidneys and provides a non-invasive method for as a functional assessment,
(2) define a threshold GCR that could be used to predict in vivo graft hemocompatibility, and
(3) assess the long-term performance of human endothelialized kidney grafts in an orthotopic transplantation model.

### 2.0 Methods

Histological staining, immunofluorescence imaging, volumetric blood perfusion rates, and angiographies were used to evaluate endothelialized kidney grafts. All endothelialized grafts that were allowed to reach Peak GCR in this study (n=16) are included to demonstrate glucose consumption kinetics over time. Sample size of n=1 decellularized and n=1 endothelialized (Low GCR) grafts were used in ex vivo blood loops for representation of vascular thrombosis, while n=3 endothelialized grafts were used in 3 different ex vivo blood loops to demonstrate repeatability of patent blood flow at Peak GCR. Grafts were excluded when angiography was not available on the day of ex vivo blood loop testing. Sample size of n=9 grafts were implanted in separate pigs to obtain patent grafts at each follow-up time point. Endpoints included animal death, graft thrombosis, and scheduled explantation at each follow-up time point for histological characterization.

### 2.1 Kidney Selection and Recovery

Porcine kidneys weighing 250-300 grams were obtained from adult (approximately 6 months) male and female Landrace/Yorkshire/Duroc cross-breed pigs purchased from Midwest Research Swine (Glencoe, Minn.). Kidneys were rinsed in saline, bagged, and transported on ice to a separate facility for processing.

### 2.2 Decellularization

Kidney pairs were removed from ice and passed into a class 10,000 clean room. All recovered kidneys were disinfected with a pH-neutral peracetic acid before and after cannulation. The renal artery, renal vein, and ureter on each kidney were cannulated using appropriately sized (Range: 1/16″ to ¼″) polypropylene cannulae and secured with 3-0 nylon sutures. Cannulated kidneys were individually bagged in sterile PBS and refrigerated overnight. Kidneys were then perfusion decellularized for 3 hours using Triton X-100 followed by 4-20 hours of 0.6% SDS. Kidneys were then washed with phosphate buffered saline (PBS) and underwent further disinfection using 1000 ppm peracetic acid prior to packaging. Perfusion pressure was held at a constant value (renal artery: 60 mmHg; renal vein: 40 mmHg; ureter: 20 mmHg) by altering the volumetric flow rate of the peristaltic pump, and perfusion was alternated among the renal artery, vein, and ureter. The decellularized kidney scaffolds were then packaged with PBS and stored at 4° C. for up to 3 months.

### 2.3 Endothelial Culture and Seeding

Decellularized kidneys were mounted in sterile bioreactors and were perfused through the vein at 100 mL/min with a pressure less than 20 mmHg in antibiotic-free endothelial cell media (custom endothelial base media, sodium bicarbonate, fetal bovine serum (FBS), ascorbic acid, hydrocortisone, fibroblast growth factor (FGF), vascular endothelial growth factor (VEGF), epidermal growth factor (EGF), recombinant insulin-like growth factor (R3-IGF), heparin, and acetic acid) for at least 3 days to confirm sterility. The media was replaced with endothelial media containing 1% penicillin/streptomycin prior to seeding. Cells used for seeding the kidneys were primary cryopreserved human umbilical vein endothelial cells (HUVECs), passage 5-8, that were expanded in 5-chamber cell culture chambers using antibiotic-free endothelial media. After passaging, HUVECs were passed through a 70 μm strainer and were resuspended at a concentration of 1 million cells/mL. 100 million cells were statically seeded through the vein and then 50 million cells were injected into the venous perfusion flow field at 50 mL/min. After 24 hours, perfusion was switched from the vein to the artery, the media was changed, and another 150 million HUVEC cells were seeded through the artery using the same protocol. At 24 hours after the first arterial seeding, the media was replaced and the flow was increased to 100 mL/min. Metabolite levels in bioreactor media samples were measured daily using a cell culture analyzer. Media was changed on alternating days and glucose consumption rate (GCR) was monitored. Within 1-6 days after the first arterial seeding, the kidney graft was seeded through the artery a second time, using the previously described arterial seeding protocol. The media was changed 24 hours after the serial arterial seeding and then the frequency and volume of media changes was adjusted as needed to prevent total glucose depletion.

### 2.4 Cell Metabolism

Cell metabolic activity was monitored daily by analyzing a bioreactor media sample. Sample metabolite concentrations were obtained from a metabolite analyzer to monitor metabolism of glucose, glutamine, glutamate, ammonia, and lactate dehydrogenase (LDH). When glucose levels fell below 0.2 g/L or ammonia levels rose above 1 mM, additional media was added to the bioreactor.

### 2.5 Ex Vivo Blood Loops

Catheters were placed into the carotid artery and jugular vein in anesthetized adult Yorkshire Cross pigs receiving intravenous heparin injections to maintain activated clotting time between 175-225 seconds. Tygon tubing and barbed Luer adapters were used to create a loop linking the carotid artery and jugular vein. A Transonic flow meter and 8 mm flow probe and pressure T were integrated in the system downstream from carotid access and upstream from the graft. Baseline flow and graft inflow were recorded and angiographies performed over time.

### 2.6 Chronic Kidney Graft Implantation

Kidney grafts were implanted 2-4 weeks after initial recellularized graft seeding depending on targeted GCR. Recipient animals were 75-95 kg Yorkshire Cross pigs. Under general anesthesia and administered heparin to raise the activated clotting time to 175-225 seconds, the kidney grafts were implanted into pigs according to an approved IACUC protocol (American Preclinical Services, IACUC protocol #ZUE036-IS02). Prior to implantation, the kidney graft was flushed with heparinized saline. Each kidney was spray coated with 2 mL fibrin sealant which was allowed to set for 3 minutes. Following a laparotomy, surgeons performed a splenectomy and then implanted the graft by a nephrectomy and subsequent anastomosis of the revascularized graft to the renal vein and artery or by a side anastomosis to the abdominal aorta and inferior vena cava. Vitals and arterial flow to the kidney were monitored using the Transonic flow meter until the flow stabilized and the graft was then immobilized by securing it to the abdominal wall using either the native peritoneum or surgical mesh and the incision was surgically closed. The implants were then monitored for 3, 7, 10, and 14 days or until graft patency was lost. All animals were received and managed under an Institutional Animal Care and Use Committee (IACUC) protocol (American Preclinical Services) where quarantine and veterinary exams were performed. Daily substance administration included aspirin and ketoprofen for pain management, naxcel and exceed for infection prophylaxis, methylprednisolone, and clopidogrel anticoagulation therapy.

### 2.7 Angiography

To obtain angiography data for patency analysis of the kidney grafts, an iohexol contrast agent was injected via catheter. A sheath was placed in the carotid or femoral artery and a catheter advanced to the location of arterial blood supply of the graft. The contrast agent was injected with subtracted angiography using an angiography instrument and software, and images were recorded until the contrast had completely cleared from the kidney. Following the same procedure, contralateral native kidneys (n=3) were analyzed to compare to the patency of the kidney grafts.

### 2.8 Angiographic Analysis

Patency of the kidney grafts was assessed by angiography. Angiography images were acquired at the frame at which the maximum loading of contrast was retained in the kidney. Using ImageJ, a grayscale histogram of pixels was obtained for the region containing the kidney and the pixels with the same grayscale value as the background surrounding the kidney were subtracted. Percent perfusion was then calculated as the percentage of remaining number of pixels retained within the kidney histogram compared to the original number of pixels prior to background subtraction. Results are presented as percent graft perfusion.

### 2.9 Histology

Following euthanization (intravenous barbiturate to effect), the kidney graft was excised and flushed (if patent) with 300 mL PBS and then 300 mL 10% neutral buffered formalin (NBF, VWR). Regions (1 cm thick) were cut from the upper and lower pole as well as the midline of the kidney and were submerged in 10% NBF for 2 days. These samples were then embedded in paraffin, sectioned at 5 μm thickness, and were stained with hematoxylin and eosin (H&E) and Masson's Trichrome staining procedures. Imaging was performed using a color brightfield microscope system, camera, and software.

### 2.10 Immunofluorescence Staining

Paraffin embedded sections were deparaffinized, rehydrated, and subjected to antigen retrieval in a Decloaking Chamber NxGen. Slides were washed with sodium borohydride in PBS to reduce autofluorescent signal from red blood cells. They were then co-stained with appropriate primary and secondary antibodies and DAPI, then mounted using a glycerol based liquid mountant. Imaging was performed using a fluorescent microscope camera and analysis software. Anti-CD31 antibody is reactive to both human and pig endothelial cells. Anti-CD31 antibody is reactive to only human endothelial cells. A co-stain with these antibodies was used to differentiate between HUVEC and recipient pig endothelial cells. Anti-VE-cadherin antibody was used also used to identify human or pig endothelial cells. Anti-human nucleoli antibody was used to identify human endothelial cells. (Anti-Collagen I antibody, Anti-Collagen IV antibody, Anti-Laminin antibody, and AlexaFluor 488 and 555 secondary antibodies were used for matrix characterization.

### 2.11 Statistics

A Kruskal-Wallis ANOVA was performed on the angiography data using a statistical analysis software, where p<0.05 was considered significant. Data are reported as mean standard deviation within text.

### 3.0 Results

### 3.1 Porcine Kidney Decellularization Produces Human-Scale Matrices for Kidney Tissue Regeneration.

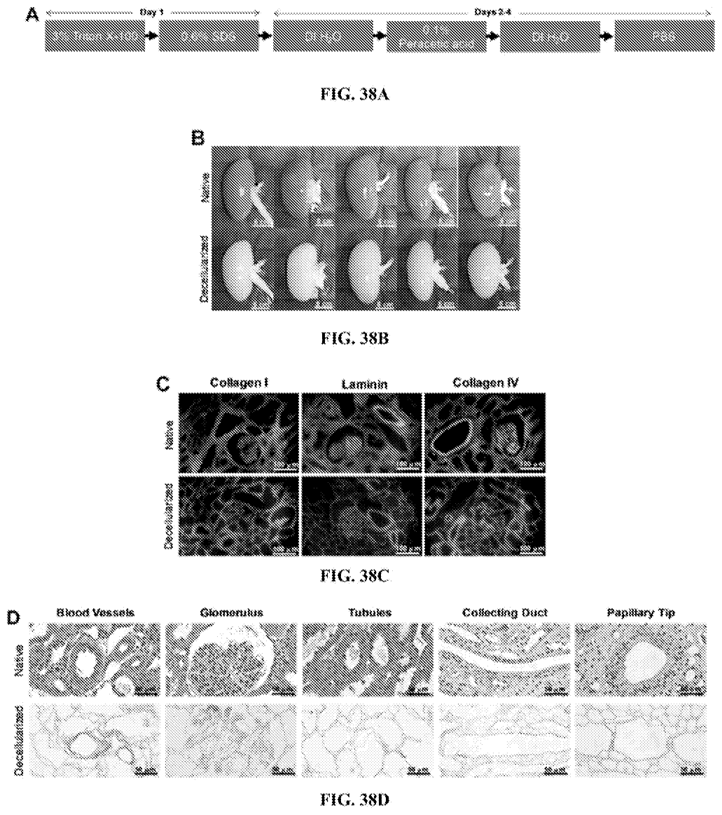

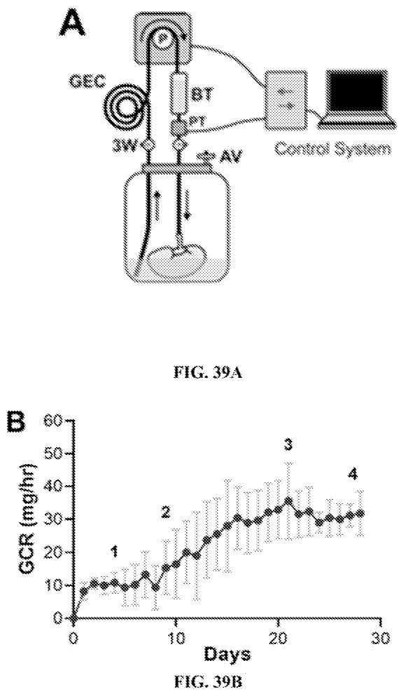

Kidneys were recovered from adult pigs and decellularized via perfusion with a series of detergent solutions sequentially perfused through the vasculature and ureter while perfusing at a specified pressure (FIG. 38A). Decellularized kidneys obtained from adult donor pigs of varying weight largely retained their native gross shape, size, and ECM proteins, including collagen I, laminin, and collagen IV (FIG. 38B and FIG. 38C). The resulting matrix retained the appropriate 3D architecture for renal microstructures, including blood vessels, glomeruli, nephron tubules, collecting ducts, and papillae (FIG. 38D).

### 3.2 Decellularized Porcine Kidney Matrices Support Functional Revascularization by Endothelial Cells

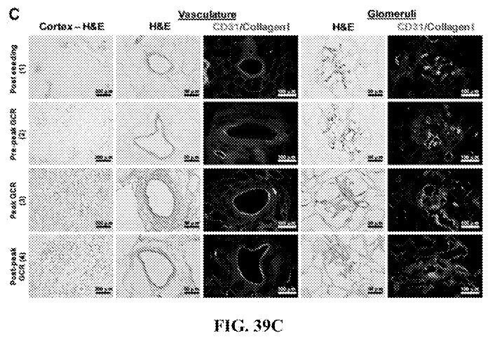

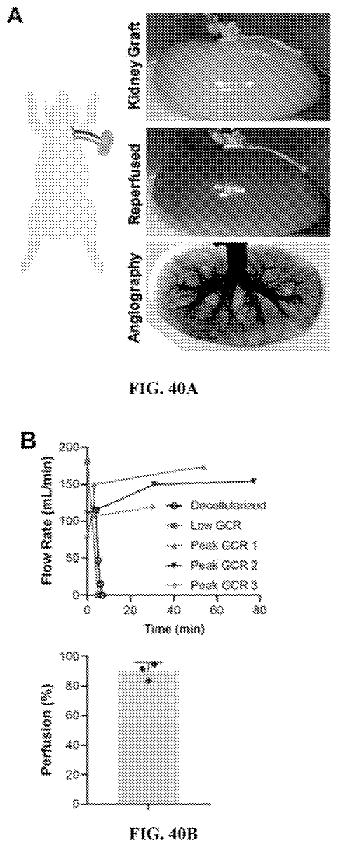

To imbue the porcine matrix with thromboresistance and thereby pave the way for transplantation, decellularized kidney grafts were recellularized by infusing human umbilical vein endothelial cell (HUVEC) suspensions sequentially through the renal vein and artery using a perfusion bioreactor system (FIG. 39A). Custom perfusion software was used to drive the flow of culture media into the kidney through either the renal vein or artery, while maintaining the volumetric flow rate or pressure at a specified value. The engraftment and proliferation of the HUVECs was supported by perfusion with media from a reservoir kept within a humidified incubator, while the pump tubing segment integrated in the perfusion circuit was connected to a peristaltic pump located adjacent to the incubator. This system ensures consistent recellularization among different donor kidneys and across different recellularization lots by closely regulating environmental parameters (e.g., perfusion pressure/flow, temperature, pH, and oxygen tension) during seeding and subsequent perfusion culture.

After HUVEC seeding, cell metabolism was monitored daily by analyzing metabolite concentrations in bioreactor media samples using a bioanalyzer. Total glucose consumption rate (GCR) by endothelialized grafts was monitored over time to gauge the extent of re-endothelialization (FIG. 39B). Early after seeding (0-7 days), the GCR remained low, though HUVECs were found to engraft in large vessels and glomerular capillaries alike (FIG. 2C, “Post Seeding (1)”). After 7-14 days of perfusion culture, the GCR gradually increased, and the density of endothelial cells in blood vessels and glomeruli increased (FIG. 2C, “Pre-peak GCR (2)”). After an average of 21±5 days, GCR reached a peak (41.3±10.2 mg/hr; n=16 grafts), and the native porcine vasculature was reconstituted with HUVECs (FIG. 2C, “Peak GCR (3)”). In this peak period, the endothelial cells were evident in most large blood vessels and glomerular capillaries. After continued culture, GCR would plateau and eventually start to decline following confluency (FIG. 39C, “Post-peak GCR (4)”).

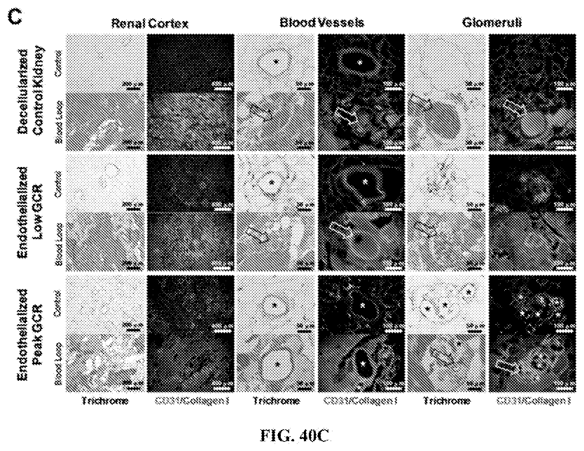

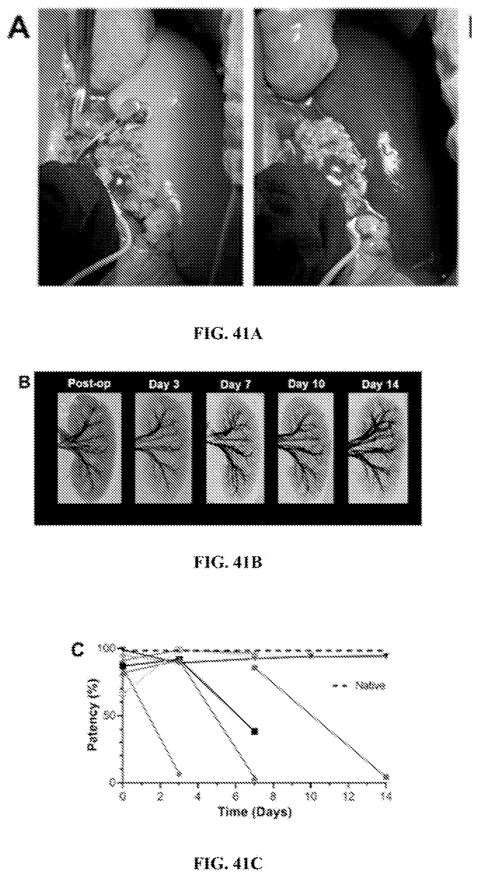

Functional revascularization of decellularized porcine kidneys was tested using an ex vivo blood loop system. Vascular access to anesthetized adult pigs was obtained using catheters, which enabled perfusion of endothelialized kidneys with physiological hemodynamic conditions in an observable setting. This system was used for evaluation of vascular integrity, consistency of tissue perfusion, and distribution of flow across the renal vasculature (FIG. 40A). Decellularized control kidneys thrombosed rapidly within 5-10 minutes, and the inlet volumetric flow rate rapidly declined to zero (FIG. 40B-C). Endothelialized kidneys that produced low GCRs (i.e., below 20 mg/hr) associated with incomplete vascular coverage (FIG. 39C, FIG. 40B, and FIG. 40C) performed similarly. Endothelialized kidneys expressing Peak GCRs (e.g., over 20 mg/hr) could sustain consistent perfusion rates over 100 mL/min after 30 minutes of continuous blood perfusion (78.8±8.6% of baseline values; n=5 grafts). Quantification via angiography stills after a minimum of xx minutes confirmed consistently high percent perfusion of endothelialized grafts (FIG. 40B). Histologically, the vasculature in grafts at Peak GCR remained patent and free of thrombus throughout the recellularized kidney after perfusion with blood (FIG. 40C). Based on these results it was determined that a minimum glucose consumption rate threshold of 20 mg/hr was required for endothelialized grafts to maintain consistent perfusion in ex vivo blood loop.

Re-endothelialized kidney grafts maintain vascular patency for at least 7 days in vivo

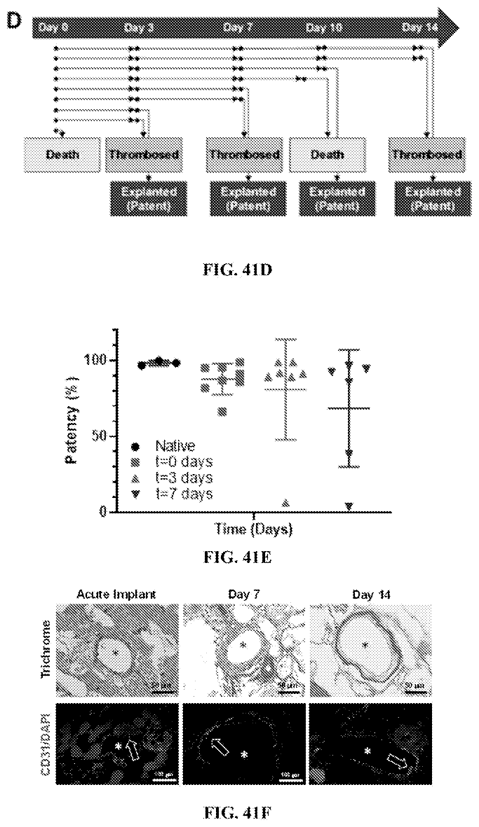

Following confirmation of functional patency in ex vivo blood loops, functional revascularization was evaluated in a chronic, large animal, orthotopic kidney transplantation model. A nephrectomy was performed to excise one native kidney and an endothelialized kidney graft was implanted orthotopically (FIG. 41A). Anastomosis was performed in an end-to-end fashion to the remnant native renal artery and vein, or in an end-to-side fashion to the abdominal aorta and inferior vena cava. Reperfusion was uniform throughout the kidney grafts, with occasional bleeding observed at either the vascular anastomoses or the kidney surface that was halted by use of fibrin sealants as needed. After appropriate positioning of the graft to maintain consistent arterial inflow (measured using a Transonic flow meter), the kidney graft was secured in place using the native peritoneum. Pigs were administered methylprednisolone to mitigate immunological rejection of HUVECs. After observing vascular thrombosis in 3 implanted grafts (at days 3, 7, and 14) around the anastomosis site, daily clopidrogel anticoagulation therapy was administered starting on post-operative day 1.

Patency in 9 different chronically implanted kidney grafts was evaluated using angiography immediately post-reperfusion and at days 3, 7, 10, and 14. Kidney grafts explanted at each follow-up time point showed consistent vascular patency. Grafts perfused uniformly, with contrast reaching all the way out to the cortical edges of the graft (FIG. 41B). The average perfusion percentages for the kidney grafts trended to decrease slightly over time, with native, post-reperfusion, day 3, and day 7 kidneys showing percentages of 98.4±1.6%, 87.7±10.3%, 80.9±33.1%, and 68.5±38.6%, respectively, but the differences were not statistically significant. Furthermore, statistics and standard deviations could not be performed on days 10 and 14 data due to sample sizes, but their average percent perfusion rates were 94.4% and 49.2%, respectively. Additionally, five out of nine kidney grafts were patent with over 80% graft perfusion observed at the final follow-up angiography before termination (FIG. 41C and FIG. 41D). Two pigs died unexpectedly due to excessive bleeding (peri-operatively and internal bleeding on post-operative day 9), and 4 pigs were terminated due to lack of perfusion of the implanted kidney graft (FIG. 41D). Of these 4 kidney grafts explanted, 3 had thrombosed arteries and/or veins upon visual examination and were suspected to result from movement of the graft and vessel torsion after animal recovery. At 7 days after transplantation, 83.3% (n=5/6 pigs) of grafts in surviving animals were still showing renal perfusion during follow-up angiography (FIG. 41C). One kidney graft remained patent through post-operative day 14 (FIG. 41B, FIG. 41C, and FIG. 41D).

Percent perfusion quantified from angiographies was performed on the day of surgery and through POD 7, see FIG. 41E. Percent patency was either consistent with the previous time point or had declined to near-zero due to thrombosis at the arterial inflow (n=1) or venous outflow (n=1) likely from torsion. Additionally, contralateral native kidneys (n=3) were analyzed for comparison. FIG. 41F shows a Trichrome stain showing small (<100 um) blood vessels that remained patent at explant at regular follow-up time intervals following chronic orthotopic transplantation. CD31 stain shows endothelialized blood vessels with open arrows pointing to positive expression. Asterisks denote the lumens of patent blood vessels.

The studies described above show the disclosure produces rBEK constructs with functional vasculature that can retain long-term in vivo vascular patency, thus providing compositions and methods for generating a fully functional transplantable kidney.

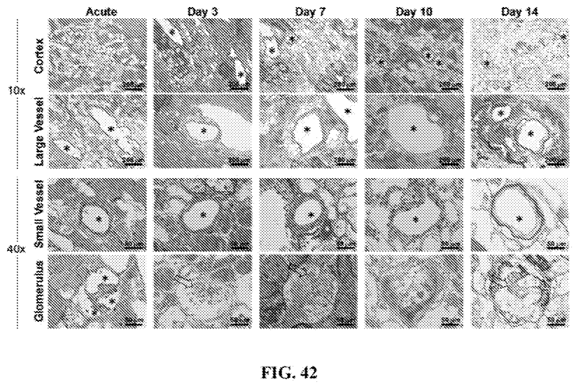

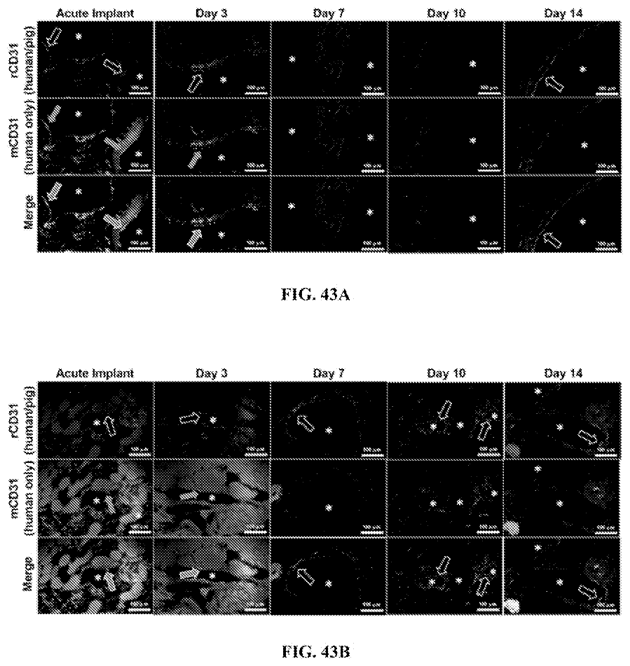

For histological analysis, patent grafts were explanted at regular follow-up endpoints up to 14 days post-transplantation and flushed with saline through the renal artery to remove residual blood before fixation. Following acute transplantation (˜2 hours perfusion), histological trichrome staining showed that the vasculature including glomerular capillaries remained clear of thrombus (FIG. 42). Blood that leaked out of the vasculature into the nephron tubules and surrounding interstitium clotted and filled much of the extravascular matrix. Glomerular capillaries in grafts explanted at subsequent follow-up time points (days 3, 7, 10, and 14) had occluded and were mostly bereft of endothelial cells (FIG. 42). A gradual depletion of human endothelial cells from the graft vasculature was observed and the matrix was completely devoid of the seeded HUVECs by day 7 (FIG. 43A and FIG. 43B). Despite this apparent lack of viable endothelial cells, the vasculature remained patent and the luminal surface did not initiate a clotting cascade, as evidenced by the lack of thrombus (FIG. 42, FIG. 43A, and FIG. 43B). Vascular patency was observed in one implant at day 7 even in the absence of anti-coagulant therapy. By day 7 porcine vascular endothelial cells were observed and localized within the renal vasculature starting with the minor blood vessels (FIG. 43B). By 14 days, many major and minor blood vessel surfaces were re-lined with a continuous monolayer of porcine endothelial cells (FIG. 43A, and FIG. 43B).

### 4.0 Discussion

Perfusion decellularization technology enables the removal of cells and cellular antigens from entire organs which may serve as the renal scaffold for bioengineering of fully functionally human-scale kidneys and thus, potentially eliminate the need for dialysis and the transplant waiting list. However, one design requirement for a transplantable bioengineered organ is a functional vascular supply that can maintain physiological blood flow without thrombosing. Decellularized organs may not meet this requirement due to widespread exposure of the vascular basement membrane to blood, the proteins of which are highly thrombogenic and initiate rapid thrombosis even in an animal treated with heparin (FIG. 40). Therefore, to create a foundation for eventual transplantation of parenchymal-seeded grafts, this study was conducted to first focus on endothelialization of a renal vasculature of decellularized porcine kidneys with human vascular cells.

A renal vasculature branches from a renal artery and vein into interlobar vessels down to individual glomerular capillaries and a vasa recta, which contribute to renal function by facilitating reabsorption/excretion via proximity to an intertwined network of nephron tubules. Endothelialized vessels facilitate patent blood flow in vivo and therefore contribute to survival of implanted bioengineered organs by contributing to the delivery of oxygen and nutrients to parenchymal cells. This approach to re-endothelialization of decellularized porcine scaffolds was developed based on a panel of criteria: 1) adequate histological vascular coverage with endothelial cells, 2) demonstrated increases in cellular metabolism kinetics by non-invasive monitoring, and 3) functional hemocompatibility. All three criteria were used to optimize methods for endothelial cell seeding, recellularized scaffold perfusion culture, and systematic hemodynamic perfusion to ultimately reach predictive outcomes. In some cases, a 10 mg/hr minimum GCR can be adequate for histological endothelial cell coverage and contrast perfusion in recellularized porcine livers. Endothelialized kidney grafts exceeding a minimum GCR threshold of 20 mg/hr in the present study maintained persistent perfusion without thrombosis upon exposure to blood flow. This method allowed for predictability of a readiness of recellularized kidney grafts for transplantation based on non-destructive evaluation.

Differences in human and porcine anatomy can require a surgical approach to kidney transplantation used in this study to differ from a standard retroperitoneal access used clinically. While human patients are typically positioned supine, retroperitoneal access in a pig can require an animal to be positioned on its side, reducing operating space in a surgical field and making vascular anastomosis challenging. In this study, midline laparotomy performed on supine pigs provided a larger surgical field, but allowed positional changes in transplanted grafts after animal recovery that caused the anastomosed vessels to kink or twist, leading to turbulent hemodynamics near the anastomoses. These complications resulted in thrombosis in the renal artery and/or vein, a major source of graft failure in this study. Thrombotic occlusion was mitigated by securing the kidney graft to the abdominal wall using the native peritoneum that stabilized the graft after recovery, thereby prolonging graft patency. In addition, clopidogrel was added to the protocol (starting on post-operative day 1) to specifically reduce the thrombosis observed at the anastomosis sites after an initial 3 kidneys demonstrated continuous perfusion in the absence of anti-coagulation therapy.

Transplanted porcine kidney grafts endothelialized with HUVECs maintained vascular patency for periods lasting up to 2 weeks of post-operative in vivo orthotopic implantation. Angiographies performed at regular follow-up intervals showed that patent grafts maintained consistent and uniform perfusion based on gross qualitative visualization (FIG. 42). Despite administration of steroid immunosuppression therapy, a gradual reduction in HUVEC coverage was observed over time. Considering the excellent performance of the grafts based on angiography (assessed both qualitatively in real-time and quantitatively post-mortem), it was unexpected that most blood vessels had lost human endothelial cell coverage after 7 days remained patent. Vascular thromboresistance initially conferred by endothelial cells seeded in the grafts was likely promoted by clopidogrel anti-coagulation therapy during the observed turnover in endothelial cells. However, 3 initial kidney grafts implanted in animals that did not receive clopidogrel were patent until days 3, 7, and 14, indicating that endothelialized kidneys were capable of sustaining renal blood flow for some time even after endothelial cell depletion from the grafts (between days 3-7) and before re-endothelialization by the porcine host (days 7-14), which suggests that the HUVECs remodeled the ECM of the vasculature resulting in enhanced thromboresistance. While the repopulation of the porcine vasculature by native porcine endothelial cells is encouraging.

In some cases, human cells can be sourced from donor kidneys that have been discarded from a transplant list, such that immunosuppression will still be required, but the availability of kidneys for transplantation can be increased above current levels. In some cases, the methods described herein can be used to circumvent the need for patient immunosuppression by obtaining autologous cells from the intended transplant recipient using biopsy and/or cellular reprogramming via induced pluripotency/directed differentiation.

In summary, this study demonstrates that human-scale bioengineered kidney grafts developed via stepwise perfusion decellularization and re-endothelialization maintained patency with consistent blood flow for up to 14 days in vivo. These encouraging results lay the early foundation for future chronic transplantation of fully functional kidney grafts based upon perfusion decellularized porcine kidneys containing human nephrons.

### Example 13: Biologically Engineered Organs and Portions Thereof

### Bioengineered Liver (BEL) and Portions Thereof

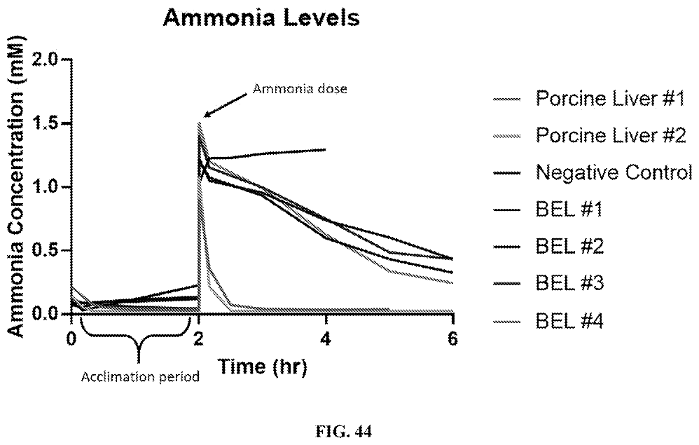

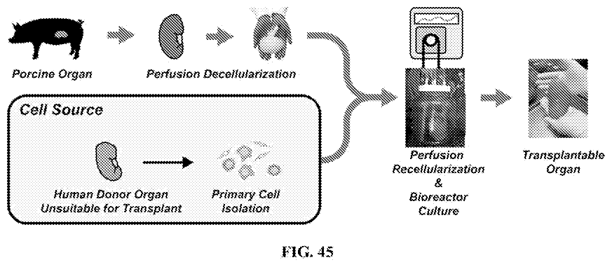

4 revascularized biologically engineered livers (BELs) were seeded with 10 billion hepatocytes (other cells may also be seeded, including but not limited to endothelial cells, cholangiocytes, and/or stem cells capable of differentiating). After the BELs matured, they were installed into a whole blood perfusion loop for 6 hours. The blood was oxygenated for the duration of the perfusion. After a 2 hour acclimation period, the BELs were dosed with additional (excess) ammonia via the circulating blood as a challenge to determine ammonia clearance by the BELs, FIG. 44. Data shows clearance of ammonia by the BELs and supports use of BELs as a therapeutic for subjects experiencing reduced liver function and/or liver failure.

BELs of the disclosure can be utilized to detoxify a subject experiencing acute liver failure. The BEL can be utilized as either a bridge to organ transplant or for maintenance therapy, allowing the subject to recover from increased ammonia, decrease Intrahepatic Cholestasis of Pregnancy (ICP), thereby providing liver function acutely.

### Bioengineered Kidney (BEK) and Portions Thereof

Perfusion decellularization can result in a pristine kidney ECM substantially devoid of cells that retains the original architecture, mechanical properties, and/or biochemical composition of the kidney. Therefore, provided BEK can retain an intact arterial and venous supply to maintain physiological pressures and it can ultimately be surgically anastomosed during implantation.

### Increase Renal Filtration Function Through Glomerular Recellularization

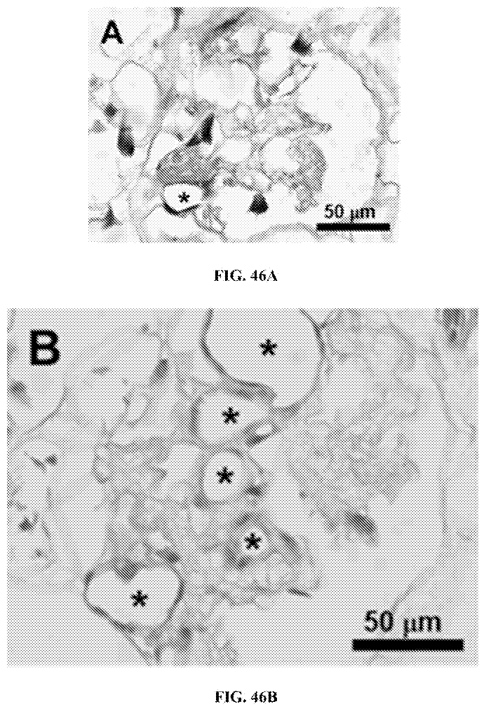

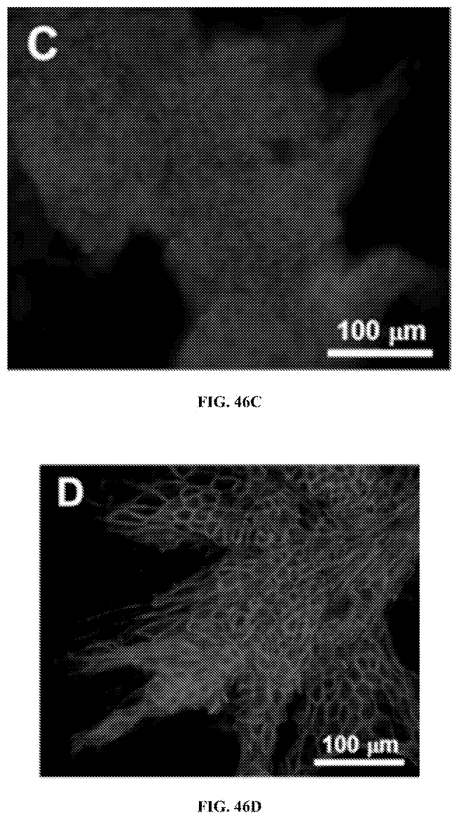

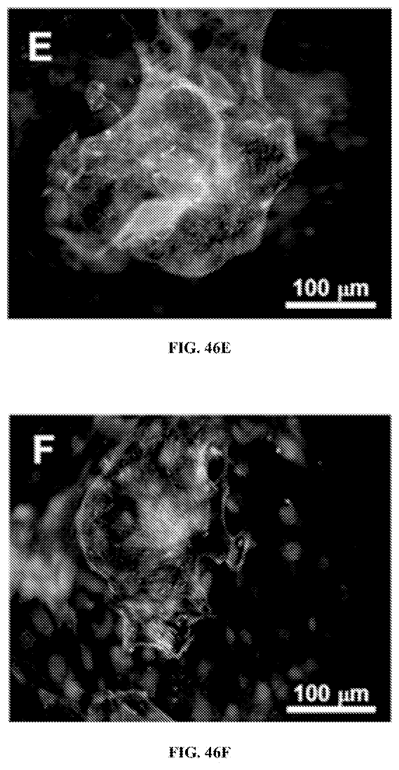

The glomerulus is the primary site of blood filtration whereby the glomerular filtration barrier (GFB) prevents blood cells and large proteins from being excreted while permitting the passage of low molecular weight solutes and water. Forming this filtration barrier are three cell types: endothelial cells, podocytes, and mesangial cells. Methods provided herein show repopulation of the glomerulus with HUVECs with long-term in vivo perfusion. Also provided is the restoration of stable glomerular capillaries with the combination of HUVECs and a mixture of porcine podocytes and mesangial cells after 7 days in vitro (FIG. 46A-FIG. 46B). To isolate glomerular cells, human donor kidneys were perfused with Liberase for 15 minutes and the digested tissue was passed through a series of sieves to purify glomeruli based on size, with an average yield of ˜317,000—+118,000 glomeruli per kidney at 75% purity. Glomeruli were plated on culture dishes for up to 7 days to induce outgrowth of podocytes and mesangial cells, both of which are important for the maintenance of glomerular capillaries. Successful isolation and 2D induction of human podocytes from discarded kidneys is shown in (FIG. 46E-FIG. 46F).

Provided studies show that rBEKs seeded with HUVECs and porcine glomerular cells have albumin retention and creatine clearance capacity about 70% that of native porcine kidneys in bench testing with a plasma analogue (FIG. 46G and FIG. 46H), and effluent flow rate comparable to a native or transplanted porcine kidney in an ex vivo blood loop (FIG. 14C-FIG. 14D). As such, provided is restoration of filtration function in a surrogate BEK. Also provided are glomerulus and epithelial cell isolations using the same processes described above with cell viabilities compared with pig kidneys (Table 2).

TABLE 2

| Kidney Cell Isolation Quality | Porcine | Human |
|---|---|---|
| Glomerulus Purity | 61.5% | 74.6% |
| Glomerulus Yield (×103 glomeruli) | 490 | 317 |
| Glomerulus Plating Efficiency | 99.7% | 83.6% |
| Kidney Digest Cell Viability | 83.7% | 85.6% |
| Kidney Digest Cell Yield (×109 cells) | 11.9 | 1.5 |
| Kidney Digest Cell Plating Efficiency | 38.3% | 76.0% |

Increase BEK Glomerular Recellularization with Endothelial Cells, Mesangial Cells, and Podocytes Utilizing Induction Scheme

Building on the provided endothelialization methods comprising a three-day HUVEC seeding followed by a glomerular seeding, methods defining optimal culture conditions to increase the number of human podocytes seeded while maintaining HUVEC glomerular coverage as measured through immunohistological analysis will be clarified. Perfusion bioreactors will be used for each condition outlined in Table 3 to determine optimal podocyte conditions for histological expression of key podocyte markers including cytoskeletal synaptopodin and filtration slit markers podocin and nephrin.

TABLE 3
Experimental Design

| Podocyte Induction | Induction | Induction | | |
|---|---|---|---|---|
| (Initial expression) | in culture | in Kidney | Day 7 | Day 14 |
| Induction in culture | + | − | n = 4 | n = 4 |
| Induction in kidney | − | + | n = 4 | n = 4 |
| No induction control | − | − | n = 4 | n = 4 |
| Induction in both | + | + | n = 4 | n = 4 |

Glomerular cells will be isolated from human kidneys using the protocol described above. To promote functional expression of primary podocytes in glomerular cultures, a method utilizing low serum concentrations (2% FBS) supplemented with heparin and retinoic acid (RA) will be utilized to reduce mesangial cell growth while podocytes retain their phenotype. After 7 days in induction media, many human glomerular outgrowth cells expressed an arborized podocyte morphology with increased surface area compared to glomerular cells cultured in endothelial media, which contained few podocytes (FIG. 46C-FIG. 46F). HUVEC seeding will be performed using our previously described protocol capable of creating revascularized kidneys, where briefly, 150 million HUVECs will be perfused through the vein (day 0) and through the artery (days 1 and 2). Then on day 3, 100 million podocytes cells will be perfused retrograde through the ureter to engraft them on the periphery of the glomerular capillaries as shown (FIG. 14B, FIG. 46B). To define the optimal seeding conditions for podocyte engraftment and expression of key functional markers, various conditions will be tested. Glomerular isolations will start with a 7-day initial plating in +/− induction medium for cellular outgrowth prior to BEK seeding. Following initial seeding the BEKs will be cultured in +/− induction media to define if pre-induction or post-induction is superior. BEKs (n=4) will be fixed for histological evaluation at each time point (days 7 and 14) for each experimental group (32 total BEKs will be evaluated). Each BEK will be cut into 4 quadrants and 3 random hematoxylin and eosin (H&E)-stained sections of the renal cortex from each will be analyzed, where all glomeruli within the field of view with at least 50% cell coverage will be counted and reported as a percentage of total glomeruli. In addition, the number of capillaries will be counted for all glomeruli in the field of view. IF staining will be used to identify HUVECs (CD31+), podocytes (podocin+, synaptopodin+), and mesangial cells (CD31−/podocin− vimentin+). The percent of recellularized glomeruli with all three cell types will be quantified. Success can be defined by achieving greater than about 5%-30% glomerular recellularization with podocin and synatopodin positive cells. Samples will be processed for electron microscopy to evaluate development of foot processes and fenestrations. To correlate the extent of glomerulus recellularization with measured filtration function, the top 3 conditions will be further studied.

### Increase Renal Filtration Function in Glomerulus-Recellularized BEKs

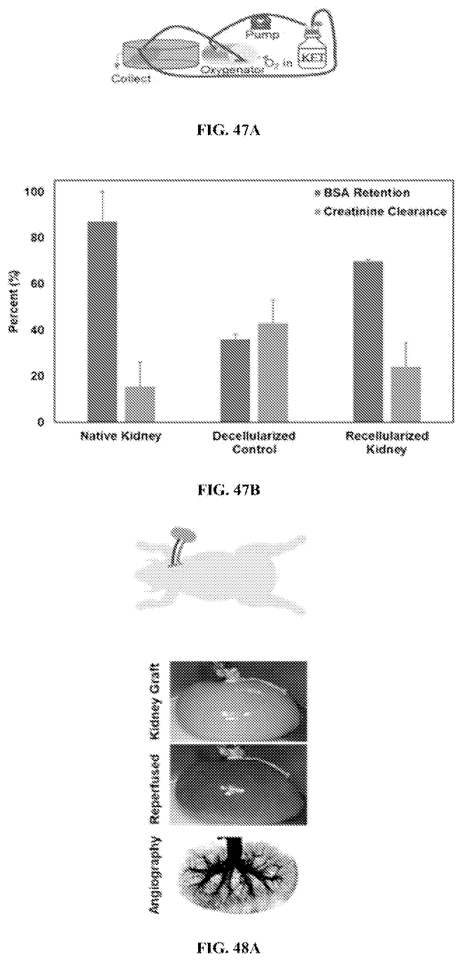

BEKs recellularized using the 3 top conditions from above will be analyzed for filtration function according to our established kidney functional testing assay as provided above (n=4 per condition; n=12 total at days 7 and 14). After perfusing KFT through glomerulus-recellularized BEKs at physiological conditions for 30 min, ureteral effluent volume will be analyzed to calculate albumin retention and creatinine clearance. These rates will be compared to healthy native (n=4) and decellularized (n=4) kidneys (FIG. 47B), where successful BEKs will demonstrate >90% of native kidney albumin retention rates.

### Confirm Hemofiltration of Glomerulus-Recellularized BEKs In Vivo

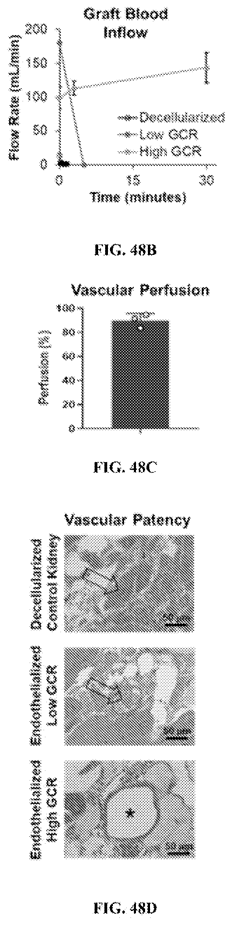

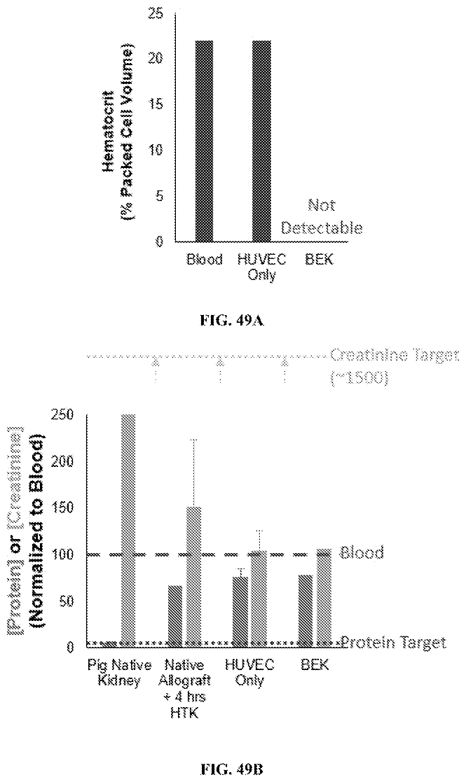

To assess hemofiltration function of BEKs, the top 2 experimental conditions from above (based on measured albumin retention) will be tested in an in vivo kidney perfusion experiment that uses catheter-assisted vascular access in anesthetized pigs. These experiments enable perfusion of kidneys with physiological hemodynamic conditions in an observable setting to evaluate both hemofiltration function and vascular patency based on arterial inflow rate and angiographic imaging (FIG. 48B and FIG. 48C). We have successfully used this in vivo blood loop model to evaluate patency in BEKs (FIG. 48D). The ureteral effluent was collected from the BEKs, and the effluent flow rate for the glomerulus-recellularized BEK was comparable to the native and allogeneic kidney transplant, which was lower than the excessive effluent rate of a rBEK lacking glomerular cells (FIG. 14C-FIG. 14D).

BEKs recellularized using the top two conditions identified will be cultured until a GCR of >20 mg/hr is observed (indicative of complete endothelialization based on preliminary studies) prior to testing. An in vivo blood loop will be created by placing catheters in the carotid artery and jugular vein of an anesthetized pig and connecting them via Luer adapters to the renal artery and vein of the BEKs, respectively (FIG. 48A). Ureter effluent will be analyzed for albumin and creatinine as descried herein. Vascular patency will be determined by Transonic flow measurements of BEK arterial inflow and angiography, and glomerular capillary patency will be evaluated histologically to confirm the ability of human seeded glomeruli to sustain blood flow for a minimum 30 minutes. Allogenic pig kidneys and endothelial-only rBEKs will respectively serve as positive and negative controls for hemofiltration. A total of 16 kidneys will be tested (Top BEK #1, Top BEK #2, pig kidney, rBEK; n=4 for each condition; Table 4). Based on prior experience, 3-4 BEKs will be tested per pig in a total of n=6 pigs (3 male+3 female). To increase rigor, no more than 1 replicate per experimental group will be tested in each pig.

TABLE 4
Experimental Design

| BEK Conditions for In-Vivo | |
|---|---|
| Hemofiltration Function | Day 7 |
| Condition #1 | n = 4 |
| Condition #2 | n = 4 |
| Native Kidney | n = 4 |
| Revascularized BEK | n = 4 |

### Ex-Vivo Porcine Kidney Blood Loop

Functional assessment of recellularized porcine kidneys was tested using an ex vivo blood loop system, which enabled perfusion of the kidneys with physiological hemodynamic conditions in an observable setting. Experimental kidney grafts tested in this experiment were porcine allografts, HUVEC-recellularized kidney grafts, and HUVEC+glomerular outgrowth recellularized kidney grafts. Catheters were placed into the carotid artery and jugular vein in anesthetized adult Yorkshire Cross pigs receiving intravenous heparin injections to maintain activated clotting time between 175-225 seconds. A sample of the pig's blood and urine, prior to exposure to the kidney graft, was taken at this time, where the baseline urine flow rate was also determined. Tygon tubing and barbed Luer adapters were then used to create a loop linking the carotid artery and jugular vein and the kidney graft was hooked into this loop. After 30 minutes of blood perfusion, samples of the pig's blood, urine, and graft effluent were collected, where the graft effluent was timed to determine flow rate of effluent production. All samples were tested for hematocrit, protein, and creatinine content, where experimental samples were normalized to the contents of the pig's circulating blood.

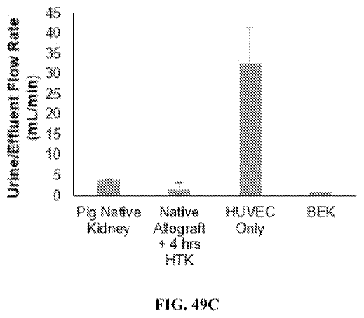

The hematocrit content of the HUVEC-only graft was not significantly different from that of the pig's blood, indicating the HUVEC-only graft was not capable of filtration at the micron level, FIG. 49A. However, the introduction of glomerular outgrowth cells reduced the hematocrit content to an undetectable level. The effluent protein concentrations of the experimental grafts were lower than that of the circulating blood (between 67-79%), indicating that the experimental grafts were able to reduce urine protein loss. As the experimental grafts were not seeded with tubule parenchymal cells, it was not expected to see any increase in creatinine content in the effluent as compared to blood, which is typical of native kidneys. However, the creatinine contents of the experimental grafts were not higher than that of blood, which indicated that the recellularized grafts do not hinder small molecule clearance, see FIG. 49B. Finally, in comparing ureter effluent flow rates, HUVEC-only recellularized grafts had effluent flow rates over 8 times higher than that of native porcine kidneys, see FIG. 49C. Introducing the glomerular outgrowth cells reduced effluent flow rates comparable to that of native porcine kidneys.

What is claimed is:

1. An at least partially recellularized isolated organ or portion thereof comprising at least two different exogenous populations of cells engrafted thereon, wherein the at least partially recellularized isolated organ or portion thereof clears ammonia at a rate of at least 0.1 mmol per hour from a fluid perfused through the vasculature as measured by a flow meter.

2. An at least partially recellularized isolated organ or portion thereof comprising at least two exogenous populations of cells engrafted thereon, wherein the at least partially recellularized isolated organ or portion thereof comprises an at least partially intact vasculature that has a blood flow patency of at least 120 mL/min at about 15 mmHg as measured by a flow meter.

3. An at least partially recellularized isolated organ or portion thereof comprising at least two exogenous populations of cells engrafted thereon, wherein the at least partially recellularized isolated organ or portion thereof comprises an at least partially intact vasculature comprising a circulating fluid, and wherein the at least partially recellularized isolated organ or portion thereof maintains an ammonia concentration of the circulating fluid at a level of less than about 0.4 mM in a time period of about 24 hours as measured by an ammonia analyzer.

4. The at least partially recellularized isolated organ or portion thereof of any one of claim 1, or 3, wherein the fluid comprises blood.

5. The at least partially recellularized isolated organ or portion thereof of any one of claims 1-4, wherein the at least partially recellularized isolated organ or portion thereof is connected to a pump.

6. The at least partially recellularized isolated organ or portion thereof of any one of claims 1-5, wherein the at least two exogenous populations of cells engrafted thereon are allogeneic to the extracellular matrix of the at least partially recellularized isolated organ or portion thereof.

7. The at least partially recellularized isolated organ or portion thereof of any one of claims 1-5, wherein the at least two exogenous populations of cells engrafted thereon are autologous to the extracellular matrix of the at least partially recellularized isolated organ or portion thereof.

8. The at least partially recellularized isolated organ or portion thereof of any one of claims 1-5, wherein the at least two exogenous populations of cells engrafted thereon are xenogeneic to the extracellular matrix of the at least partially recellularized isolated organ or portion thereof.

9. An at least partially recellularized isolated organ or portion thereof comprising a population of engrafted exogenous cells, wherein a density of the population of the exogenous cells in a distal portion of the at least partially recellularized isolated organ or portion thereof comprises at most a 100% difference as compared to a density of the population of the exogenous cells in a proximal portion of the at least partially recellularized isolated organ or portion thereof, as measured by hematoxylin and eosin (H&E) staining of the population of the exogenous cells in the distal portion and the proximal portion of the at least partially recellularized isolated organ or portion thereof.

10. The isolated organ or portion thereof of any one of claims 1-9, further comprising a perfusion solution.

11. The method of claim 10, wherein the perfusion solution comprises at least 120 pO2 mmHg as measured by a Jenway® Model 970 dissolved oxygen meter and electrode.

12. The isolated organ or portion thereof of claim 10, wherein the perfusion solution comprises a growth factor, an immune modulating agent, a coagulation modulating agent, an antibiotic, a preservative, or any combination thereof.

13. The isolated organ or portion thereof of claim 12, wherein the perfusion solution comprises the growth factor, and wherein the growth factor is selected from the group consisting of: Vascular Endothelial Growth Factor (VEGF), Dickkopf WNT Signaling Pathway Inhibitor 1 (DKK-1), Fibroblast Growth Factor (FGF), Bone Morphogenic Protein 1 (BMP-1), Bone Morphogenic Protein 2 (BMP-2), Bone Morphogenic Protein 3 (BMP-3), Bone Morphogenic Protein 4 (BMP-4), Stromal Cell-Derived Factor 1 (SDF-1), Insulin like Growth Factor (IGF), Hepatocyte Growth Factor (HGF), and any combination thereof.

14. The isolated organ or portion thereof of claim 12, wherein the perfusion solution comprises the immune modulating agent, and wherein the immune modulating agent is a cytokine, a glucocorticoid, an interleukin-2 receptor (IL2R) antagonist, a leukotriene antagonist, or any combination thereof.

15. The isolated organ or portion thereof of any one of claims 1-14, wherein the first population of the exogenous cells comprises embryonic stem cells, induced pluripotent stem cells (iPSCs), umbilical cord blood cells, hepatocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), or any combination thereof.

16. The isolated organ or portion thereof of any one of claims 1-15, wherein the at least partially recellularized isolated organ or portion thereof comprises an at least partially recellularized liver or portion thereof, an at least partially recellularized kidney or portion thereof, an at least partially recellularized heart or portion thereof, an at least partially recellularized lung or portion thereof, an at least partially recellularized bowel or portion thereof, an at least partially recellularized skeletal muscle or portion thereof, an at least partially recellularized bone or portion thereof, an at least partially recellularized uterus or portion thereof, an at least partially recellularized bladder or portion thereof, an at least partially recellularized spleen or portion thereof, an at least partially recellularized brain or portion thereof, or an at least partially recellularized pancreas or portion thereof.

17. A system comprising the at least partially recellularized isolated organ or portion thereof of any one of claims 1-16 operatively coupled to a pump.

18. The system of claim 17, wherein the pump is a peristaltic pump or a vacuum pump.

19. The system of claim 17 or 18, wherein the system further comprises a cannula, a perfusion apparatus, a holding container, a tubing, a sensor, a thermometer, an electrode, a valve, a balloon, a pacemaker, a thermostat, a user interface, or any combination thereof.

20. The system of claim 19, wherein the system comprises the sensor, and wherein the sensor comprises a glucose sensor, an ammonia sensor, an oxygen sensor, a fluid sensor, a temperature sensor, a pressure sensor, or any combination thereof.

21. A method comprising introducing a second exogenous population of cells into an at least partially recellularized isolated organ or portion thereof comprising a first exogenous population of engrafted cells, wherein, prior to the introduction of the second population of exogenous cells, at least a portion of the first exogenous population of engrafted cells is functional as determined by:
a. glucose consumption at a rate of at least about 10 mg/h;
b. lactate production at a rate of at least about 30 mg/h;
c. ammonia production at a rate of at least about 0.01 mmol/h;
d. von Willebrand Factor production at a rate of at least about 0.1 ug/h; and
f. any combination thereof.

22. The method of claim 21, wherein the at least partially recellularized isolated organ or portion thereof comprises an at least partially intact vasculature comprising a circulating fluid.

23. The method of claim 22, wherein the circulating fluid comprises blood or a fraction thereof.

24. The method of claim 22, wherein the circulating fluid comprises:
i. a concentration of glucose of from about 0.5 g/L to about 4 g/L,
ii. a concentration of oxygen of from about 120 mmHg to about 400 mmHg,
iii. or any combination thereof.

25. The method of any one of claims 21-24, wherein at least 100% more blood perfusion rate is observed as measured by an external blood loop, compared to an otherwise comparable isolated organ or portion thereof generated by engrafting the second exogenous population of cells onto a decellularized organ or portion thereof that lacks the functional subset of the first exogenous population of cells.

26. The method of any one of claims 21-25, wherein the first exogenous population of engrafted cells comprises endothelial cells.

27. The method of claim 26, wherein the endothelial cells comprise human vein endothelial cells (HUVECs).

28. The method of any one of claims 21-27, wherein the second exogenous population of cells comprises embryonic stem cells, induced pluripotent stem cells (iPSCs), umbilical cord blood cells, hepatocytes, cholangiocytes, podocytes, mesangial cells, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), iPSC derived endothelial cells, differentiated stem cells or any combination thereof.

29. The method of any one of claims 21-28, wherein at least a portion of the second exogenous population of cells comprises liver specific cells or kidney specific cells, wherein the exogenous population of cells are liver specific cells and are hepatocytes or cholangiocytes, and wherein the exogenous population of cells are kidney specific cells and are podocytes or mesangial cells.

30. The method of any one of claims 21-29, wherein the introducing is via a cannula.

31. The method of any one of claims 21-30, wherein at least 25% more of any one of glucose consumption, lactate consumption, oxygen consumption, ribose consumption, and glycogen production is observed in the at least partially recellularized isolated organ or portion thereof as compared to a comparable isolated organ or portion thereof generated by a comparable method absent the functional subset of the first exogenous population of engrafted cells before the introduction of the second exogenous population of cells.

32. The method of any one of claims 21-31, wherein the portion of the first exogenous population of engrafted cells comprises at least 5% of the first exogenous population of engrafted cells.

33. The method of any one of claims 21-32, wherein the at least partially recellularized isolated organ or portion thereof is an at least partially recellularized liver or portion thereof, and wherein the second exogenous population of cells are perfused into the liver or portion thereof via a hepatic vein.

34. The method of any one of claims 21-32, wherein the at least partially recellularized isolated organ or portion thereof is an at least partially recellularized liver or portion thereof, and wherein the second exogenous population of cells are perfused into the liver or portion thereof via a bile duct.

35. The method of any one of claims 21-34, wherein the second exogenous population of cells comprises hepatocytes.

36. The method of any one of claims 21-35, wherein at least one of the populations of exogenous cells are introduced by perfusing a recellularization solution into the at least partially recellularized isolated organ or portion thereof while the at least partially recellularized isolated organ or portion thereof is at least partially submerged in a liquid that comprises the recellularization solution.

37. The method of claim 36, wherein the perfusing is via a cannula.

38. The method of claim 37, wherein the perfusing is antegrade.

39. The method of claim 37, wherein the perfusing is retrograde.

40. A method comprising:
a) determining a concentration of a factor circulating in an at least partially recellularized isolated organ or portion thereof comprising a first population of cells engrafted thereon; and
b) introducing into the at least partially recellularized isolated organ or portion thereof a second population of cells, wherein the first population of cells and the second population of cells are different, and wherein at least one of the first population of cells or the second population of cells are exogenous to the at least partially recellularized isolated organ or portion thereof.

41. The method of claim 40, wherein the factor is glucose, lactate, ammonia, oxygen, ribose, or glycogen.

42. The method of claim 40 or 41, wherein the first population of cells comprises endothelial cells.

43. The method of any one of claims 40-42, wherein the second population of cells comprises embryonic stem cells, induced pluripotent stem cells (iPSCs), umbilical cord blood cells, hepatocytes, cholangiocytes, tissue-derived stem or progenitor cells, dissociated organoids, bone marrow-derived stem or progenitor cells, blood-derived stem or progenitor cells, mesenchymal stem cells (MSC), skeletal muscle-derived cells, multipotent adult progenitor cells (MAPC), cardiac stem cells (CSC), multipotent adult cardiac-derived stem cells, cardiac fibroblasts, cardiac microvasculature endothelial cells, aortic endothelial cells, bone marrow mononuclear cells (BM-MNC), endothelial progenitor cells (EPC), iPSC derived endothelial cells, differentiated stem cells, or any combination thereof.

44. The method of any one of claims 40-43, wherein the at least partially recellularized isolated organ or portion thereof is a liver, a kidney, a heart, a lung, a bowel, a skeletal muscle, a bone, a uterus, a bladder, a spleen, a brain, and a pancreas.

45. The method of any one of claims 40-44, wherein the at least partially recellularized isolated organ or portion thereof is cultured in hyperoxic conditions following the introduction of the second population of cells.

46. The method of claim 45, wherein the hyperoxic conditions comprise an oxygen concentration over 21%, 22%, 23%, or 24% pO₂ 140 mmHg as measured by a Jenway® Model 970 Dissolved Oxygen Meter and Electrode.

47. A method comprising implanting the at least partially recellularized isolated organ or portion thereof of any one of claims 1-16, into a subject.

48. The method of claim 47, wherein the subject has liver disease, hypertension, diabetes, heart failure, lung disease, or kidney disease.

49. The method of claim 48, wherein the subject has the liver disease, and wherein the liver disease is cirrhosis, nonalcoholic steatohepatitis, hepatocellular carcinoma, metabolic disease, or any combination thereof.

50. The method of any one of claims 47-49, further comprising administering an immunosuppressive condition to the subject.

51. The method of any one of claims 47-50, wherein the subject exhibits an intercranial pressure of less than 20 mmHg for a duration of at least two hours during a time period of at least 24 hours after the implanting.

52. A method comprising implanting an isolated at least partially decellularized, at least partially re-endothelialized organ into a subject, wherein said isolated at least partially decellularized, at least partially re-endothelialized organ retains vascular patency for a time period of at least 9 days.

53. The method of claim 52, wherein said at least partially decellularized, at least partially re-endothelialized organ has been at least partially re-endothelialized by contacting with cells that are exogenous to said isolated organ

54. A composition comprising:
(a) an isolated at least partially decellularized, at least partially recellularized organ; and
(b) a circulatory system comprising a liquid, wherein said isolated at least partially decellularized, at least partially recellularized organ substantially retains liquid for at least 9 days.

55. A method comprising revascularizing an isolated at least partially decellularized organ with a cell, the method comprising perfusing said cells through a vein of said organ, then perfusing said cells through an artery of said organ.

56. A system comprising:
(a) an isolated at least partially decellularized, at least partially re-endothelialized organ,
(b) a circulatory system at least partially connected to said isolated at least partially decellularized, at least partially re-endothelialized organ, and
(c) a liquid circulating through said at least partially decellularized, at least partially re-endothelialized organ, and
(d) a sensor configured to measure a component of said media.

57. The method of claim 56, wherein said system is configured to increase a volume of media in said system in response to a measurement of said sensor.

58. The method of claim 56, wherein said component measured by said sensor comprises glucose, glutamine, glutamate, ammonia, lactate dehydrogenase (LDH), or any combination thereof.

59. The method of claim 56, wherein said sensor comprises a glucose sensor.

60. The method of claim 59, wherein said measurement comprises a glucose level of said liquid falling below 0.2 g/L.

61. The method of claim 60, wherein said change of volume comprises an increase of volume in said system.

62. The method of claim 56, wherein said sensor comprises an ammonia sensor.

63. The method of claim 62, wherein said measurement comprises an ammonia level of said liquid increasing above 1 mM.

64. The method of claim 63, wherein said change of volume comprises an increase of volume in said system.

65. A method of at least partially treating kidney failure in a subject in need thereof, comprising grafting an at least partially recellularized kidney onto a circulatory system of the subject, wherein the at least partially recellularized kidney comprises at least a portion of an at least partially intact porcine kidney extracellular matrix comprising xenogeneic or allogeneic glomerular cells engrafted thereon prior to the grafting, wherein the grafting:
(a) reduces a level of hematocrit in the blood of the subject, relative to a level of hematocrit in the blood prior to the grafting,
(b) reduces an effluent protein concentration of the blood of the subject, relative to a protein concentration in the blood prior to the grafting;
(c) is sufficient to produce an effluent flow rate that is comparable to a native porcine kidney; or
(d) any combination thereof, thereby at least partially treating the kidney failure in the subject.

66. The method of claim 65, wherein the glomerular cells comprise podocytes.

67. The method of claim 65, wherein the glomerular cells comprise mesangial cells.

68. The method of any one of claims 65-67, wherein the glomerular cells comprise human cells.
